(19) United States
(12) Patent Application Publication
(10) Pub. No.: US 2026/0146271 A1
(43) Pub. Date: May 28, 2026

(54) UDP-SUGAR BIOPRODUCTION USING MICROORGANISM HOSTS

(71) Applicant: DEBUT BIOTECHNOLOGY, INC., San Diego, CA (US)

(72) Inventors: Nicholas Brideau, San Diego, CA (US); Jingyi Li, San Diego, CA (US); Johannes Van Dijk, San Diego, CA (US)

(21) Appl. No.: 18/996,383

(22) PCT Filed: Jul. 21, 2023
(86) PCT No.: PCT/US2023/070677

§ 371 Date: Jan. 17, 2025

Related U.S. Application Data
(60) Provisional application No. 63/391,395, filed on Jul. 22, 2022.

(51) Int. Cl. C12P 19/38

(52) U.S. Cl. CPC C12P 19/385

(57) ABSTRACT

The present invention is directed to compositions and methods for production of UDP-sugars, including UDP-glucose and UDP-galactose. In particular, the invention provides engineered cell lines for production of UDP-sugars and methods of production of UDP-sugars with these engineered cell lines.

### I. FIELD OF THE INVENTION

The invention is related to materials, including engineered cells and cell lines, and methods involved in the production of uridine diphosphate (UDP) sugars including UDP-glucose and UDP-galactose.

### II. SEQUENCE LISTING

The application contains a sequence listing in electronic format. The Sequence Listing is provided as a file named DEBU-012-01WO-Seq-Listing.xml, created on Jul. 20, 2023, which is 98 KB in size. The information in the electronic format of the sequence listing is incorporated herein by reference in its entirety.

### III. BACKGROUND OF THE INVENTION

Nucleotide sugars are the key precursors for all glycosylation reactions and are required both for oligo- and polysaccharides synthesis and protein and lipid glycosylation. Among all nucleotide sugars, UDP-sugars are the most important precursors for biomass production in nature. UDP-galactose is used for the synthesis of galactolipids, such as monogalactosyldiacylglycerol and digalactosyldiacylglycerol. UDP-galactose is also used as precursor for galactinol, which together with sucrose is used for the synthesis of polysaccharides.

Uridine diphosphate glucose (UDP-glucose), is one of nucleotide sugars widely distributed in cells of microorganisms, animals and plants, and is used as a glucose donor in the biosynthesis of various glycosides, oligosaccharides and polysaccharides. In addition, Uridine-5′-diphosphate-glucose (UDP-glucose) is a fundamentally important molecule in biology, food, biopharmaceuticals and cosmetic chemistry. It is one of the key precursors for sugar interconversion, for formation of di- and polysaccharides, and in amino and nucleotide sugar metabolism. In addition, UDP-glucose can be used as a source for other industrially interesting compounds such as antibiotics.

A number of chemical methods for UDP-glucose synthesis have already been proposed, but they create reactivity and selectivity problems, often requiring modification of functional groups to protect those residues of the sugar molecules that should not react, and at the same time expose those groups that should react. Furthermore, these chemical reactions often require expensive catalysts and organic solvents. As a result, the chemical synthesis of UDP-glucose is not cost-efficient and not environment-friendly.

### IV. SUMMARY OF THE INVENTION

To move away from chemically derived products, the invention provides engineered cells for the bioproduction of UDP-sugars. This approach provides a feasible route for the rapid, safe, economical, and sustainable production of an important set of molecules. The invention provides a feasible route for the rapid, safe, economical, and sustainable production of UDP-sugars including, but not limited to, UDP-glucose and UDP-galactose. Aspects of the invention are accomplished with engineered cells for the bioproduction of UDP-sugars through engineered host cells comprising one or more genetic modifications. Herein, UDP-sugars are biomanufactured using a modified microbial host. Herein, the engineered cells include one or more genetic modifications that increase(s) the bioproduction of UDP-sugars, including UDP-glucose and UDP-galactose.

Provided herein are cells engineered for the production of UDP-glucose, where the engineered cells include one or more genetic modifications that increase UDP-glucose production by increasing metabolic flux to UDP-glucose precursors and/or reducing carbon losses resulting from the production of byproducts. As nonlimiting examples, a genetic modification can be a modification for over-expressing or under-expressing one or more endogenous genes in the engineered host cell or can be a modification for expressing one or more non-native genes in the engineered host cell. Engineered cells as provided herein can include multiple genetic modifications.

Provided herein are cells engineered for the production of UDP-galactose, where the engineered cells include one or more genetic modifications that increase UDP-galactose production by increasing metabolic flux to UDP-galactose precursors and/or reducing carbon losses resulting from the production of byproducts. As nonlimiting examples, a genetic modification can be a modification for over-expressing or under-expressing one or more endogenous genes in the engineered host cell or can be a modification for expressing one or more non-native genes in the engineered host cell. Engineered cells as provided herein can include multiple genetic modifications.

Provided herein are cells engineered for the production of UDP-glucose from sucrose, where the engineered cells include one or more genetic modifications that increase UDP-glucose production by increasing metabolic flux to UDP-glucose precursors and/or reducing carbon losses resulting from the production of byproducts. As nonlimiting examples, a genetic modification can be a modification for over-expressing or under-expressing one or more endogenous genes in the engineered host cell or can be a modification for expressing one or more non-native genes in the engineered host cell. Engineered cells as provided herein can include multiple genetic modifications.

Further provided are methods for producing UDP-sugars that include culturing a cell engineered for the production of UDP-sugars as provided herein. As nonlimiting examples, a genetic modification can be a modification for over-expressing or under-expressing one or more endogenous genes in the engineered host cell or can be a modification for expressing one or more non-native genes in the engineered host cell. Engineered cells as provided herein can include multiple genetic modifications.

In one aspect, the invention provides an engineered host cell that comprises one or more genetic modifications that result in enzymatic transformation by the engineered host cell of a carbon source through multiple chemical intermediates into a UDP-sugar. In certain embodiments, the UDP-sugar is glucose. In certain embodiments, the UDP-sugar is UDP-galactose. In certain embodiments, the chemical intermediate is UDP-glucose and the UDP-sugar is UDP-galactose. In certain embodiments, the engineered host cell is E. coli. In certain embodiments, the carbon source is selected from a group consisting of glycerol, glucose, sucrose, galactose, fructose or any combination thereof.

In certain embodiments, the one or more genetic modifications in the engineered host cell result in an increased production of UDP-glucose. In certain embodiments, the one or more genetic modifications resulted in an increased production of UDP-glucose are overexpression of one or more genes selected from a group consisting of: (i) glucose facilitator gene (glf) or a homolog thereof, (ii) glucokinase (glk) or a homolog thereof, (iii) phosphoglucomutase (pgm) or a homolog thereof, (iv) UTP-glucose-1-phosphate-uridylyltransferase (galU) or a homolog thereof, (v) fructokinase (cscK) or a homolog thereof, and (vi) any combination thereof.

In certain embodiments, the one or more genetic modifications resulted in an increased production of UDP-glucose are downregulation or deletion of one or more genes selected from a group consisting of: (i) glucose-6-phosphate isomerase (pgi); (ii) glucose-1-phosphate adenylyltransferase (glgC); (iii) UDP-glucose 6-dehydrogenase (ugd); (iv) glucans biosynthesis glucosyltransferase (OpgG); (v) UDP-glucose 4-epimerase (galE); (vi) UDP-sugar hydrolase (ushA); (vii) UTP-glucose-1-phosphate uridylyltransferase (ugp), (viii) phosphoglucomutase (pgm), (ix) (heptosyl) lipopolysaccharide α-1,3-glucosyltransferase (WaaG), (x) trehalose-6-phosphate synthase (otsA), (xi) glucose-1-phosphatase (agp), and (xii) any combination thereof. In certain embodiments, the one or more genetic modifications resulted in an increased production of UDP-glucose are downregulation or deletion of phosphoglucomutase (pgm). In certain embodiments, the engineered host cell is supplemented with a medium comprising glucose. In certain embodiments, the engineered host cell is supplemented with a medium comprising fructose.

In certain embodiments, the one or more genetic modifications resulted in an increased production of UDP-glucose is overexpression of one or more genes selected from a group consisting of: (i) sucrose transporter (cscB) or a homolog thereof, (ii) sucrose synthase (SuSy) or a homolog thereof, (iii) sucrose phosphorylase (SPase) or a homolog thereof, and (iv) any combination thereof. In certain embodiments, the engineered host cell is supplemented with a medium comprising sucrose.

In certain embodiments, the one or more genetic modifications in the engineered host cell result in an increased production of UDP-glucose. In certain embodiments, the one or more genetic modifications resulted in an increased production of UDP-glucose are overexpression of one or more genes selected from a group consisting of: (i) glucose-6-phopshate 1-dehydrogenase (zwf) or a homolog thereof; (ii) 6-phosphogluconate dehydrogenase (gnd) or a homolog thereof; (iii) phosphoribosylpyrophosphate synthetase (prs) or a homolog thereof; (iv) phosphoribosyltransferase (pyrE) or a homolog thereof; (v) orotidine 5′-phosphate decarboxylase (pyrF) or a homolog thereof, (vi) uridylate kinase (pyrH) or a homolog thereof, (vii) nucleoside diphosphate kinase (ndk) or a homolog thereof, and (viii) adenylate kinase (adk) or a homolog thereof, and (ix) any combination thereof. In certain embodiments, the engineered host cell is supplemented with a medium comprising orotic acid.

In certain embodiments, the one or more genetic modifications in the engineered host cell result in an increased production of UDP-glucose. In certain embodiments, the one or more genetic modifications in the engineered host cell resulting in an enhanced production of UDP-glucose are selected from: overexpression of one or more genes selected from a group consisting of: glucose facilitator gene (glf) or a homolog thereof, glucokinase (glk) or a homolog thereof, phosphoglucomutase (pgm) or a homolog thereof, UTP-glucose-1-phosphate-uridylyltransferase (galU) or a homolog thereof, sucrose transporter (cscB) or a homolog thereof, sucrose synthase (SuSy) or a homolog thereof, sucrose phosphorylase (SPase) or a homolog thereof, glucose-6-phopshate 1-dehydrogenase (zwf) or a homolog thereof; 6-phosphogluconate dehydrogenase (gnd) or a homolog thereof; phosphoribosylpyrophosphate synthetase (prs) or a homolog thereof; phosphoribosyltransferase (pyrE) or a homolog thereof; orotidine 5′-phosphate decarboxylase (pyrF) or a homolog thereof, uridylate kinase (pyrH) or a homolog thereof, nucleoside diphosphate kinase (ndk) or a homolog thereof, and adenylate kinase (adk) or a homolog thereof, fructokinase (cscK) or a homolog thereof; and/or downregulation or deletion of one or more genes selected from a group consisting of: glucose-6-phosphate isomerase (pgi), glucose-1-phosphate adenylyltransferase (glgC), UDP-glucose 6-dehydrogenase (ugd), glucans biosynthesis glucosyltransferase (OpgG), UDP-glucose 4-epimerase (galE), UDP-sugar hydrolase (ushA), UTP-glucose-1-phosphate uridylyltransferase (ugp), phosphoglucomutase (pgm), (heptosyl) lipopolysaccharide α-1,3-glucosyltransferase (WaaG), trehalose-6-phosphate synthase (otsA), glucose-1-phosphatase (agp); and/or any combinations thereof. In certain embodiments, the engineered host cells are supplemented with a medium comprising: orotic acid, sucrose, glucose, fructose, or a combination thereof.

In certain embodiments, the one or more genetic modifications in the engineered host cell result in an increased production of UDP-galactose. In certain embodiments, the one or more genetic modifications in the engineered host cell resulting in an enhanced production of UDP-galactose are selected from: (i) overexpression of UDP-glucose 4-epimerase (galE) or a homolog thereof, (ii) overexpression of galactokinase (galK) or a homolog thereof, (iii) overexpression of galactose-1-phosphate uridylyltransferase (galT) or a homolog thereof, and (iv) any combinations thereof. In certain embodiments, the engineered host cells are supplemented with a medium comprising galactose.

In certain preferred embodiments, the one or more genetic modifications in the engineered host cell for increasing the production of UDP-galactose include one or more genetic modifications resulting in an increased production of UDP-glucose. Accordingly, in certain preferred embodiments, the one or more genetic modifications to increase the production of UDP-galactose are selected from: overexpression of one or more genes selected from a group consisting of: glucose facilitator gene (glf) or a homolog thereof, glucokinase (glk) or a homolog thereof, phosphoglucomutase (pgm) or a homolog thereof, UTP-glucose-1-phosphate-uridylyltransferase (galU) or a homolog thereof, sucrose transporter (cscB) or a homolog thereof, sucrose synthase (SuSy) or a homolog thereof, sucrose phosphorylase (SPase) or a homolog thereof, glucose-6-phopshate 1-dehydrogenase (zwf) or a homolog thereof; 6-phosphogluconate dehydrogenase (gnd) or a homolog thereof; phosphoribosylpyrophosphate synthetase (prs) or a homolog thereof; phosphoribosyltransferase (pyrE) or a homolog thereof; orotidine 5′-phosphate decarboxylase (pyrF) or a homolog thereof, uridylate kinase (pyrH) or a homolog thereof, nucleoside diphosphate kinase (ndk) or a homolog thereof, and adenylate kinase (adk) or a homolog thereof; overexpression of UDP-glucose 4-epimerase (galE) or a homolog thereof, overexpression of galactokinase (galK) or a homolog thereof, overexpression of galactose-1-phosphate uridylyltransferase (galT) or a homolog thereof; and/or downregulation or deletion of one or more genes selected from a group consisting of: glucose-6-phosphate isomerase (pgi), glucose-1-phosphate adenylyltransferase (glgC), UDP-glucose 6-dehydrogenase (ugd), glucans biosynthesis glucosyltransferase (OpgG), UDP-sugar hydrolase (ushA), UTP-glucose-1-phosphate uridylyltransferase (ugp), phosphoglucomutase (pgm); and/or any combinations thereof. In certain embodiments, the engineered host cells are supplemented with a medium comprising: glucose, galactose, orotic acid, sucrose, or any combination thereof.

In another aspect, the invention provides a method for increasing production of UDP-sugars, the method comprising: providing an engineered host cell that comprises one or more genetic modifications that result in enzymatic transformation by the engineered host cell of a carbon source through multiple chemical intermediates into a UDP-sugar. In certain embodiments, the UDP-sugar is glucose. In certain embodiments, the UDP-sugar is UDP-galactose. In certain embodiments, the chemical intermediate is UDP-glucose and the UDP-sugar is UDP-galactose. In certain embodiments, the engineered host cell is E. coli. In certain embodiments, the carbon source is selected from a group consisting of glycerol, glucose, sucrose, galactose, fructose, or any combinations thereof.

In certain embodiments, the method comprises one or more genetic modifications in the engineered host cell resulting in an increased production of UDP-glucose. In certain embodiments, the one or more genetic modifications resulting in an increased production of UDP-glucose are overexpression of one or more genes selected from a group consisting of: (i) glucose facilitator gene (glf) or a homolog thereof, (ii) glucokinase (glk) or a homolog thereof, (iii) phosphoglucomutase (pgm) or a homolog thereof, (iv) UTP-glucose-1-phosphate-uridylyltransferase (galU) or a homolog thereof, and (v) any combination thereof. In certain embodiments, the one or more genetic modifications resulted in an increased production of UDP-glucose are downregulation or deletion of one or more genes selected from a group consisting of: (i) glucose-6-phosphate isomerase (pgi); (ii) glucose-1-phosphate adenylyltransferase (glgC); (iii) UDP-glucose 6-dehydrogenase (ugd); (iv) glucans biosynthesis glucosyltransferase (OpgG); (v) UDP-glucose 4-epimerase (galE); (vi) UDP-sugar hydrolase (ushA); (vii) UTP-glucose-1-phosphate uridylyltransferase (ugp), (viii) phosphoglucomutase (pgm), (ix) (heptosyl) lipopolysaccharide α-1,3-glucosyltransferase (WaaG), (x) trehalose-6-phosphate synthase (otsA), (xi) glucose-1-phosphatase (agp), and (xii) any combination thereof. In certain embodiments, the one or more genetic modifications resulting in an increased production of UDP-glucose are downregulation or deletion of phosphoglucomutase (pgm). In certain embodiments, the engineered host cell is supplemented with a medium comprising glucose. In certain embodiments, the engineered host cell is supplemented with a medium comprising fructose.

In certain embodiments, the method comprises one or more genetic modifications resulting in an increased production of UDP-glucose are overexpression of one or more genes selected from a group consisting of: (i) sucrose transporter (cscB) or a homolog thereof, (ii) sucrose synthase (SuSy) or a homolog thereof, (iii) sucrose phosphorylase (SPase) or a homolog thereof, and (iv) any combination thereof. In certain embodiments, the engineered host cell is supplemented with a medium comprising sucrose.

In certain embodiments, the method comprises one or more genetic modifications in the engineered host cell resulting in an increased production of UDP-glucose. In certain embodiments, the one or more genetic modifications resulted in an increased production of UDP-glucose are overexpression of one or more genes selected from a group consisting of: (i) glucose-6-phopshate 1-dehydrogenase (zwf) or a homolog thereof; (ii) 6-phosphogluconate dehydrogenase (gnd) or a homolog thereof; (iii) phosphoribosylpyrophosphate synthetase (prs) or a homolog thereof; (iv) phosphoribosyltransferase (pyrE) or a homolog thereof; (v) orotidine 5′-phosphate decarboxylase (pyrF) or a homolog thereof, (vi) uridylate kinase (pyrH) or a homolog thereof, (vii) nucleoside diphosphate kinase (ndk) or a homolog thereof, and (viii) adenylate kinase (adk) or a homolog thereof, and (ix) any combination thereof. In certain embodiments, the engineered host cell is supplemented with a medium comprising orotic acid.

In certain embodiments, the method comprises one or more genetic modifications in the engineered host cell resulting in an increased production of UDP-glucose. In certain embodiments, the one or more genetic modifications in the engineered host cell resulting in an enhanced production of UDP-glucose are selected from: overexpression of one or more genes selected from a group consisting of: glucose facilitator gene (glf) or a homolog thereof, glucokinase (glk) or a homolog thereof, phosphoglucomutase (pgm) or a homolog thereof, UTP-glucose-1-phosphate-uridylyltransferase (galU) or a homolog thereof, sucrose transporter (cscB) or a homolog thereof, sucrose synthase (SuSy) or a homolog thereof, sucrose phosphorylase (SPase) or a homolog thereof, glucose-6-phopshate 1-dehydrogenase (zwf) or a homolog thereof; 6-phosphogluconate dehydrogenase (gnd) or a homolog thereof; phosphoribosylpyrophosphate synthetase (prs) or a homolog thereof; phosphoribosyltransferase (pyrE) or a homolog thereof; orotidine 5′-phosphate decarboxylase (pyrF) or a homolog thereof, uridylate kinase (pyrH) or a homolog thereof, nucleoside diphosphate kinase (ndk) or a homolog thereof, and adenylate kinase (adk) or a homolog thereof, fructokinase (cscK) or a homolog thereof; and/or downregulation or deletion of one or more genes selected from a group consisting of: glucose-6-phosphate isomerase (pgi), glucose-1-phosphate adenylyltransferase (glgC), UDP-glucose 6-dehydrogenase (ugd), glucans biosynthesis glucosyltransferase (OpgG), UDP-glucose 4-epimerase (galE), UDP-sugar hydrolase (ushA), UTP-glucose-1-phosphate uridylyltransferase (ugp), phosphoglucomutase (pgm), (heptosyl) lipopolysaccharide α-1,3-glucosyltransferase (WaaG), trehalose-6-phosphate synthase (otsA), glucose-1-phosphatase (agp); and/or any combinations thereof. In certain embodiments, the engineered host cells are supplemented with a medium comprising: orotic acid, sucrose, glucose, fructose, or a combination thereof.

In certain embodiments, the method comprises one or more genetic modifications in the engineered host cell resulting in an increased production of UDP-galactose. In certain embodiments, the one or more genetic modifications in the engineered host cell resulting in an enhanced production of UDP-galactose are selected from: (i) overexpression of UDP-glucose 4-epimerase (galE) or a homolog thereof, (ii) overexpression of galactokinase (galK) or a homolog thereof, (iii) overexpression of galactose-1-phosphate uridylyltransferase (galT) or a homolog thereof, and (iv) any combinations thereof. In certain embodiments, the engineered host cells are supplemented with a medium comprising galactose.

In certain preferred embodiments, the method provides that one or more genetic modifications in the engineered host cell for increasing the production of UDP-galactose include one or more genetic modifications resulting in an increased production of UDP-glucose. Accordingly, in certain preferred embodiments, the one or more genetic modifications to increase the production of UDP-galactose are selected from: overexpression of one or more genes selected from a group consisting of: glucose facilitator gene (glf) or a homolog thereof, glucokinase (glk) or a homolog thereof, phosphoglucomutase (pgm) or a homolog thereof, UTP-glucose-1-phosphate-uridylyltransferase (galU) or a homolog thereof, sucrose transporter (cscB) or a homolog thereof, sucrose synthase (SuSy) or a homolog thereof, sucrose phosphorylase (SPase) or a homolog thereof, glucose-6-phopshate 1-dehydrogenase (zwf) or a homolog thereof; 6-phosphogluconate dehydrogenase (gnd) or a homolog thereof; phosphoribosylpyrophosphate synthetase (prs) or a homolog thereof; phosphoribosyltransferase (pyrE) or a homolog thereof; orotidine 5′-phosphate decarboxylase (pyrF) or a homolog thereof, uridylate kinase (pyrH) or a homolog thereof, nucleoside diphosphate kinase (ndk) or a homolog thereof, and adenylate kinase (adk) or a homolog thereof; overexpression of UDP-glucose 4-epimerase (galE) or a homolog thereof, overexpression of galactokinase (galK) or a homolog thereof, overexpression of galactose-1-phosphate uridylyltransferase (galT) or a homolog thereof, fructokinase (cscK) or a homolog thereof; and/or downregulation or deletion of one or more genes selected from a group consisting of: glucose-6-phosphate isomerase (pgi), glucose-1-phosphate adenylyltransferase (glgC), UDP-glucose 6-dehydrogenase (ugd), glucans biosynthesis glucosyltransferase (OpgG), UDP-sugar hydrolase (ushA), UTP-glucose-1-phosphate uridylyltransferase (ugp), phosphoglucomutase (pgm), (heptosyl) lipopolysaccharide α-1,3-glucosyltransferase (WaaG), trehalose-6-phosphate synthase (otsA), glucose-1-phosphatase (agp); and/or any combinations thereof. In certain embodiments, the engineered cell is the engineered host cells are supplemented with a medium comprising: glucose, galactose, orotic acid, sucrose, and any combinations thereof.

In another aspect, the invention provides an engineered host cell that comprises one or more genetic modifications that result in enzymatic transformation by the engineered host cell of a carbon source through multiple chemical intermediates to generate UDP-glucose. In certain embodiments, the one or more genetic modifications is overexpression of glucose facilitator gene (glf) or a homolog thereof. In certain embodiments, the one or more genetic modifications is overexpression of glucokinase (glk) or a homolog thereof. In certain embodiments, the one or more genetic modifications are at least one genetic modification selected from the group consisting of: (i) one or more modifications for over-expressing one or more endogenous genes in the engineered host cells; (ii) one or more modifications for under-expressing one or more endogenous genes in the engineered host cells; (iii) one or more genetic modification is expressing one or more non-native genes in the engineered host cells; and (iv) a combination thereof. In certain embodiments, the engineered host cell is E. coli. In certain embodiments, the one or more genetic modifications is overexpression of one or more genes selected from a group consisting of: (i) glucokinase (glk) or a homolog thereof, (ii) glucose facilitator gene (glf) or a homolog thereof, (iii) phosphoglucomutase (pgm) or a homolog thereof, (iv) UTP-glucose-1-phosphate-uridylyltransferase (galU) or a homolog thereof, (v) orotate phosphoribosyltransferase (pyrE) or a homolog thereof, (vi) orotidine 5′-phosphate decarboxylase (pyrF) or a homolog thereof. (vii) uridylate kinase (pyrH) or a homolog thereof, (viii) nucleoside diphosphate kinase (ndk) or a homolog thereof, (ix) adenylate kinase (adk) or a homolog thereof; (x) fructokinase (cscK) or a homolog thereof, and (xi) any combinations thereof. In certain embodiments, the one or more genetic modifications is overexpression of one or more genes selected from a group consisting of: (i) glucose facilitator gene (glf) or a homolog thereof, (ii) glucokinase (glk) or a homolog thereof, (iii) phosphoglucomutase (pgm) or a homolog thereof, (iv) UTP-glucose-1-phosphate-uridylyltransferase (galU) or a homolog thereof, (v) glucose-6-phopshate 1-dehydrogenase (zwf) or a homolog thereof, (vi) 6-phosphogluconate dehydrogenase (gnd) or a homolog thereof, (vii) phosphoribosylpyrophosphate synthetase (prs) or a homolog thereof; and (viii) any combinations thereof. In certain embodiments, the one or more genetic modifications is selected from a group consisting of: (i) overexpression of glucokinase (glk) or a homolog thereof, (ii) overexpression of glucose facilitator gene (glf) or a homolog thereof; and (iii) any combinations thereof.

In certain embodiments, the one or more genetic modifications is overexpression of phosphoglucomutase (pgm) or a homolog thereof. In certain embodiments, the one or more genetic modifications is overexpression of UTP-glucose-1-phosphate-uridylyltransferase (galU) or a homolog thereof. In certain embodiments, one or more genetic modifications is overexpression of orotate phosphoribosyltransferase (pyrE) or a homolog thereof. In certain embodiments, one or more genetic modifications is overexpression of orotidine 5′phosphate decarboxylase (pyrF), or a homolog thereof. In certain embodiments, the engineered cells are supplemented with a medium comprising orotic acid. In certain embodiments, the one or more genetic modifications is overexpression of uridylate kinase (pyrH) or a homolog thereof. In certain embodiments, the one or more genetic modifications is overexpression of nucleoside diphosphate kinase (ndk) or a homolog thereof. In certain embodiments, the one or more genetic modifications is overexpression of adenylate kinase (adk) or a homolog thereof. In certain embodiments, the one or more genetic modifications is overexpression of fructokinase (cscK). In certain embodiments, the overexpression of fructokinase (cscK) leads to phosphorylation of fructose to generate fructose-6-phosphate. In certain embodiments, the one or more genetic modifications is downregulation or deletion of UDP-glucose 4-epimerase (galE) to prevent generation of UDP-galactose. In certain embodiments, the one or more genetic modifications is downregulation or deletion of UDP-glucose 6-dehydrogenase (ugd) to prevent conversion of UDP-glucose to UDP-glucuronate. In certain embodiments, the one or more genetic modifications is downregulation or deletion of glucose-1-phosphate adenylyltransferase (glgC). In certain embodiments, the one or more genetic modifications is downregulation or deletion of glucans biosynthesis glucosyltransferase (OpgG) to prevent consumption of UDP-glucose to form membrane-derived oligosaccharides (MDO). In certain embodiments, the one or more genetic modifications is downregulation or deletion of glucose-6-phosphate isomerase (pgi). In certain embodiments, the one or more genetic modifications is overexpression of glucose-6-phopshate 1-dehydrogenase (zwf) or a homolog thereof. In certain embodiments, the one or more genetic modifications is overexpression of 6-phosphogluconate dehydrogenase (gnd) or a homolog thereof. In certain embodiments, the one or more genetic modifications is overexpression of phosphoribosylpyrophosphate synthetase (prs) or a homolog thereof. In certain embodiments, the one or more genetic modifications is overexpression of a sucrose transporter (cscB) or a homolog thereof. In certain embodiments, the one or more genetic modifications is overexpression of sucrose synthase (SuSy) or a homolog thereof. In certain embodiments, the one or more genetic modifications is overexpression of sucrose phosphorylase (SPase) or a homolog thereof. In certain embodiments, the engineered host cells are supplemented with a medium comprising glucose. In certain embodiments, the one or more genetic modifications is deletion or downregulation of UDP-sugar hydrolase (ushA). In certain embodiments, the one or more genetic modifications is deletion or downregulation of UTP-glucose-1-phosphate uridylyltransferase (ugp). In certain embodiments, the one or more genetic modification is downregulation or deletion of (heptosyl) lipopolysaccharide α-1,3-glucosyltransferase (WaaG). (heptosyl) lipopolysaccharide α-1,3-glucosyltransferase (WaaG) uses UDP-glucose as a substrate for lipopolysaccharide synthesis. Thus, in certain embodiments, downregulation of (heptosyl) lipopolysaccharide α-1,3-glucosyltransferase (WaaG) enhance the yield of UDP-glucose. In certain embodiments, the one or more genetic modification is downregulation or deletion of trehalose-6-phosphate synthase (otsA). Trehalose-6-phosphate synthase (otsA) catalyzes the biosynthesis of trehalose from UDP-glucose. Thus, in certain embodiments, downregulation of trehalose-6-phosphate synthase (otsA) will enhance the yield of UDP-glucose. In certain embodiments, the one or more genetic modification is downregulation or deletion of glucose-1-phosphatase (agp). Glucose-1-phosphatase (agp) is a periplasmic protein that hydrolyses phosphate from glucose-1-phosphate and other substrates. Thus, in certain embodiments, downregulation or deletion of glucose-1-phosphatase (agp) will increase the availability of glucose-1-phosphate for UDP-glucose production, and enhancing the yield of UDP-sugar.

In certain embodiments, the engineered host cells are supplemented with a medium comprising glucose. In certain embodiments, the engineered host cells are supplemented with a medium comprising sucrose. In certain embodiments, the engineered host cells are supplemented with a medium comprising fructose.

In another aspect, the invention provides an engineered host cell that comprises one or more genetic modifications that result in enzymatic transformation by the engineered host cell of glucose through multiple chemical intermediates into uridine diphosphate galactose (UDP-galactose). In certain embodiments, the engineered host cell is E. coli. In certain embodiments, the one or more genetic modifications are at least one genetic modification selected from the group consisting of: (i) one or more modifications for over-expressing one or more endogenous genes in the engineered host cells; (ii) one or more modifications for under-expressing one or more endogenous genes in the engineered host cells; (iii) one or more genetic modification is expressing one or more non-native genes in the engineered host cells; and (iv) a combination thereof. In certain embodiments, the one or more genetic modification is selected from a group consisting of: (i) overexpression of UDP-glucose 4-epimerase (galE) or a homolog thereof, (ii) overexpression of galactokinase (galK) or a homolog thereof, (iii) overexpression of galactose-1-phosphate uridylyltransferase (galT) or a homolog thereof, and (iv) any combinations thereof. In certain embodiments, the one or more genetic modifications is overexpression of UDP-glucose 4-epimerase (galE) or a homolog thereof. In certain embodiments, the one or more genetic modifications is overexpression of galactokinase (galK) or a homolog thereof. In certain embodiments, the one or more genetic modifications is overexpression of galactose-1-phosphate uridylyltransferase (galT) or a homolog thereof. In certain embodiments, the modifications listed herein may be combined with modifications listed in any other aspects of the invention. In certain embodiments, the modifications listed herein may be combined with one or more modifications listed for the transformation of chemical intermediates into UDP-glucose. In certain embodiments, the engineered host cells are supplemented with a medium comprising galactose. In certain embodiments, the engineered host cells are supplemented with a medium comprising fructose.

In another aspect, the invention provides an engineered host cell that comprises one or more genetic modifications that result in enzymatic transformation by the engineered host cell of sucrose into uridine diphosphate glucose (UDP-glucose). In certain embodiments, the genetic modifications are selected from a group consisting of: (i) heterologous expression of a sucrose transporter (cscB) or a homolog thereof, (ii) heterologous expression of sucrose synthase (SuSy) or a homolog thereof, (iii) downregulation or deletion of UDP-glucose 4-epimerase (galE), (iv) downregulation or deletion of UDP-glucose 6-dehydrogenase (ugd), (v) downregulation or deletion of glucans biosynthesis glucosyltransferase (OpgG), and (vi) any combinations thereof. In certain embodiments, the engineered cell comprises heterologous sucrose transporter (cscB) or a homolog thereof, heterologous sucrose synthase (SuSy) or a homolog thereof, and genetic modifications selected from a group consisting of: (i) downregulation or deletion of UDP-glucose 4-epimerase (galE), (ii) downregulation or deletion of UDP-glucose 6-dehydrogenase (ugd), (iii) downregulation or deletion of glucans biosynthesis glucosyltransferase (OpgG), and (iv) any combinations thereof. In certain embodiments, the genetic modifications are selected from a group consisting of: (i) expression of phosphoribosyltransferase (pyrE) or a homolog thereof, (ii) expression of orotidine 5′-phosphate decarboxylase (pyrF) or a homolog thereof, (iii) expression of uridylate kinase (pyrH) or a homolog thereof, (iv) expression of nucleoside diphosphate kinase (ndk) or a homolog thereof, (v) expression of adenylate kinase (adk) or a homolog thereof, and (vi) any combination thereof. In certain embodiments, the modifications listed herein may be combined with modifications listed in any other aspects of the invention.

In another aspect, the invention provides an engineered host cell that comprises one or more genetic modifications that result in enzymatic transformation by the engineered host cell of sucrose into uridine diphosphate glucose (UDP-glucose). In certain embodiments, the genetic modifications are selected from a group consisting of: (i) heterologous expression of a sucrose transporter (cscB) or a homolog thereof, (ii) heterologous expression of sucrose phosphorylase (SPase) or a homolog thereof, (iii) overexpression of UTP-glucose-1-phosphate-uridylyltransferase (galU) or a homolog thereof, (iv) downregulation or deletion of UDP-glucose 4-epimerase (galE), (v) downregulation or deletion of UDP-glucose 6-dehydrogenase (ugd), (vi) downregulation or deletion of glucans biosynthesis glucosyltransferase (OpgG), (vii) downregulation or deletion of phosphoglucomutase (pgm), and (viii) any combinations thereof. In certain embodiments, the engineered cell comprises heterologous sucrose transporter (cscB) or a homolog thereof, heterologous sucrose phosphorylase (SPase) or a homolog thereof, overexpression of UTP-glucose-1-phosphate-uridylyltransferase (galU) or a homolog thereof, and genetic modifications selected from a group consisting of: (i) downregulation or deletion of UDP-glucose 4-epimerase (galE), (ii) downregulation or deletion of UDP-glucose 6-dehydrogenase (ugd), (iii) downregulation or deletion of glucans biosynthesis glucosyltransferase (OpgG), and (iv) any combinations thereof. In certain embodiments, the genetic modifications are selected from a group consisting of: (i) expression of phosphoribosyltransferase (pyrE) or a homolog thereof, (ii) expression of orotidine 5′-phosphate decarboxylase (pyrF) or a homolog thereof, (iii) expression of uridylate kinase (pyrH) or a homolog thereof, (iv) expression of nucleoside diphosphate kinase (ndk) or a homolog thereof, (v) expression of adenylate kinase (adk) or a homolog thereof, and (vi) any combination thereof. In certain embodiments, the modifications listed herein may be combined with modifications listed in any other aspects of the invention.

In another aspect, the invention provides a method of increasing the production of uridine diphosphate glucose (UDP-glucose), the method comprising: providing an engineered host cell that comprises one or more genetic modifications that result in enzymatic transformation by the engineered host cell of glucose through multiple chemical intermediates into UDP-glucose. In certain embodiments, the one or more genetic modifications are at least one genetic modification selected from the group consisting of: (i) one or more modifications for over-expressing one or more endogenous genes in the engineered host cells; (ii) one or more modifications for under-expressing one or more endogenous genes in the engineered host cells; (iii) one or more genetic modification is expressing one or more non-native genes in the engineered host cells; and (iv) a combination thereof. In certain embodiments, the engineered host cell is E. coli. In certain embodiments, the one or more genetic modifications is overexpression of one or more genes selected from a group consisting of: (i) glucokinase (glk) or a homolog thereof, (ii) glucose facilitator gene (glf) or a homolog thereof, (iii) phosphoglucomutase (pgm) or a homolog thereof, (iv) UTP-glucose-1-phosphate-uridylyltransferase (galU) or a homolog thereof, (v) phosphoribosyltransferase (pyrE) or a homolog thereof, (vi) orotidine 5′phosphate decarboxylase (pyrF) or a homolog thereof, (vii) uridylate kinase (pyrH) or a homolog thereof, (viii) nucleoside diphosphate kinase (ndk) or a homolog thereof, (ix) adenylate kinase (adk) or a homolog thereof, (x) fructokinase (cscK) or a homolog thereof; and (xi) any combinations thereof. In certain embodiments, the one or more genetic modifications is overexpression of one or more genes selected from a group consisting of: (i) glucokinase (glk) or a homolog thereof, (ii) glucose facilitator gene (glf) or a homolog thereof, (iii) phosphoglucomutase (pgm) or a homolog thereof, (iv) UTP-glucose-1-phosphate-uridylyltransferase (galU) or a homolog thereof, (v) glucose-6-phopshate 1-dehydrogenase (zwf) or a homolog thereof, (vi) 6-phosphogluconate dehydrogenase (gnd) or a homolog thereof, (vii) phosphoribosylpyrophosphate synthetase (prs); and (viii) any combinations thereof. In certain embodiments, the one or more genetic modifications is selected from a group consisting of: (i) overexpression of glucokinase (glk) or a homolog thereof, (ii) overexpression of glucose facilitator gene (glf) or a homolog thereof; and (iii) any combinations thereof. In certain embodiments, the one or more genetic modifications is overexpression of phosphoglucomutase (pgm) or a homolog thereof. In certain embodiments, the one or more genetic modifications is overexpression of UTP-glucose-1-phosphate-uridylyltransferase (galU) or a homolog thereof. In certain embodiments, one or more genetic modifications is overexpression of fructokinase (cscK) or a homolog thereof. In certain embodiments, one or more genetic modifications is overexpression of orotate phosphoribosyltransferase (pyrE). In certain embodiments, one or more genetic modifications is overexpression of orotidine 5′phosphate decarboxylase (pyrF). In certain embodiments, the engineered host cells are supplemented with a medium comprising orotic acid. In certain embodiments, the one or more genetic modifications is overexpression of uridylate kinase (pyrH). In certain embodiments, the one or more genetic modifications is overexpression of nucleoside diphosphate kinase (ndk). In certain embodiments, the one or more genetic modifications is overexpression of adenylate kinase (adk). In certain embodiments, the one or more genetic modifications is downregulation or deletion of UDP-glucose 4-epimerase (galE) to prevent conversion of UDP-glucose to UDP-galactose. In certain embodiments, the one or more genetic modifications is downregulation or deletion of UDP-glucose 6-dehydrogenase (ugd) to prevent conversion of UDP-glucose to UDP-glucuronate. In certain embodiments, the one or more genetic modifications is downregulation or deletion of glucose-1-phosphate adenylyltransferase (glgC). In certain embodiments, the one or more genetic modifications is downregulation or deletion of glucans biosynthesis glucosyltransferase (mdoA) to prevent conversion of UDP-glucose to membrane-derived oligosaccharides (MDO). In certain embodiments, the one or more genetic modifications is downregulation or deletion of glucose-6-phosphate isomerase (pgi). In certain embodiments, the one or more genetic modification is downregulation or deletion of (heptosyl) lipopolysaccharide α-1,3-glucosyltransferase (WaaG). (heptosyl) lipopolysaccharide α-1,3-glucosyltransferase (WaaG) uses UDP-glucose as a substrate for lipopolysaccharide synthesis. Thus, in certain embodiments, downregulation of (heptosyl) lipopolysaccharide α-1,3-glucosyltransferase (WaaG) enhance the yield of UDP-glucose. In certain embodiments, the one or more genetic modification is downregulation or deletion of trehalose-6-phosphate synthase (otsA). Trehalose-6-phosphate synthase (otsA) catalyzes the biosynthesis of trehalose from UDP-glucose. Thus, in certain embodiments, downregulation of trehalose-6-phosphate synthase (otsA) will enhance the yield of UDP-glucose. In certain embodiments, the one or more genetic modification is downregulation or deletion of glucose-1-phosphatase (agp). Glucose-1-phosphatase (agp) is a periplasmic protein that hydrolyses phosphate from glucose-1-phosphate and other substrates. Thus, in certain embodiments, downregulation or deletion of glucose-1-phosphatase (agp) will increase the availability of glucose-1-phosphate for UDP-glucose production, and enhancing the yield of UDP-sugar. In certain embodiments, the one or more genetic modifications is overexpression of glucose-6-phopshate 1-dehydrogenase (zwf) or a homolog thereof. In certain embodiments, the one or more genetic modifications is overexpression of 6-phosphogluconate dehydrogenase (gnd) or a homolog thereof. In certain embodiments, the one or more genetic modifications is overexpression of phosphoribosylpyrophosphate synthetase (prs) or a homolog thereof. In certain embodiments, wherein the engineered host cells include overexpression of: (i) glucokinase (glk) or a homolog thereof, (ii) glucose facilitator gene (glf) or a homolog thereof, (iii) phosphoglucomutase (pgm) or a homolog thereof, (iv) UTP-glucose-1-phosphate-uridylyltransferase (galU) or a homolog thereof, (v) phosphoribosyltransferase (pyrE) or a homolog thereof, (vi) orotidine 5′phosphate decarboxylase (pyrF) or a homolog thereof, (vii) uridylate kinase (pyrH) or a homolog thereof, (viii) nucleoside diphosphate kinase (ndk) or a homolog thereof, (ix) adenylate kinase (adk) or a homolog thereof; (x) fructokinase (cscK) or a homolog thereof, and (xi) any combinations thereof. In certain embodiments, the engineered host cells include overexpression of: (i) glucokinase (glk) or a homolog thereof, (ii) glucose facilitator gene (glf) or a homolog thereof, (iii) phosphoglucomutase (pgm) or a homolog thereof, (iv) UTP-glucose-1-phosphate-uridylyltransferase (galU) or a homolog thereof, (v) glucose-6-phopshate 1-dehydrogenase (zwf) or a homolog thereof, (vi) 6-phosphogluconate dehydrogenase (gnd) or a homolog thereof, and (vii) phosphoribosylpyrophosphate synthetase (prs) or a homolog thereof. In another aspect, the invention provides a method of increasing production of uridine diphosphate galactose (UDP-galactose), the method comprising: providing an engineered host cell that comprises one or more genetic modifications that result in enzymatic transformation by the engineered host cell of glucose and/or galactose through multiple chemical intermediates into uridine diphosphate galactose (UDP-galactose). In certain embodiments, the engineered host cell is E. coli. In certain embodiments, the one or more genetic modifications are at least one genetic modification selected from the group consisting of: (i) one or more modifications for over-expressing one or more endogenous genes in the engineered host cells; (ii) one or more modifications for under-expressing one or more endogenous genes in the engineered host cells; (iii) one or more genetic modification is expressing one or more non-native genes in the engineered host cells; and (iv) a combination thereof. In certain embodiments, the one or more genetic modification is selected from a group consisting of: (i) overexpression of UDP-glucose 4-epimerase (galE) or a homolog thereof, (ii) overexpression of galactokinase (galK) or a homolog thereof, (iii) overexpression of galactose-1-phosphate uridylyltransferase (galT) or a homolog thereof, and (iv) any combinations thereof. In certain embodiments, the one or more genetic modifications is overexpression of UDP-glucose 4-epimerase (galE) or a homolog thereof. In certain embodiments, the one or more genetic modifications is overexpression of galactokinase (galK) or a homolog thereof. In certain embodiments, the one or more genetic modifications is overexpression of hydroxyproline O-galactosyltransferase (galT) or a homolog thereof. In certain embodiments, the genetic modifications are selected from a group consisting of: (i) expression of phosphoribosyltransferase (pyrE) or a homolog thereof, (ii) expression of orotidine 5′-phosphate decarboxylase (pyrF) or a homolog thereof, (iii) expression of uridylate kinase (pyrH) or a homolog thereof, and (iv) any combination thereof. In certain embodiments, the modifications listed herein may be combined with modifications listed in any other aspects of the invention. In certain embodiments, the engineered host cells are supplemented with a medium comprising galactose.

In another aspect, the invention provides a method for increasing the production of UDP-glucose, the method comprising: providing an engineered host cell that comprises one or more genetic modifications that result in enzymatic transformation by the engineered host cell of sucrose into uridine diphosphate glucose (UDP-glucose). In certain embodiments, the genetic modifications are selected from a group consisting of: (i) heterologous expression of a sucrose transporter (cscB) or a homolog thereof, (ii) heterologous expression of sucrose phosphorylase (SPase) or a homolog thereof, (iii) overexpression of UTP-glucose-1-phosphate-uridylyltransferase (galU) or a homolog thereof, (iv) downregulation or deletion of UDP-glucose 4-epimerase (galE), (v) downregulation or deletion of UDP-glucose 6-dehydrogenase (ugd), (vi) downregulation or deletion of glucans biosynthesis glucosyltransferase (OpgG), (vii) downregulation or deletion of phosphoglucomutase (pgm), and (viii) any combinations thereof. In certain embodiments, the engineered cell comprises heterologous sucrose transporter (cscB) or a homolog thereof, heterologous sucrose phosphorylase (SPase) or a homolog thereof, overexpression of UTP-glucose-1-phosphate-uridylyltransferase (galU) or a homolog thereof, and genetic modifications selected from a group consisting of: (i) downregulation or deletion of UDP-glucose 4-epimerase (galE), (ii) downregulation or deletion of UDP-glucose 6-dehydrogenase (ugd), (iii) downregulation or deletion of glucans biosynthesis glucosyltransferase (OpgG), and (iv) any combinations thereof. In certain embodiments, the genetic modifications are selected from a group consisting of: (i) expression of phosphoribosyltransferase (pyrE) or a homolog thereof, (ii) expression of orotidine 5′-phosphate decarboxylase (pyrF) or a homolog thereof, (iii) expression of uridylate kinase (pyrH) or a homolog thereof, (iv) expression of nucleoside diphosphate kinase (ndk) or a homolog thereof, (v) expression of adenylate kinase (adk) or a homolog thereof, and (vi) any combination thereof. In certain embodiments, the modifications listed herein may be combined with modifications listed in any other aspects of the invention.

In another aspect, the invention provides a method of increasing the production of uridine diphosphate glucose (UDP-glucose) from sucrose, the method comprising: providing an engineered host cell that comprises one or more genetic modifications that result in enzymatic transformation by the engineered host cell of sucrose into UDP-glucose. In certain embodiments, the genetic modifications are selected from a group consisting of: (i) heterologous expression of a sucrose transporter (cscB) or a homolog thereof, (ii) heterologous expression of sucrose synthase (SuSy) or a homolog thereof, (iii) downregulation or deletion of UDP-glucose 4-epimerase (galE), (iv) downregulation or deletion of UDP-glucose 6-dehydrogenase (ugd), (v) downregulation or deletion of glucans biosynthesis glucosyltransferase (OpgG), and (vi) any combinations thereof. In certain embodiments, the engineered cell comprises heterologous sucrose transporter (cscB) or a homolog thereof, heterologous sucrose synthase (SuSy) or a homolog thereof, and genetic modifications selected from a group consisting of: (i) downregulation or deletion of UDP-glucose 4-epimerase (galE), (ii) downregulation or deletion of UDP-glucose 6-dehydrogenase (ugd) or a homolog thereof, (iii) downregulation or deletion of glucans biosynthesis glucosyltransferase (OpgG), and (iv) any combinations thereof. In certain embodiments, the engineered cell comprises one more genetic modifications are selected from a group consisting of: (i) expression of phosphoribosyltransferase (pyrE) or a homolog thereof, (ii) expression of orotidine 5′-phosphate decarboxylase (pyrF) or a homolog thereof, (iii) expression of uridylate kinase (pyrH) or a homolog thereof, and (iv) any combinations thereof. In certain embodiments, the engineered cells are supplemented with a medium comprising orotic acid, sucrose, fructose, and/or glucose.

### V. BRIEF DESCRIPTION OF THE DRAWING(S)

FIG. 1 shows the pathways of UDP-glucose and/or UDP-galactose bioproduction in engineered cells and methods of preparing UDP-glucose and/or UDP-galactose described herein.

FIG. 2 provides genetic modifications in the engineered host cell for increasing production of UDP-glucose and/or UDP-galactose.

### VI. DETAILED DESCRIPTION OF THE INVENTION

The present application provides engineered cells for producing UDP-sugar including, but not limited to UDP-glucose and/or UDP-galactose, cultures that include the engineered cells, and methods of producing UDP-sugars including, but not limited to UDP-glucose and/or UDP-galactose. The terms ‘precursor’ as used herein may refer to any intermediate present in the biosynthetic pathway that leads to the production of UDP-glucose and/or UDP-galactose. UDP-sugars precursors may include, but are not limited to glucose, galactose, sucrose, fructose-6-phosphate (F6P), glucose-6-phosphate (G6P), 6-phosphogluconate, ribose-5-phosphate (R5P), 5-phosphoribosyl 1-pyrophosphate (PRPP), Orotidine 5′-phosphate (OMP), uridine monophosphate (UMP), uridine diphosphate (UDP), uridine triphosphate (UTP), glucose-1-phosphate (Glucose-1-P), galactose-1-phosphate (Galactose-1-P), uridine diphosphate glucuronate (UDP-glucuronate), orotic acid, and ADP-α-D-glucose.

Cells engineered for the production of UDP-sugars can have one or multiple modifications, including, without limitation, the downregulation, disruption, or deletion of endogenous genes, the upregulation of an endogenous gene, and the introduction of exogenous genes.

The term “non-naturally occurring”, when used in reference to an enzyme is intended to mean that nucleic acids or polypeptides include at least one genetic alteration not normally found in a naturally occurring polypeptide or nucleic acid sequence. Naturally occurring nucleic acids, and polypeptides can be referred to as “wild-type” or “original”. A host cell, organism, or microorganism that includes at least one genetic modification generated by human intervention can also be referred to as “non-naturally occurring”, “engineered”, “genetically engineered,” or “recombinant”.

A host cell, organism, or microorganism engineered to express or overexpress a gene or nucleic acid sequence, or to overexpress an enzyme or polypeptide has been genetically engineered through recombinant DNA technology to include a gene or nucleic acid sequence that does not naturally encode the enzyme or polypeptide or to express an endogenous gene at a level that exceeds its level of expression in a non-altered cell. As nonlimiting examples, a host cell, organism, or microorganism engineered to express or overexpress a gene or a nucleic acid sequence, or to overexpress an enzyme or polypeptide can have any modifications that affect a coding sequence of a gene, the position of a gene on a chromosome or regulatory elements associated with a gene. Overexpression of a gene can also be by increasing the copy number of a gene in the cell or organism. Similarly, a host cell, organism, or microorganism engineered to under-express or to have reduced expression of a gene, nucleic acid sequence, or to under-express an enzyme or polypeptide can have any modifications that affect a coding sequence of a gene, the position of a gene on a chromosome or regulatory elements associated with a gene. Specifically included are gene disruptions, which include any insertions, deletions, or sequence mutations into or of the gene or a portion of the gene that affect its expression or the activity of the encoded polypeptide. Gene disruptions include “knockout” mutations that eliminate expression of the gene. Modifications to under-express a gene also include modifications to regulatory regions of the gene that can reduce its expression.

The term “exogenous” or “heterologous” is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material that may be introduced on a vehicle such as a plasmid. Therefore, the term “endogenous” refers to a referenced molecule or activity that is naturally present in the host.

Genes or nucleic acid sequences can be introduced stably or transiently into a host cell using techniques well known in the art including, but not limited to, conjugation, electroporation, chemical transformation, transduction, and transfection. Optionally, for exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. Furthermore, genes can be subjected to codon optimization with techniques well known in the art to achieve optimized expression of the proteins.

The percent identity (% identity) between two sequences is determined when sequences are aligned for maximum homology. Algorithms well known to those skilled in the art, such as Align, BLAST, Clustal Omega, and others compare and determine a raw sequence similarity or identity, and also determine the presence or significance of gaps in the sequence which can be assigned a weight or score. Such algorithms also are known in the art and are similarly applicable for determining nucleotide or amino acid sequence similarity or identity and can be useful in identifying orthologs of genes of interest. Additional sequences added to a polypeptide sequence, such as but not limited to immunodetection tags, purification tags, localization sequences (presence or absence), etc., do not affect the % identity.

A homolog is a gene or genes that have the same or identical functions in different organisms. Genes that are orthologous can encode proteins with sequence similarity of about 45% to 100% amino acid sequence identity, and more preferably about 60% to 100% amino acid sequence identity. Genes can be considered orthologs if they share three-dimensional structure but not necessarily sequence similarity, of a sufficient amount to indicate that they have evolved from a common ancestor to the extent that the primary sequence similarity is not identifiable. Paralogs are genes related by duplication within a genome, and can evolve new functions, even if these are related to the original one.

An engineered cell for producing UDP-glucose can include an exogenous nucleic acid sequence encoding glucose facilitator (glf) activity or a homolog thereof, an exogenous nucleic acid sequence encoding glucokinase (glk) activity or a homolog thereof, an exogenous nucleic acid sequence encoding phosphoglucomutase (pgm) activity or a homolog thereof, an exogenous nucleic acid sequence encoding UTP-glucose-1-phosphate-uridylyltransferase (galU) activity or a homolog thereof, and an exogenous nucleic acid sequence encoding phosphoribosyltransferase (pyrE) activity, orotidine 5′-phosphate decarboxylase (pyrF) or a homolog thereof, an exogenous nucleic acid sequence encoding uridylate kinase (pyrH) activity or a homolog thereof, an exogenous nucleic acid sequence encoding nucleoside diphosphate kinase (ndk) activity or a homolog thereof, an exogenous nucleic acid sequence encoding adenylate kinase (adk) activity or a homolog thereof, an exogenous nucleic acid sequence encoding glucose-6-phopshate 1-dehydrogenase (zwf) activity or a homolog thereof, an exogenous nucleic acid sequence encoding 6-phosphogluconate dehydrogenase (gnd) activity or a homolog thereof, an exogenous nucleic acid sequence encoding phosphoribosylpyrophosphate synthetase (prs) activity or a homolog thereof, an exogenous nucleic acid sequence encoding sucrose transporter (cscB) activity or a homolog thereof, an exogenous expression nucleic acid sequence encoding sucrose synthase (SuSy) activity or a homolog thereof, and an exogenous expression nucleic acid sequence encoding sucrose phosphorylase (SPase) activity or a homolog thereof. Optionally, the engineered cell can further include downregulation or deletion of UDP-glucose 4-epimerase (galE) activity, downregulation or deletion of UDP-glucose 6-dehydrogenase (ugd) activity, downregulation or deletion of glucose-1-phosphate adenylyltransferase (glgC) activity, downregulation or deletion of glucosyltransferase (OpgG) activity, downregulation or deletion of glucose-6-phosphate isomerase (pgi) activity, downregulation or deletion of UDP-sugar hydrolase (ushA) activity, downregulation or deletion of UTP-glucose-1-phosphate uridylyltransferase (ugp) activity, and downregulation or deletion of phosphoglucomutase (pgm) activity.

Glucose facilitator gene or glucose transporter gene (glf) can be, for example, a member of the major facilitator superfamily or sugar transporter (TC 2.A.1.1) that facilitates glucose uptake by cells An exemplary glucose facilitator gene can be glf from Zymomonas mobilis (NCBI Accession ID: AAA27691.1) Also considered for use in the engineered cells provided herein are glf homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID NO: 1 that have the activity of transporting glucose into cells.

Glucokinase (glk) can be, for example, a member of the hexokinase family of proteins that phosphorylate glucose to produce glucose-6-phosphate. An exemplary glucokinase (glk) is the Zymomonas mobilis (EC: 2.7.1.2, NCBI Ref: AAA27694.1). Also considered for use in the engineered cells provided herein are glk homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID NO: 2 that have the activity of a glucokinase that produces glucose-6-phosphate from glucose.

Phosphoglucomutase (pgm) catalyzes the conversion of glucose 6-phosphate to glucose 1-phosphate. An exemplary phosphoglucomutase (pgm) can be pgm from Escherichia coli (EC: 5.4.2.2, NCBI Accession AAC73782.1). Also considered for use in the engineered cells provided herein are pgm with SEQ ID NOS: 4-22, pgm homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID NOS: 3-22 that have the activity of catalyzing the conversion of glucose 6-phosphate to glucose 1-phosphate.

UTP-glucose-1-phosphate-uridylyltransferase (galU) is an enzyme that synthesizes UDP-glucose from glucose-1-phosphate and UTP. An exemplary UTP-glucose-1-phosphate-uridylyltransferase (galU) can be galU from Escherichia coli (EC: 2.7.7.9, NCBI Accession EEW2752841.1). Also considered for use in the engineered cells provided herein are galU with SEQ ID NOS: 23-39 or 72-74. Also considered for use in the engineered cells provided herein are ugpA from Bifidobacterium bifidum (NCBI Accession WP_021648042.1). Also considered for use in the engineered cells provided herein are ugpA with SEQ ID NOS: 41-47. Also considered for use in the engineered cells provided herein are galU or ugpA homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID NOS: 23-47 or 72-74 that have the activity of synthesis UDP-glucose from glucose-1-phosphate.

Orotate phosphoribosyltransferase (pyrE) is an enzyme that catalyzes the transfer of a ribosyl phosphate group from 5-phosphoribose 1-diphosphate to orotate, leading to the formation of orotidine monophosphate (OMP). An exemplary Orotate phosphoribosyltransferase can be pyrE from Enterobacteriaceae (NCBI Accession WP_000806177.1). Also considered for use in the engineered cells provided herein are pyrE with SEQ ID NO: 49, pyrE homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID NO: 48-49 that have the activity of catalyzing phosphoribose-1-diphosphate (PRPP) to orotidine monophosphate (OMP).

Orotidine-5′-phosphate decarboxylase (pyrF) is an enzyme that catalyzes the conversion of orotidine monophosphate (OMP) to uridine monophosphate (UMP). An exemplary orotidine-5′-phosphate decarboxylase (pyrF) can be pyrF from Escherichia coli, (EC: 2.4.2.10, NCBI Accession WP_110991478.1). Also considered for use in the engineered cells provided herein are pyrF with SEQ ID NO: 51, pyrF homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID NOS: 50-51 that have the activity of catalyzing orotidine monophosphate (OMP) to uridine monophosphate (UMP).

Uridylate kinase (pyrH) is an enzyme that catalyzes the conversion of uridine monophosphate (UMP) to uridine diphosphate (UDP). An exemplary uridylate kinase (pyrH) can be pyrH from Escherichia coli (EC: 2.7.4.22, NCBI Accession WP_000224573.1). Also considered for use in the engineered cells provided herein are pyrH with SEQ ID NO: 53, pyrH homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID NOS: 52-53 that have the activity of catalyzing the conversion of uridine monophosphate (UMP) to uridine diphosphate (UDP).

Nucleoside diphosphate kinase (ndk) is an enzyme that catalyzes the conversion of uridine diphosphate (UDP) to uridine triphosphate (UTP). An exemplary nucleoside diphosphate kinase (ndk) can be ndk from Enterobacteriaceae (EC: 2.7.4.6, NCBI Accession WP_000963837.1). Also considered for use in the engineered cells provided herein are ndk with SEQ ID NO: 55, ndk homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID NO: 54-55 that have the activity of catalyzing the conversion of uridine diphosphate (UDP) to uridine triphosphate (UTP).

Adenylate kinase (adk) is an enzyme that catalyzes the interconversion of the various adenosine phosphates (ATP, ADP, and AMP). An exemplary adenylate kinase (adk) can be adk from Escherichia coli (EC: 2.7.4.3, NCBI Accession AAC73576.1). Also considered for use in the engineered cells provided herein are adk with SEQ ID NO: 57, adk homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID NOS: 56-57 that have the activity of catalyzing the interconversion of the various adenosine phosphates (ATP, ADP, and AMP).

Glucose-6-phopshate 1-dehydrogenase (zwf) is an enzyme that catalyzes oxidation of glucose 6-phosphate to 6-phosphogluconolactone. An exemplary glucose-6-phopshate 1-dehydrogenase (zwf) can be zwf from Escherichia coli (EC: 1.1.1.49, NCBI Accession UGE34297.1). Also considered for use in the engineered cells provided herein are zwf homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID NO: 58 that have the activity of catalyzing the oxidation of glucose 6-phosphate to 6-phosphogluconolactone.

6-phosphogluconate dehydrogenase (gnd) is an enzyme that catalyzes the oxidative decarboxylation of 6-phosphogluconate to ribose 5-phosphate and CO₂, with concomitant reduction of NADP to NADPH. An exemplary 6-phosphogluconate dehydrogenase (gnd) can be gnd from Enterobacteriaceae (EC: 1.1.1.44, NCBI Accession WP_000043484.1). Also considered for use in the engineered cells provided herein are gnd homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID NO: 59 that have the activity of catalyzing the oxidative decarboxylation of 6-phosphogluconate to ribose-5-phosphate and CO₂, with concomitant reduction of NADP to NADPH.

PRPP synthase (prs) is an enzyme that catalyzes the conversion of ribose-5-phosphate to PRPP via the transfer of pyrophosphoryl group from ATP to PRPP. An exemplary PRPP synthase (prs) can be prs from Enterobacteriaceae (EC: 2.7.6.1, NCBI Accession WP_001298109.1). Also considered for use in the engineered cells provided herein are prs homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID NO: 60 that have the activity of catalyzing the conversion of ribose-5-phosphate to PRPP via the transfer of pyrophosphoryl group from ATP to PRPP.

Sucrose permease (cscB) is responsible for transport of sucrose into the cells. An exemplary sucrose permease (cscB) can be cscB from Enterobacteriaceae (EC: 2.7.1.69, NCBI Accession WP_001197025.1). Also considered for use in the engineered cells provided herein are cscB homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID NO: 61 that have the activity of transporting sucrose in the cells.

Sucrose synthase (SuSy) catalyzes the reversible conversion of sucrose and a nucleoside diphosphate into fructose and nucleotide (NDP)-glucose. An exemplary sucrose synthase (SuSy) can be SuSy from Acidithiobacillus caldus (E.C. 2.4.1.13, NCBI Accession WP_004872341.1). Also considered for use in the engineered cells provided herein are SuSy homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID NO: 62 that have the activity of reversible conversion of sucrose and a nucleoside diphosphate into fructose and nucleotide (NDP)-glucose.

Sucrose phosphorylase (SPase) catalyzes the reversible phosphorolysis of sucrose into alpha-D-glucose 1-phosphate (Glc1P) and D-fructose. An exemplary sucrose phosphorylase can be sucP from Bifidobacterium adolescentis (E.C. 2.4.1.7, NCBI Accession WP_011742626.1). Also considered for use in the engineered cells provided herein are SPase homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID NO: 63 that have the activity of reversible phosphorolysis of sucrose into alpha-D-glucose 1-phosphate (Glc1P) and D-fructose.

UDP-glucose 4-epimerase (galE) is an enzyme that catalyzes the reversible conversion of UDP-galactose to UDP-glucose via the through a mechanism involving the transient reduction of NAD. An exemplary UDP-glucose 4-epimerase (galE) can be galE from Escherichia coli (EC: 5.1.3.2, NCBI Accession AAV80748.1). Also considered for overexpression, downregulation, or deletion in the engineered cells provided herein are galE homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID NO: 64 that have the activity of catalyzing the reversible conversion of UDP-galactose to UDP-glucose via the through a mechanism involving the transient reduction of NAD.

UDP-glucose 6-dehydrogenase (ugd) is an enzyme catalyzes the conversion of UDP-glucose to UDP-glucuronate. An exemplary UDP-glucose 6-dehydrogenase (ugd) can be ugd from Escherichia coli (EC: 1.1.1.22, NCBI Accession WP_089615770.1). Also considered for downregulation or deletion in the engineered cells provided herein are ugd homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID NO: 65 that have the activity of catalyzing the reversible conversion of UDP-glucose to UDP-glucuronate.

Glucose-1-phosphate adenylyltransferase (glgC) is an enzyme that catalyzes the conversion of glucose-1-phosphate to ADP-α-D-glucose. An exemplary glucose-1-phosphate adenylyltransferase (glgC) can be glgC from Escherichia coli (EC: 2.7.7.27, NCBI Accession WP_097472330.1). Also considered for downregulation or deletion use in the engineered cells provided herein are glgC homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID NO: 66 that have the activity of catalyzing the reversible conversion of glucose-1-phosphate to ADP-α-D-glucose.

Glucans biosynthesis glucosyltransferase (OpgG) is an enzyme involved in the conversion of UDP-glucose to MDO. An exemplary Glucans biosynthesis glucosyltransferase (OpgG) can be OpgG from Enterobacteriaceae (NCBI Accession WP_001343212.1) Also considered for use in the engineered cells provided herein are OpgG homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID NO: 67 that have the activity of catalyzing the reversible conversion of UDP-glucose to MDO.

Glucose-6-phosphate isomerase (pgi) catalyzes the reversible isomerization of glucose-6-phosphate to fructose-6-phosphate. An exemplary Glucose-6-phosphate isomerase (pgi) can be pgi from Escherichia coli (EC: 5.3.1.9, NCBI Accession AAC76995.1). Also considered for downregulation or deletion in the engineered cells provided herein are pgi homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID NO: 68 that have the activity of catalyzing the reversible conversion of glucose-6-phosphate to fructose-6-phosphate.

UDP-sugar hydrolase (ushA) is an enzyme with UDP-sugar hydrolase activities. An exemplary UDP-sugar hydrolase (ushA) can be ushA from Escherichia coli (EC: 3.1.3.5, NCBI Accession WP_000771748.1). Also considered for downregulation or deletion in the engineered cells provided herein are ushA homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID NO: 69 that have the activity of hydrolyzing UDP-sugars.

In addition to the modifications in the engineered cell lines discussed above, the invention further includes an engineered cell for producing UDP-galactose by overexpressing a nucleic acid sequence encoding UDP-glucose 4-epimerase (galE) activity, a nucleic acid sequence encoding galactokinase (galK) activity, and a nucleic acid sequence encoding galactose-1-phosphate uridylyltransferase (galT).

Galactokinase (galK) catalyzes the formation of galactose-6-phosphate from galactose. An exemplary galactokinase (galK) can be galK from Escherichia coli (EC: 2.7.1.6, NCBI Accession WP_000053415.1). Also considered for use in the engineered cells provided herein are galK homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID NO: 70 that have the activity of catalyzing the formation of galactose-6-phosphate from galactose.

Galactose-1-phosphate uridylyltransferase (galT) catalyzes the formation of UDP-galactose from galactose-1-phosphate. An exemplary galactose-1-phosphate uridylyltransferase (galT) can be galT from Escherichia coli (EC: 2.7.7.12, NCBI Accession WP_000191497.1). Also considered for use in the engineered cells provided herein are galT homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID NO: 71 that have the activity of catalyzing the formation of UDP-galactose from galactose-1-phosphate.

Fructokinase (cscK) phosphorylates fructose to generate fructose-6-phosphte. An exemplary fructokinase (cscK) can be cscK from Escherichia coli (EC 2.7.1.4, Accession WP 001274885.1). Also considered for use in the engineered cells provided herein are cscK homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID NO: 75 that have the activity of catalyzing the fructose-6-phosphate from fructose.

(heptosyl) lipopolysaccharide α-1,3-glucosyltransferase (WaaG) uses UDP-glucose as a substrate for lipopolysaccharide synthesis. An exemplary (heptosyl) lipopolysaccharide α-1,3-glucosyltransferase (WaaG) can be WaaG from Escherichia coli (EC 2.4. AAC76655.1). Also considered for use in the engineered cells provided herein are WaaG homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID NO: 76 that have the activity of catalyzing lipopolysaccharide synthesis from UDP-glucose.

Trehalose-6-phosphate synthase (otsA) catalyzes the biosynthesis of trehalose from UDP-glucose. An exemplary trehalose-6-phosphate synthase (otsA) can be otsA from Escherichia coli (EC 2.4.1.15, AAC74966.1). Also considered for use in the engineered cells provided herein are otsA homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID NO: 77 that have the activity of catalyzing lipopolysaccharide synthesis from UDP-glucose.

Glucose-1-phosphatase (agp) is a periplasmic protein that hydrolyses phosphate from glucose-1-phosphate and other substrates. An exemplary glucose-1-phosphatase (agp) can be agp from Escherichia coli (EC 3.1.3.10, Accession AAC74087.1). Also considered for use in the engineered cells provided herein are agp homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID NO: 78 that have the activity hydrolyzing phosphate from glucose-1-phosphate.

The invention further provides an engineered host cell that comprises one or more genetic modifications that result in enzymatic transformation by the engineered host cell of sucrose into uridine diphosphate glucose (UDP-glucose). The engineered host cell includes an exogenous nucleic acid sequence encoding sucrose transporter (cscB) activity or a homolog thereof, and exogenous expression nucleic acid sequence encoding sucrose synthase (SuSy) activity or a homolog thereof. The engineered host cell optionally further includes genetic modifications including, but not limited to, downregulation or deletion of UDP-glucose 4-epimerase (galE), downregulation or deletion of UDP-glucose 6-dehydrogenase (ugd), downregulation or deletion of glucans biosynthesis glucosyltransferase (mdoA), downregulation or deletion of glucose-1-phosphate adenylyltransferase (glgC), and downregulation or deletion of glucose-6-phosphate isomerase (pgi). In certain embodiments, the engineered cell comprises heterologous sucrose transporter (cscB) or a homolog thereof, heterologous sucrose synthase (SuSy) or a homolog thereof, and genetic modifications selected from a group consisting of: (i) downregulation or deletion of UDP-glucose 4-epimerase (galE) or a homolog thereof, (ii) downregulation or deletion of UDP-glucose 6-dehydrogenase (ugd) or a homolog thereof, (iii) downregulation or deletion of glucans biosynthesis glucosyltransferase (mdoA) or a homolog thereof, and (iv) any combinations thereof. In certain embodiments, the modifications listed herein may be combined with modifications listed in any other aspects of the invention. In certain embodiments, the modifications listed herein may be combined with one or modifications listed for the transformation of chemical intermediates into UDP-glucose.

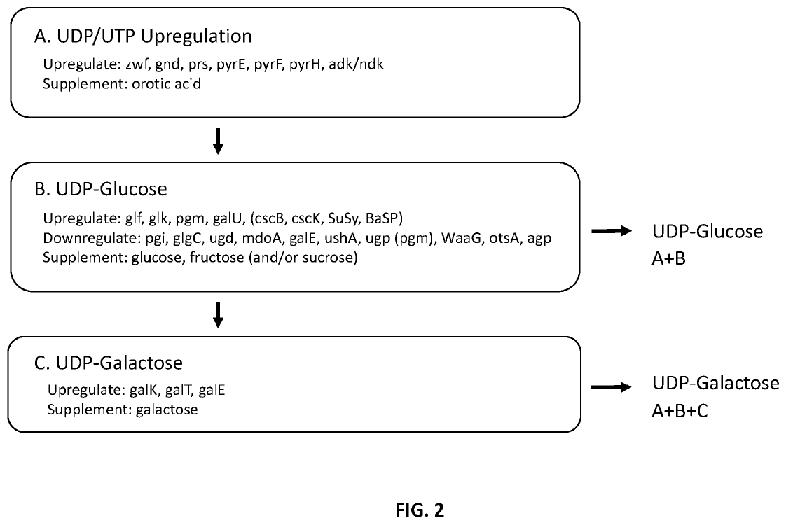

FIG. 2 of the invention provides an overview of the genetic modifications for the engineered host cell to increase the production of UDP-sugars, including UDP-glucose and UDP-galactose.

As described herein, the invention provides an engineered host cell that comprises one or more genetic modifications that result in enzymatic transformation by the engineered host cell of a carbon source through multiple chemical intermediates into a UDP-sugar. In certain embodiments, the UDP-sugar is glucose. In certain embodiments, the UDP-sugar is UDP-galactose. In certain embodiments, the chemical intermediate is UDP-glucose and the UDP-sugar is UDP-galactose. In certain embodiments, the engineered host cell is E. coli. In certain embodiments, the carbon source is selected from a group consisting of glycerol, glucose, sucrose, galactose, fructose, or any combination thereof.

As described in FIG. 2, the one or more genetic modifications in the engineered host cell result in an increased production of UDP-glucose. As described in FIG. 2, the one or more genetic modifications in the engineered host cell resulting in an enhanced production of UDP-glucose are selected from: overexpression of one or more genes selected from a group consisting of: glucose facilitator gene (glf) or a homolog thereof, glucokinase (glk) or a homolog thereof, phosphoglucomutase (pgm) or a homolog thereof, UTP-glucose-1-phosphate-uridylyltransferase (galU) or a homolog thereof, sucrose transporter (cscB) or a homolog thereof, sucrose synthase (SuSy) or a homolog thereof, sucrose phosphorylase (SPase) or a homolog thereof, glucose-6-phopshate 1-dehydrogenase (zwf) or a homolog thereof; 6-phosphogluconate dehydrogenase (gnd) or a homolog thereof; phosphoribosylpyrophosphate synthetase (prs) or a homolog thereof; phosphoribosyltransferase (pyrE) or a homolog thereof; orotidine 5′-phosphate decarboxylase (pyrF) or a homolog thereof, uridylate kinase (pyrH) or a homolog thereof, nucleoside diphosphate kinase (ndk) or a homolog thereof, and adenylate kinase (adk) or a homolog thereof; and/or downregulation or deletion of one or more genes selected from a group consisting of: glucose-6-phosphate isomerase (pgi), glucose-1-phosphate adenylyltransferase (glgC), UDP-glucose 6-dehydrogenase (ugd), glucans biosynthesis glucosyltransferase (OpgG), UDP-glucose 4-epimerase (galE), UDP-sugar hydrolase (ushA), UTP-glucose-1-phosphate uridylyltransferase (ugp), phosphoglucomutase (pgm); and/or any combinations thereof. As described in FIG. 2, the engineered host cells are supplemented with a medium comprising: orotic acid, sucrose, glucose, or a combination thereof.

As described in FIG. 2, the one or more genetic modifications in the engineered host cell result in an increased production of UDP-galactose. As described in FIG. 2, the one or more genetic modifications in the engineered host cell resulting in an enhanced production of UDP-galactose are selected from: (i) overexpression of UDP-glucose 4-epimerase (galE) or a homolog thereof, (ii) overexpression of galactokinase (galK) or a homolog thereof, (iii) overexpression of galactose-1-phosphate uridylyltransferase (galT) or a homolog thereof, and (iv) any combinations thereof. As described in FIG. 2, the engineered host cells are supplemented with a medium comprising galactose.

As described in FIG. 2, the one or more genetic modifications in the engineered host cell for increasing the production of UDP-galactose include one or more genetic modifications resulting in an increased production of UDP-glucose. Accordingly, as described in FIG. 2, the one or more genetic modifications to increase the production of UDP-galactose are selected from: overexpression of one or more genes selected from a group consisting of: glucose facilitator gene (glf) or a homolog thereof, glucokinase (glk) or a homolog thereof, phosphoglucomutase (pgm) or a homolog thereof, UTP-glucose-1-phosphate-uridylyltransferase (galU) or a homolog thereof, sucrose transporter (cscB) or a homolog thereof, sucrose synthase (SuSy) or a homolog thereof, sucrose phosphorylase (SPase) or a homolog thereof, glucose-6-phopshate 1-dehydrogenase (zwf) or a homolog thereof; 6-phosphogluconate dehydrogenase (gnd) or a homolog thereof; phosphoribosylpyrophosphate synthetase (prs) or a homolog thereof; phosphoribosyltransferase (pyrE) or a homolog thereof; orotidine 5′-phosphate decarboxylase (pyrF) or a homolog thereof, uridylate kinase (pyrH) or a homolog thereof, nucleoside diphosphate kinase (ndk) or a homolog thereof, and adenylate kinase (adk) or a homolog thereof; overexpression of UDP-glucose 4-epimerase (galE) or a homolog thereof, overexpression of galactokinase (galK) or a homolog thereof, overexpression of galactose-1-phosphate uridylyltransferase (galT) or a homolog thereof; and/or downregulation or deletion of one or more genes selected from a group consisting of: glucose-6-phosphate isomerase (pgi), glucose-1-phosphate adenylyltransferase (glgC), UDP-glucose 6-dehydrogenase (ugd), glucans biosynthesis glucosyltransferase (OpgG), UDP-sugar hydrolase (ushA), UTP-glucose-1-phosphate uridylyltransferase (ugp), phosphoglucomutase (pgm); and/or any combinations thereof. As described in FIG. 2, the engineered host cells are supplemented with a medium comprising: glucose, galactose, orotic acid, sucrose, or any combination thereof.

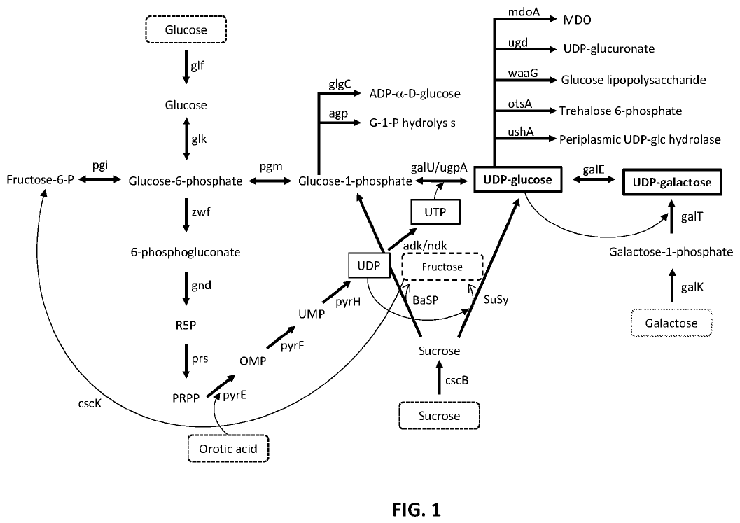

FIG. 1 herein provides an exemplary embodiment illustrating a combination of modifications to the E. coli host genome including deletions and overexpression of enzymes from other organisms to recapitulate the bioproduction pathway described in that figure.

The invention provides an engineered host cell that comprises one or more genetic modifications (as shown in FIG. 1 and described in this Example 1 and herein above in this application) that result in enzymatic transformation by the engineered host cell of glucose through multiple chemical intermediates into UDP-glucose. As shown in FIG. 1, in certain embodiments, one or more of the genetic modifications lead to increased metabolic flux to UDP-glucose precursors. As shown in FIG. 1, in certain embodiments, one or more of the genetic modifications cause reduction of formation of byproducts. As shown in FIG. 1, in certain embodiments, the genetic modification is selected from a group consisting of (i) glucose facilitator gene (glf) or a homolog thereof, (ii) glucokinase (glk) or a homolog thereof, (iii) phosphoglucomutase (pgm) or a homolog thereof, (iv) UTP-glucose-1-phosphate-uridylyltransferase (galU) or a homolog thereof, (v) phosphoribosyltransferase (pyrE) or a homolog thereof, (vi) expression of orotidine 5′-phosphate decarboxylase (pyrF) or a homolog thereof, (vii) uridylate kinase (pyrH) or a homolog thereof, (viii) nucleoside diphosphate kinase (ndk) or a homolog thereof, (ix) adenylate kinase (adk) or a homolog thereof; and (x) any combinations thereof. In certain embodiments, the one or more genetic modification is overexpression of fructokinase (cscK) or a homolog thereof.

As shown in FIG. 1, in certain embodiments, the one or more genetic modifications is overexpression of one or more genes selected from a group consisting of: (i) glucose facilitator gene (glf) or a homolog thereof, (ii) glucokinase (glk) or a homolog thereof, (iii) phosphoglucomutase (pgm) or a homolog thereof, (iv) UTP-glucose-1-phosphate-uridylyltransferase (galU) or a homolog thereof, (v) glucose-6-phopshate 1-dehydrogenase (zwf) or a homolog thereof, (vi) 6-phosphogluconate dehydrogenase (gnd) or a homolog thereof, (vii) phosphoribosylpyrophosphate synthetase (prs) or a homolog thereof; and (viii) any combinations thereof.

As shown in FIG. 1, in certain embodiments, the one or more genetic modifications is selected from a group consisting of: (i) glucose facilitator gene (glf) or a homolog thereof, (ii) glucokinase (glk) or a homolog thereof; and (iii) any combinations thereof. In certain embodiments, the one or more genetic modifications is overexpression of phosphoglucomutase (pgm) or a homolog thereof. In certain embodiments, the one or more genetic modifications is overexpression of UTP-glucose-1-phosphate-uridylyltransferase (galU) or a homolog thereof.

As shown in FIG. 1, in certain embodiments, one or more genetic modifications is overexpression of orotate phosphoribosyltransferase (pyrE) or a homolog thereof. In certain embodiments, one or more genetic modifications is overexpression of orotidine 5′-phosphate decarboxylase (pyrF) or a homolog thereof. In certain embodiments, a medium comprising orotic acid is also provided. In certain embodiments, the one or more genetic modifications is overexpression of uridylate kinase (pyrH) or a homolog thereof.

As shown in FIG. 1, in certain embodiments, the one or more genetic modifications is overexpression of nucleoside diphosphate kinase (ndk) or a homolog thereof. In certain embodiments, the one or more genetic modifications is overexpression of adenylate kinase (adk) or a homolog thereof.

As shown in FIG. 1, in certain embodiments, the one or more genetic modifications is downregulation or deletion of UDP-glucose 4-epimerase (galE). As shown in FIG. 1, in certain embodiments, the one or more genetic modifications is downregulation or deletion of UDP-glucose 6-dehydrogenase (ugd) to prevent conversion of UDP-glucose to UDP-glucuronate.

As shown in FIG. 1, in certain embodiments, the one or more genetic modifications is downregulation or deletion of glucose-1-phosphate adenylyltransferase (glgC).

As shown in FIG. 1, in certain embodiments, the one or more genetic modifications is downregulation or deletion of glucans biosynthesis glucosyltransferase (OpgG) to prevent conversion of UDP-glucose to membrane-derived oligosaccharides (MDO).

As shown in FIG. 1, in certain embodiments, the one or more genetic modifications is downregulation or deletion of glucose-6-phosphate isomerase (pgi).

As shown in FIG. 1, in certain embodiments, the one or more genetic modifications is overexpression of glucose-6-phopshate 1-dehydrogenase (zwf) or a homolog thereof.

As shown in FIG. 1, in certain embodiments, the one or more genetic modifications is overexpression of 6-phosphogluconate dehydrogenase (gnd) or a homolog thereof.

As shown in FIG. 1, in certain embodiments, the one or more genetic modifications is overexpression of phosphoribosylpyrophosphate synthetase (prs) or a homolog thereof.

As shown in FIG. 1, in certain embodiments, wherein the engineered host cells include overexpression of: (i) glucose facilitator gene (glf) or a homolog thereof, (ii) glucokinase (glk) or a homolog thereof, (iii) phosphoglucomutase (pgm) or a homolog thereof, (iv) UTP-glucose-1-phosphate-uridylyltransferase (galU) or a homolog thereof, (v) phosphoribosyltransferase (pyrE) or a homolog thereof, (vi) orotidine 5′-phosphate decarboxylase (pyrF) or a homolog thereof. (vii) uridylate kinase (pyrH) or a homolog thereof, (viii) nucleoside diphosphate kinase (ndk) or a homolog thereof, and (ix) adenylate kinase (adk) or a homolog thereof.

As shown in FIG. 1, in certain embodiments, the engineered host cells include overexpression of: (i) glucose facilitator gene (glf) or a homolog thereof, (ii) glucokinase (glk) or a homolog thereof, (iii) phosphoglucomutase (pgm) or a homolog thereof, (iv) UTP-glucose-1-phosphate-uridylyltransferase (galU) or a homolog thereof, (v) glucose-6-phopshate 1-dehydrogenase (zwf) or a homolog thereof, (vi) 6-phosphogluconate dehydrogenase (gnd) or a homolog thereof, and (vii) phosphoribosylpyrophosphate synthetase (prs) or a homolog thereof.

Aspects of the invention are now described with reference to FIG. 1.

Step 1: conversion of glucose to glucose-6-phosphate, glucose facilitator gene (glf) or a homolog thereof, and glucokinase (glk) or a homolog thereof are overexpressed. In addition, phosphotransferase enzyme or a homolog thereof may be overexpressed.

Step 2: conversion of glucose-6-phosphate to glucose-1-phosphate, phosphoglucomutase (pgm) or a homolog thereof are overexpressed.

Step 3: conversion of glucose-1-phosphate and UTP to UDP-glucose. UTP-glucose-1-phosphate-uridylyltransferase (galU) or a homolog thereof are overexpressed.

Step 4: conversion of 5-phosphoribosyl 1-pyrophosphate (PRPP) to uridine-monophosphate (UMP). Orotate phosphoribosyltransferase (pyrE) or a homolog thereof and orotidine 5′-phosphate decarboxylase (pyrF) or a homolog thereof are overexpressed. In certain embodiments, this step includes supplementation with orotic acid.

Step 5: conversion of uridine-monophosphate (UMP) to uridine-diphosphate (UDP). Uridylate kinase (pyrH) or a homolog thereof is overexpressed.

Step 6: conversion of uridine-diphosphate (UDP) to uridine-triphosphate (UTP). Nucleoside diphosphate kinase (ndk) or a homolog thereof and/or adenylate kinase (adk) or a homolog thereof are overexpressed.

Step 7: conversion of UDP-glucose to UDP-galactose. UDP-glucose 4-epimerase (galE) is downregulated or deleted to prevent generation of UDP-galactose.

Step 8: conversion of UDP-glucose to UDP-glucuronate. UDP-glucose 6-dehydrogenase (ugd) is downregulated or deleted to prevent conversion of UDP-glucose to UDP-glucuronate.

Step 9: conversion of glucose-1-phosphate to ADP-α-D-glucose, glucose-1-phosphate adenylyltransferase (glgC) is downregulated or deleted to prevent conversion of UDP-glucose to ADP-α-D-glucose.

Step 10: conversion of UDP-glucose to membrane-derived oligosaccharides (MDO). glucans biosynthesis glucosyltransferase (OpgG) is downregulated or deleted to prevent conversion of UDP-glucose to of membrane-derived oligosaccharides (MDO), including to ADP-α-D-glucose.

Step 11: conversion of glucose-6-phosphate to fructose-6-phosphate. Glucose-6-phosphate isomerase (pgi) is downregulated or deleted to prevent the glucose from being metabolized through the glycolysis pathway.

Step 12: conversion of glucose-6-phosphate to 6-phosphogluconate. Glucose-6-phopshate 1-dehydrogenase (zwf) or a homolog thereof may be overexpressed.

Step 13: conversion of 6-phosphogluconate to ribose-5-phosphate (R5P). 6-phosphogluconate dehydrogenase (gnd) or a homolog thereof may be overexpressed.

Step 14: conversion of ribose-5-phosphate (R5P) to 5-phosphoribosyl 1-pyrophosphate (PRPP). Phosphoribosylpyrophosphate synthetase (prs) or a homolog thereof may be overexpressed.

In certain preferred embodiments, the engineered host cells comprise one or more genetic modifications recited above in steps 1-3 and 4-6. In these preferred embodiments, the engineered host cells may include overexpression of: (i) glucose facilitator gene (glf) or a homolog thereof, (ii) glucokinase (glk) or a homolog thereof, (iii) phosphoglucomutase (pgm) or a homolog thereof, (iv) UTP-glucose-1-phosphate-uridylyltransferase (galU) or a homolog thereof, (v) phosphoribosyltransferase (pyrE) or a homolog thereof, (vi) orotidine 5′-phosphate decarboxylase (pyrF) or a homolog thereof, (vii) uridylate kinase (pyrH) or a homolog thereof, (viii) nucleoside diphosphate kinase (ndk) or a homolog thereof, and (ix) adenylate kinase (adk) or a homolog thereof.

In certain preferred embodiments, the engineered cell lines comprise one or more genetic modifications recited above in steps 1-3 and 12-14. In these preferred embodiments, the engineered host cells comprise overexpression of: (i) glucose facilitator gene (glf) or a homolog thereof, (ii) glucokinase (glk) or a homolog thereof, (iii) phosphoglucomutase (pgm) or a homolog thereof, (iv) UTP-glucose-1-phosphate-uridylyltransferase (galU) or a homolog thereof, (v) glucose-6-phopshate 1-dehydrogenase (zwf) or a homolog thereof, (vi) 6-phosphogluconate dehydrogenase (gnd) or a homolog thereof, and (vii) phosphoribosylpyrophosphate synthetase (prs) or a homolog thereof.

In certain preferred embodiments, the engineered cell lines comprise one or more genetic modifications recited above in steps 1-3, 4-6, and 12-14. In these preferred embodiments, the engineered host cells may include overexpression of: (i) glucose facilitator gene (glf) or a homolog thereof, (ii) glucokinase (glk) or a homolog thereof, (iii) phosphoglucomutase (pgm) or a homolog thereof, (iv) UTP-glucose-1-phosphate-uridylyltransferase (galU) or a homolog thereof, (v) phosphoribosyltransferase (pyrE) or a homolog thereof, (vi) orotidine 5′-phosphate decarboxylase (pyrF) or a homolog thereof, (vii) uridylate kinase (pyrH) or a homolog thereof, (viii) nucleoside diphosphate kinase (ndk) or a homolog thereof, (ix) adenylate kinase (adk) or a homolog thereof, (x) glucose-6-phopshate 1-dehydrogenase (zwf) or a homolog thereof, (xi) 6-phosphogluconate dehydrogenase (gnd) or a homolog thereof, and (xii) phosphoribosylpyrophosphate synthetase (prs) or a homolog thereof.

In another aspect, the invention provides a method of increasing the production of uridine diphosphate glucose (UDP-glucose) by utilizing the engineered host cells described in this example.

In another aspect, the invention provides an engineered host cell that comprises one or more genetic modifications (as shown in FIG. 1 and described and herein above in this application) that result in enzymatic transformation by the engineered host cell of glucose and/or galactose through multiple chemical intermediates into UDP-galactose.

As shown in FIG. 1, in another aspect, the invention provides an engineered host cell that comprises one or more genetic modifications (as shown in FIG. 1 and described in this Example 1 and herein above in this application) that result in enzymatic transformation of glucose by the engineered host cell of glucose and/or galactose through multiple intermediates into UDP-galactose. As shown in FIG. 1, in certain embodiments, in addition to all the genetic modifications listed above, one or more of the genetic modifications lead to increased metabolic flux to UDP-galactose precursors. As shown in FIG. 1, in certain embodiments, one or more of the genetic modifications cause reduction of formation of byproducts.

As shown in FIG. 1, in certain embodiments, the one or more genetic modification is selected from a group consisting of: (i) overexpression of UDP-glucose 4-epimerase (galE) or a homolog thereof, (ii) overexpression of galactokinase (galK) or a homolog thereof, (iii) overexpression of galactose-1-phosphate uridylyltransferase (galT) or a homolog thereof, and (iv) any combinations thereof.

As shown in FIG. 1, in certain embodiments, the one or more genetic modifications is overexpression of UDP-glucose 4-epimerase (galE) or a homolog thereof.

As shown in FIG. 1, in certain embodiments, the one or more genetic modifications is overexpression of galactokinase (galK) or a homolog thereof. In certain embodiments, a supplement comprising galactose is also provided.

As shown in FIG. 1, in certain embodiments, the one or more genetic modifications is overexpression of galactose-1-phosphate uridylyltransferase (galT) or a homolog thereof.

As shown in FIG. 1, in another aspect, the invention provides an engineered host cell that comprises one or more genetic modifications (as shown in FIG. 1 and described in this Example 1 and herein above in this application) that result in enzymatic transformation by the engineered host cell of glucose through multiple intermediates into UDP-glucose.

Aspects of the invention are now described with reference to FIG. 1.

Step 1: conversion of glucose to glucose-6-phosphate. glucose facilitator gene (glf) or a homolog thereof and/or glucokinase (glk) or a homolog thereof is overexpressed. In addition, phosphotransferase enzyme or a homolog thereof may be overexpressed.

Step 2: conversion of glucose-6-phosphate to glucose-1-phosphate. phosphoglucomutase (pgm) or a homolog thereof are overexpressed.

Step 3: conversion of glucose-1-phosphate and UTP to UDP-glucose. UTP-glucose-1-phosphate-uridylyltransferase (galU) or a homolog thereof are overexpressed.

Step 4: conversion of 5-phosphoribosyl 1-pyrophosphate (PRPP) to uridine-monophosphate (UMP). Orotate phosphoribosyltransferase (pyrE) or a homolog thereof and/or orotidine 5′-phosphate decarboxylase (pyrF) or a homolog thereof is overexpressed. In certain embodiments, a supplement comprising orotic acid is also provided.

Step 5: conversion of uridine-monophosphate (UMP) to uridine-diphosphate (UDP). Uridylate kinase (pyrH) or a homolog thereof is overexpressed.

Step 6: conversion of uridine-diphosphate (UDP) to uridine-triphosphate (UTP). Nucleoside diphosphate kinase (ndk) or a homolog thereof and/or adenylate kinase (adk) or a homolog thereof are overexpressed.

Step 7: conversion of UDP-glucose to UDP-galactose. UDP-glucose 4-epimerase (galE) or a homolog thereof is overexpressed.

Step 8: conversion of UDP-glucose to UDP-glucuronate. UDP-glucose 6-dehydrogenase (ugd) is downregulated or deleted to prevent generation of UDP-glucuronate.

Step 9: conversion of glucose-1-phosphate to ADP-α-D-glucose. Glucose-1-phosphate adenylyltransferase (glgC) is downregulated or deleted to prevent the generation of UDP-glucuronate.

Step 10: conversion of UDP-glucose to membrane-derived oligosaccharides (MDO). Glucans biosynthesis glucosyltransferase (OpgG) is downregulated or deleted to prevent formation of membrane-derived oligosaccharides (MDO), including to ADP-α-D-glucose.

Step 11: conversion of glucose-6-phosphate to fructose-6-phosphate. Glucose-6-phosphate isomerase (pgi) is downregulated or deleted to prevent the glucose being metabolized through the glycolysis pathway.

Step 12: conversion of glucose-6-phosphate to 6-phosphogluconate. Glucose-6-phopshate 1-dehydrogenase (zwf) or a homolog thereof may be overexpressed.

Step 13: conversion of 6-phosphogluconate to ribose-5-phosphate (R5P). 6-phosphogluconate dehydrogenase (gnd) or a homolog thereof may be overexpressed.

Step 14: conversion of ribose-5-phosphate (R5P) to 5-phosphoribosyl 1-pyrophosphate (PRPP). Phosphoribosylpyrophosphate synthetase (prs) or a homolog thereof may be overexpressed.

Step 15: conversion of galactose to galactose-1-phosphate. Galactokinase (galK) or a homolog thereof is overexpressed. In certain embodiments, a supplement comprising galactose was also provided.

Step 16: conversion of galactose-1-phosphate to UDP-galactose. galactose-1-phosphate uridylyltransferase (galT) or a homolog thereof is overexpressed.

In certain preferred embodiments, the engineered host cells comprise one or more genetic modifications recited above in steps 1-3, 4-7, and 15-16. In these preferred embodiments, the engineered host cells may include overexpression of: (i) glucose facilitator gene (glf) or a homolog thereof, (ii) glucokinase (glk) or a homolog thereof, (iii) phosphoglucomutase (pgm) or a homolog thereof, (iv) UTP-glucose-1-phosphate-uridylyltransferase (galU) or a homolog thereof, (v) Orotate phosphoribosyltransferase (pyrE) or a homolog thereof, (vi) orotidine 5′-phosphate decarboxylase (pyrF) or a homolog thereof is overexpressed, (vii) uridylate kinase (pyrH) or a homolog thereof, (viii) nucleoside diphosphate kinase (ndk) or a homolog thereof, (ix) adenylate kinase (adk) or a homolog thereof, (x) overexpression of UDP-glucose 4-epimerase (galE) or a homolog thereof, (xi) overexpression of galactokinase (galK) or a homolog thereof, and (xii) overexpression of galactose-1-phosphate uridylyltransferase (galT) or a homolog thereof.

In certain preferred embodiments, the engineered cell lines comprise one or more genetic modifications recited above in steps 1-3, 7, and 12-16. In these preferred embodiments, the engineered host cells comprise overexpression of: (i) glucose facilitator gene (glf) or a homolog thereof, (ii) glucokinase (glk) or a homolog thereof, (iii) phosphoglucomutase (pgm) or a homolog thereof, (iv) UTP-glucose-1-phosphate-uridylyltransferase (galU) or a homolog thereof, (v) glucose-6-phopshate 1-dehydrogenase (zwf) or a homolog thereof, (vi) 6-phosphogluconate dehydrogenase (gnd) or a homolog thereof, (vii) phosphoribosylpyrophosphate synthetase (prs) or a homolog thereof, (viii) overexpression of UDP-glucose 4-epimerase (galE) or a homolog thereof, (ix) overexpression of galactokinase (galK) or a homolog thereof, and (x) overexpression of galactose-1-phosphate uridylyltransferase (galT) or a homolog thereof. In certain embodiments, a supplement comprising galactose is also provided.

In certain preferred embodiments, the engineered cell lines comprise one or more genetic modifications recited above in steps 7, 15, and 16. In these preferred embodiments, the engineered host cells comprise overexpression of: (i) UDP-glucose 4-epimerase (galE or a homolog thereof), (ii) galactokinase (galK) or a homolog thereof, and (iii) galactose-1-phosphate uridylyltransferase (galT) or a homolog thereof. In certain embodiments, a supplement comprising galactose is also provided.

In another aspect, the invention provides a method of increasing the production uridine diphosphate galactose (UDP-galactose) by utilizing the engineered host cells described in this example.

In another aspect, the invention provides an engineered host cell that comprises one or more genetic modifications (as shown in FIG. 1 and described in herein above in this application) that result in enzymatic transformation by the engineered host cell of sucrose through multiple chemical intermediates into UDP-glucose.

In certain embodiments, the genetic modifications in the engineered host cells are selected from a group consisting of: (i) heterologous expression of sucrose transporter (cscB) or a homolog thereof, (ii) heterologous expression of sucrose synthase (SuSy) or a homolog thereof, (iii) downregulation or deletion of UDP-glucose 4-epimerase (galE), (iv) downregulation or deletion of UDP-glucose 6-dehydrogenase (ugd), (v) downregulation or deletion of glucans biosynthesis glucosyltransferase (OpgG), and (vi) any combinations thereof.

In certain embodiments, the engineered host cell comprises heterologous sucrose transporter (cscB), heterologous sucrose synthase (SuSy), and genetic modifications selected from a group consisting of: (i) downregulation or deletion of UDP-glucose 4-epimerase (galE), (ii) downregulation or deletion of UDP-glucose 6-dehydrogenase (ugd), (iii) downregulation or deletion of glucans biosynthesis glucosyltransferase (OpgG), and (iv) any combinations thereof.

As shown in FIG. 1, in another aspect, the invention provides an engineered host cell that comprises one or more genetic modifications (as shown in FIG. 1 and described in this Example 1 and herein above in this application) that result in enzymatic transformation by the engineered host cell of sucrose into UDP-glucose. Moreover, as described in FIG. 1, the invention provides that the engineered host cell results in enzymatic transformation of sucrose to UDP-glucose via sucrose phosphorylase (SPase) or a homolog thereof and/or sucrose synthase (SuSy) or a homolog thereof. Accordingly, the invention provides for overexpression of sucrose phosphorylase (SPase) or a homolog thereof and/or sucrose synthase (SuSy) or a homolog thereof to increase the transformation of sucrose to UDP-glucose.

Aspects of the invention are now described with reference to FIG. 1.

Step 1: conversion of glucose to glucose-6-phosphate. glucose facilitator gene (glf) or a homolog thereof and/or glucokinase gene (glk) or a homolog thereof are overexpressed. In addition, phophotransferase enzyme or a homolog thereof may be overexpressed.

Step 2: conversion of glucose-6-phosphate to glucose-1-phosphate. phosphoglucomutase (pgm) or a homolog thereof are overexpressed.

Step 3: conversion of glucose-1-phosphate and UTP to UDP-glucose. UTP-glucose-1-phosphate-uridylyltransferase (galU) or a homolog thereof are overexpressed.

Step 4: conversion of 5-phosphoribosyl 1-pyrophosphate (PRPP) to uridine-monophosphate (UMP). Orotate phosphoribosyltransferase (pyrE) or a homolog thereof and/or orotidine 5′-phosphate decarboxylase (pyrF) is overexpressed. In certain embodiments, a supplement comprising orotic acid is also provided.

Step 5: conversion of uridine-monophosphate (UMP) to uridine-diphosphate (UDP). Uridylate kinase (pyrH) or a homolog thereof are overexpressed.

Step 6: conversion of uridine-diphosphate (UDP) to uridine-triphosphate (UTP). Nucleoside diphosphate kinase (ndk) or a homolog thereof and/or adenylate kinase (adk) or a homolog thereof are overexpressed.

Step 7: conversion of UDP-glucose to UDP-galactose. UDP-glucose 4-epimerase (GalE) is downregulated or deleted to prevent conversion of UDP-glucose to UDP-galactose.

Step 8: conversion of UDP-glucose to UDP-glucuronate. UDP-glucose 6-dehydrogenase (ugd) is downregulated or deleted to prevent the conversion of UDP-glucose to UDP-glucuronate.

Step 9: conversion of glucose-1-phosphate to ADP-α-D-glucose, glucose-1-phosphate adenylyltransferase (glgC) is downregulated or deleted to prevent the conversation of UDP-glucose to UDP-glucuronate.

Step 10: conversion of UDP-glucose to membrane-derived oligosaccharides (MDO), glucans biosynthesis glucosyltransferase (OpgG) is downregulated or deleted to prevent the conversion of UDP-glucose to membrane-derived oligosaccharides (MDO), including to ADP-α-D-glucose.

Step 11: conversion of glucose-6-phosphate to fructose-6-phosphate. Glucose-6-phosphate isomerase (pgi) is downregulated or deleted to prevent the glucose being metabolized through the glycolysis pathway.

Step 12: conversion of glucose-6-phosphate to 6-phosphogluconate. Glucose-6-phopshate 1-dehydrogenase (zwf) or a homolog thereof may be overexpressed.

Step 13: conversion of 6-phosphogluconate to ribose-5-phosphate (R5P). 6-phosphogluconate dehydrogenase (gnd) or a homolog thereof may be overexpressed.

Step 14: conversion of ribose-5-phosphate (R5P) to 5-phosphoribosyl 1-pyrophosphate (PRPP). Phosphoribosylpyrophosphate synthetase (prs) or a homolog thereof may be overexpressed.

In another aspect, the invention provides a method of increasing the production of uridine diphosphate glucose (UDP-glucose) by utilizing the engineered host cells described in this example.

Table 1 below provides a list of the sequences of exemplary enzymes of the invention.

TABLE 1
Exemplary Enzyme Sequences of the Invention

| SEQ ID | | | |
|---|---|---|---|
| NO | Gene | Accession | Sequence |
| 1 | glf | AAA27691.1 | MSSESSQGLVTRLALIAAIGGLLFGYDSAVIAAIGTPVDIHFIAP |
| | | | RHLSATAAASLSGMVVVAVLVGCVTGSLLSGWIGIRFGRRGG |
| | | | LLMSSICFVAAGFGAALTEKLFGTGGSALQIFCFFRFLAGLGIG |
| | | | VVSTLTPTYIAEIRPPDKRGQMVSGQQMAIVTGALTGYIFTWL |
| | | | LAHFGSIDWVNASGWCWSPASEGLIGIAFLLLLLTAPDTPHW |
| | | | LVMKGRHSEASKILARLEPQADPNLTIQKIKAGFDKAMDKSS |
| | | | AGLFAFGITVVFAGVSVAAFQQLVGINAVLYYAPQMFQNLGF |
| | | | GADTALLQTISIGVVNFIFTMIASRVVDRFGRKPLLIWGALGM |
| | | | AAMMAVLGCCFWFKVGGVLPLASVLLYIAVFGMSWGPVCW |
| | | | VVLSEMFPSSIKGAAMPIAVTGQWLANILVNFLFKVADGSPA |
| | | | LNQTFNHGFSYLVFAALSILGGLIVARFVPETKGRSLDEIEEM |
| | | | WRSQK |
| 2 | glk | AAA27694.1 | MEIVAIDIGGTHARFSIAEVSNGRVLSLGEETTFKTAEHASLQL |
| | | | AWERFGEKLGRPLPRAAAIAWAGPVHGEVLKLTNNPWVLRP |
| | | | ATLNEKLDIDTHVLINDFGAVAHAVAHMDSSYLDHICGPDEA |
| | | | LPSDGVITILGPGTGLGVAHLLRTEGRYFVIETEGGHIDFAPLD |
| | | | RLEDKILARLRERFRRVSIERIISGPGLGNIYEALAAIEGVPFSL |
| | | | LDDIKLWQMALEGKDNLAEAALDRFCLSLGAIAGDLALAQG |
| | | | RTSVVIGGGVGLRIASHLPESGFRQRFVSKGRFERVMSKIPVK |
| | | | LITYPQPGLLGAQLPMPTNILKLNNIF |
| 3 | pgm | AAC73782.1 | MAIHNRAGQPAQQSDLINVAQLTAQYYVLKPEAGNAEHAVK |
| | | | FGTSGHRGSAARHSFNEPHILAIAQAIAEERAKNGITGPCYVG |
| | | | KDTHALSEPAFISVLEVLAANGVDVIVQENNGFTPTPAVSNAI |
| | | | LVHNKKGGPLADGIVITPSHNPPEDGGIKYNPPNGGPADTNVT |
| | | | KVVEDRANALLADGLKGVKRISLDEAMASGHVKEQDLVQPF |
| | | | VEGLADIVDMAAIQKAGLTLGVDPLGGSGIEYWKRIGEYYNL |
| | | | NLTIVNDQVDQTFRFMHLDKDGAIRMDCSSECAMAGLLALR |
| | | | DKFDLAFANDPDYDRHGIVTPAGLMNPNHYLAVAINYLFQH |
| | | | RPQWGKDVAVGKTLVSSAMIDRVVNDLGRKLVEVPVGFKW |
| | | | FVDGLFDGSFGFGGEESAGASFLRFDGTPWSTDKDGIIMCLLA |
| | | | AEITAVTGKNPQEHYNELAKRFGAPSYNRLQAAATSAQKAA |
| | | | LSKLSPEMVSASTLAGDPITARLTAAPGNGASIGGLKVMTDN |
| | | | GWFAARPSGTEDAYKIYCESFLGEEHRKQIEKEAVEIVSEVLK |
| | | | NA |
| 4 | pgm | A0A1F1X9E9 | MAHERAGQLAQPSDLIDIAELVTAYYTRTPDADNPDQQVAFG |
| | | | TSGHRGSALDTAFNEAHILAITQAIVDYRAEQNTTGAIFIGRDT |
| | | | HALSEPAMVSALEVLLANEVEVRVDDRGRYTPTPAVSHAILT |
| | | | NPGTDGIVITPSHNPPRDGGFKYNPPTGGPADTDATDWIAAK |
| | | | ANEYLRAGLEGVKRVPVSGVLDERCVKHNYLDTYVADLEN |
| | | | VVDMKAIKEAGVRIGADPMGGASVDYWAAIAEHYGLAMTV |
| | | | VNPEVDGTFRFMTLDTDGKIRMDCSSPNAMASLVDNRAKYD |
| | | | LATGNDADADRHGIVTPDAGLMNPNHYLAVAIEYLFSHRPQ |
| | | | WGNAGVGKTLVSSSMIDRVVANLGRELVEVPVGFKWFVPGL |
| | | | VDGSLGFGGEESAGASFLRFDGSVWSTDKDGIILDLLAAEILA |
| | | | VTGKTPSQRYAELAEEYGAPAYARTDAPANREQKAILKKLSP |
| | | | EQVTAMELAGEEILAKLTEAPGNGAAIGGLKVTTENAWFAA |
| | | | RPSGTEDKYKIYAESFRGEEHLKQVQEEAQALVSQVLGS |
| 5 | pgm | A0A1I0EI47 | MAKHPRAGQQPTSCDLINVAKLTSQYYVLQPALHESAHAVK |
| | | | FGTSGHRGSSTKHTFNEAHILAIAQAIAEIRHAKGITGPCYVGK |
| | | | DTHALSEPAFISVLEVLAANQVTVIIQPENGFTPTPAISHAILTH |
| | | | NKSNPIHLADGIVITPSHNPPEDGGIKYNPPNGGPADTDFTGLI |
| | | | EKRANQLLDERSSENAVLAGVKRISLSQAISSQFIIQQDMMMP |
| | | | YVEDLVNVVDLSVIQQAGLKLGVDPLGGSGIAYWQQIAEYY |
| | | | KLNLSLVNDQVDQTFRFMPLDHDGVIRMDCSSVDAMGGLLR |
| | | | LKDKFDLAFANDPDYDRHGIVTPAGLMNPNHYLSVAINYLY |
| | | | QNRPGWPVDLGVGKTLVSSAMIDRVVSSLNRKLVEVPVGFK |
| | | | WFVDGLYTGKLGFGGEESAGASFLRFDAKPWSTDKDGIIMCL |
| | | | LSAEITARTGKNPQVHYDELSEKFGAPIYNRIQANATHEQKAI |
| | | | LSKLSPDQVQAKTLAGDLITARITHAPSNNASIGGLKVVTDNG |
| | | | WFAARPSGTEEAYKIYCESFISHEHLDLISKEAVEIVDAAFASN |
| | | | K |
| 6 | pgm | A0A1M5 | MAIHPRAGQKAQQGDLNNIPALMSDYYRLQPDPSNKNHQVE |
| | | W315 | FGTSGHRGSSCKCTFNETHILAITQAIVEVRAARGIEGPLFVGK |
| | | | DTHALSESAFSSVVEVLIANHVGVIAQEGKSYTPTPGISHAILT |
| | | | FNRTYPDVLSDGIVITPSHNPPQDGGIKYNPPHGGPAEAEITQA |
| | | | IESRANEIIANQLKAVKRVPYESAVSSVLYVEQDLVAPYVDDL |
| | | | VNVIDMKAIQKANLKIGIDPLGGSGIEYWRQIAATYELNLHLV |
| | | | NEAVDPTFQFMTLDKDGVIRMDCSSPYAMAGLLALKDEYDL |
| | | | AFGNDPDYDRHGIVTPKGLMNPNHFLAVCIDYLFRHRQWDQ |
| | | | SVAVGKTLVSSALIDRVVGDLGRELCEVPVGFKWFVEGLFHG |
| | | | KFGFAGEESAGASLLRFDGTPWATDKDGIILCLLAAEITAVTG |
| | | | KNPQQYYEALAAKHGESLYSRIQAVASASQKDVLKKMSPEM |
| | | | VSAETLAGDPITARLTHAPGNGAAIGGLKVTTDFGWFAARPS |
| | | | GTEDIYKIYCESFKGETHLKQIESEAQEIVNRIFTNAGV |
| 7 | pgm | A0A1X0WLC0 | MANNPRAGQPAQQSDLINVAQLTSQYYVLQPDPSNSAHAVK |
| | | | FGTSGHRGSAGRHSFNESHILAIAQAIAEVRKEQGTTGPCYVG |
| | | | KDTHALSEPAFISVLEVLTANGVDVIVQEDNGFTPTPAVSHAI |
| | | | LTHNKKGGALADGIVITPSHNPPEDGGIKYNPTNGGPADTDLT |
| | | | SVIEKRANALIAEGLKGVKRQSLDKAWQGQHLVEQDL VQPY |
| | | | VEDLVNIIDIPAIQKAGLKLGVDPLGGSGITYWKRIAEHYNLD |
| | | | LTLVNDSVDQTFRFMHLDHDGVIRMDCSSEFAMGGLLALKD |
| | | | KFDLAFANDPDYDRHGIVTPAGLMNPNHYLAVAINYLFQHRP |
| | | | QWGADVAVGKTLVSSAMIDRVVADLGRKLVEVPVGFKWFV |
| | | | DGLFDGSFGFGGEESAGASFLCFDGTPWSTDKDGIIMCLLAAE |
| | | | ITAVTGKNPQEHYNELAARFGAPSYNRIQAPATHAQKAALSK |
| | | | LSPEQVKADTLAGDPITDRLTKAPGNGASIGGLKVITKNGWF |
| | | | AARPSGTEEAYKIYCESFLGAEHREKIEKEAVDIVSKVLADAK |
| 8 | pgm | A0A4R7GJG3 | MANHPRAGQPAQQSDLINVAQLTSQYYVLQPDPANTAHAVK |
| | | | FGTSGHRGSSLRHSFNEAHILAIAQAIAEVRKEQGTTGPCYVG |
| | | | KDTHALSEPAFISVLEVLTANGVDVIVQEDNGFTPTPAVSHAI |
| | | | LTHNRNGGALADGIVITPSHNPPEDGGIKYNPTNGGPADTNLT |
| | | | SVIEKRANALLAAKLSGVKRQSLDQAWKSGHLVAKDLVQPFI |
| | | | EGLTDIVDMAAIQKAGLKLGVDPLGGSGIEYWKRIAEHYKLD |
| | | | LTLVNDEVDQTFRFMHLDHDGVIRMDCSSESAMAGLLALRD |
| | | | KFDLAFANDPDYDRHGIVTPAGLMNPNHYLAVAIQYLFQHRP |
| | | | QWGKDVAVGKTLVSSAMIDRVVEDLGRKLVEVPVGFKWFV |
| | | | DGLFDGSFGFGGEESAGASFLRFDGTPWSTDKDGIIMCLLAAE |
| | | | ITAVTGKNPQEHYNELAARFGAPSYNRIQAPATHAQKAALSK |
| | | | LSPEQVKADTLAGDPITARLTNAPGNGASIGGLKVMTKNGWF |
| | | | AARPSGTEEAYKIYCESFLGAEHREQIEKEA VEIVSK VLADAK |
| 9 | pgm | A0A5P8YI21 | MANHPRAGQPAQQSDLINVAQLTSQYYVLQPDAEDPAHAVK |
| | | | FGTSGHRGSSVRHSFNEAHILAIAQAIAEVRHQHGISGPCYVG |
| | | | KDTHALSEPAFISVLEVLTANGVDVVVQQDNGFTPTPAISHVI |
| | | | LCHNAQGKALADGIVITPSHNPPEDGGIKYNPPNGGPADTNLT |
| | | | SVIEKRANQLLSQNLKGVKRQTLDKAWQSNHLREQDLLQPY |
| | | | VEGLVDVVDIPAIQQAGLKLGVDPLGGSGIAYWQRIAEYYKL |
| | | | DLTLVNNAIDQTFRFMHLDHDGVIRMDCSSESAMAGLLALR |
| | | | DKFDLAFANDPDYDRHGIVTPAGLMNPNHYLAVSINYLFQHR |
| | | | PQWGAGVAVGKTLVSSAMIDRVVADLGRKLVEVPVGFKWF |
| | | | VDGLHDGSFGFGGEESAGASFLRFNGTPWSTDKDGIIMCLLA |
| | | | AEITAVTGKDPQRHYDDLAQRFGAPSYNRIQAPATQAQKNAL |
| | | | SRLSPEMVKANTLAGDPITARLTNAPGNGASIGGLKVMTENG |
| | | | WFAARPSGTEEAYKIYCESFLGAEHREKIEQEAVKIVSEVLAE |
| | | | AK |
| 10 | pgm | A0A6M4XLA8 | MAQHSHAGQPARLSDLTNIPRLVSAYYLNKPDMSRPEQRVAF |
| | | | GTSGHRGSALHNAFTESHILAVTQALVEYRQQAGITGPLFVG |
| | | | MDTHALSESAFASAVEVLAANGVETRIQAGLGFTPTPVISHAI |
| | | | LRHNAGKPAARADGVVITPSHNPPEDGGFKYNPPHGGPAEGE |
| | | | ITKWVEDRANAILEAGLAGVKRMPFAEALKSPFVALHDYVTP |
| | | | YVDDLKNVLDMEAIKQAGIKIGVDPLGGSGVAYWDVIAKTY |
| | | | GLNIEVVNYKVDPTFSFMTLDKDGKIRMDCSSPFAMASLIAL |
| | | | KDKFDIALGNDPDYDRHGIVTQSGLMNPNHYLAVAIQYLFTH |
| | | | RTGWSKESAVGKTLVSSSMIDRVAGEIGRTLKEVPVGFKWFV |
| | | | DGLYSGEFGFGGEESAGASFLRKDGTVWTTDKDGFILALLAA |
| | | | EILAVTGKDPQAHYDALEAKFGRSSYRRIDAPANSAQKAVLS |
| | | | KLDPALVEASTLAGEPIIAKLTKAPGNDAAIGGLKVVTENGW |
| | | | FAARPSGTESIYKIYMESFKGEAHLDLIQQEAQQIVSAALAKA |
| | | | GV |
| 11 | pgm | A0A7G9AB59 | MTDSKRAGEPAQQSDLINVAQLTAQYYVLRPEAGNAEHAVK |
| | | | FGTSGHRGSAGRHNFNEQHILAIAQAIAEDRARNGITGPCYVG |
| | | | KDTHALSEPAFISVLEVLAANGVDVIVQEHNGFTPTPAISNAIL |
| | | | VHNKKGGPLADGIVITPSHNPPEDGGIKYNPPNGGPADTNVT |
| | | | KVVENRANELLASGLQGVRRISLDAALASGHVKKQDLVQPFI |
| | | | EGLADIVDMAAIQKAGLKLGIDPLGGSGIEYWKRIAGHYKLD |
| | | | LTIVNDHVDQTFRFMHLDKDGAIRMDCSSECAMAGLLALRD |
| | | | KFDLAFANDPDYDRHGFVTPAGLMNPNHYLAVAINYLFQHR |
| | | | PQWGQDVAVGKTLVSSAMIDRVVNDLGRRLVEVPVGFKWF |
| | | | VDGLFDGSFGFGGEESAGASFLRFDGTPWSTDKDGIIMCLLA |
| | | | AEITAVTGKNPQEHYDELAARFGAPSYNRLQASATSAQKAVL |
| | | | SKLSPEMVRASQLAGDPITARLTAAPGNGASIGGLKVMTDNG |
| | | | WFAARPSGTEDAYKIYCESFLGEAHRKQIEKEAVEIVNSVLA |
| | | | AHQ |
| 12 | pgm | A0A7M2S2U2 | MAMHPRAGQKAQQEDLHNIPALVANYFLLQPDAANAEHKV |
| | | | QFGTSGHRGTADKHTFNENHILAIAQAVAEVRSEQGTTGPLF |
| | | | VGKDTHALSEPAFSSVIEVLIANGVKVIVQQDNGYTPTPGISH |
| | | | AILTYNIKHDEKADGIVITPSHNPPQDGGIKYNPTHGGPAEAE |
| | | | LTQAIEDRANELIAEGLQGVKRLPLAEAKASDLFVEMDLVKP |
| | | | YIDDLVNVIDMEAIQKANLKIGVDPLGGSGIDYWRQIGQAYN |
| | | | LDLTLVSEAIDPSFQFMSLDKDGVVRMDCSSPYAMAGLLALK |
| | | | DEYDLAFGNDPDYDRHGIVTPKGLMNPNHFLAVCIDYLYRH |
| | | | RDAWGKDVAVGKTLVSSALIDRVVADLGRALCEVPVGFKW |
| | | | FVDGLYTGKFGFGGEESAGASFLRKDGTPWSTDKDGILLCLL |
| | | | AAEITAVTGKNPQDYYEELAAKHGESKYNRIQAVANGPQKD |
| | | | VLKKLSPEMVAAETLAGDAITARLTHAPGNGAAIGGLKVTTA |
| | | | NGWFAARPSGTEDIYKIYCESFKGEEHLKQIEAEAQEIVNQVF |
| | | | AAAGL |
| 13 | pgm | A0A7W4GCM5 | MAIHQRAGQQALQTDLVNIPKLMSHYYRIIPNVEDVQQKVTF |
| | | | GTSGHRGSAFLSSFNQNHIWAITQAVVDYRHSANISGPMIVGF |
| | | | DTHALSYAAFMSVVEVLTANQVKMLIQQHDGFTPTPVVSQAI |
| | | | VRANSSAPAALIDGLIITPSHNPPQDGGIKYNPPHGGPAEGEIT |
| | | | KLIETKANEYLANQLSGVKKVNYGVAVKSGWIDEVDFIAPY |
| | | | VESLAEVIDMDAIKKANIRIGVDPLGGSGIYYWEKIAAKYGV |
| | | | DITLVNDKVDPTFGFMPLDKDGKIRMDCSSPYAMAGLLAYSE |
| | | | DYDLCVGNDPDYDRHGIVCPGFGLMNPNHYLAVAIDYLMTH |
| | | | RPQWSESLAVGKTLVSSAMIDRVCNALGRQCLEVPVGFKWF |
| | | | VAGLANSTIAFGGEESAGAAFLKRDGSTWCTDKDGFILALLA |
| | | | AEILAVTGKTPAQRYKELTDQFGESFYTRIDSPISLKKKAKFE |
| | | | QLNRDSFTDTELAGEAITAILSHAPGNNAAIGGIKVTTENGWF |
| | | | AARPSGTEALFKIYAESFISQQHLDEIIAQAQTMIDGALKG |
| 14 | pgm | A0A8A9FCS1 | MAIHSRAGQHTRQSDLINVAQLTAQYYSLKPQANQNAHRVK |
| | | | FGTSGHRGSAKRHSFNEAHILAIAQAIAEVRANNGVTGPCYV |
| | | | GKDTHALSEPAFISVLEVLAANKVNVIIQENNGYTPTPSVSFSI |
| | | | LTYNQGHQDIADGIVITPSHNPPEDGGIKYNPSNGGPADTDLT |
| | | | SVIEKRANELLENNLSGVKRLSYDDALASGYIHEQDLIMPYV |
| | | | KALGDVVDMKAIKKAGLKLGVDPLGGSGIEYWKRIGEYYEL |
| | | | DLELVNDQVDQTFRFMTLDHDGVIRMDCSSPWAMEGLLQLR |
| | | | DKFDLAFANDPDYDRHGIVTPSGLMNPNHYLAAAINYLFRHR |
| | | | PQWSNEVKVGKTLVSSAMIDRVVADLGRELVEVPVGFKWFV |
| | | | QGLFSGEFGFGGEESAGASFLRFNGKPWSTDKDGIILCLLAAE |
| | | | IKAVTGKDPQQHYNELAQRFGSPSYNRIQASATHEQKALLSR |
| | | | LSPEMVTANTLAGDPITARLTHASGNGAAIGGLKVMTDYGW |
| | | | FAARPSGTEEAYKIYCESFCGSEHRELIEKEAIEIVNNVFASKQ |
| 15 | pgm | A0A812DIK5 | MAIDKRAGQPAQQSDLINVAQLTAQYYVLKPEVGNAEHAVK |
| | | | FGTSGHRGSAARHNFNEQHILAIAQAIAEDRAKNGITGPCYVG |
| | | | KDTHALSEPAFISVLEVLAANGVDVIVQENNGFTPTPAISNAIL |
| | | | VHNKKGGPLADGIVITPSHNPPEDGGIKYNPPNGGPADTNVT |
| | | | KVVENRANELLAAGLQGVKRITLDAALASGHVKEQDLVQPF |
| | | | VEGLADIVDMAAIQKAGLTLGVDPLGGSGIEYWKRIAEHYNL |
| | | | NLTIVNDHVDQTFRFMHLDKDGAIRMDCSSECAMAGLLALR |
| | | | DKFDLAFANDPDYDRHGIVTPAGLMNPNHYLAVAINYLFQH |
| | | | RPQWGKDVAVGKTLVSSAMIDRVVNDLGRKLVEVPVGFKW |
| | | | FVDGLFDGSFGFGGEESAGASFLRFDGTPWSTDKDGIIMCLLA |
| | | | AEITAVTGKNPQQHYDELAERFGAPSYNRLQASATSAQKAAL |
| | | | SKLSPEMVSASTLAGDPITARLTAAPGNGAAIGGLKVMTDNG |
| | | | WFAARPSGTEDAYKIYCESFLGAEHRQQIEKEAVEIVSEVLKN |
| | | | A |
| 16 | pgm | A0A519EG88 | MTTRISPRAGEPAREEDLIDVSALRGAYFSLIPNSEDPQQRVRF |
| | | | GTSGHRGSSLDGSFNERHIWAITQAICDYRRSVGTTGPLFLGI |
| | | | DTHALSLPARTSALEVLAANGVDVRVAPSGQFTPTPAVSLAIL |
| | | | DFNRMHAPGPLADGIIVTPSHNPPRDGGFKYNLPHGGPADSQ |
| | | | AATAIEELANQHLVEHCKRVRRVVVARPLASGAARPYDFLQ |
| | | | RYVVALSHVIDLQAIRDSGVRIGVDPMGGAGVMYWEAIADR |
| | | | YRLPLTVVNANVDPTFSFMPLDWDGQIRMDPSSAFAMQALT |
| | | | RKCSLFDVAFACDTDHDRHGVVCPSSGLLPPNHFLSVATDYL |
| | | | LTHRPQWPAAAAVGKTVVTTSMIDRVARARDRRVLETPVGF |
| | | | KWFAQGLYDRELATACEESAGASFLRRDGSVWTTEKDGIAA |
| | | | GLLAAEITARAGADPGLYYRTLSATLGSTWSDRIDAPATREQ |
| | | | RTRLSNANAEDFQRLMLVGDEITQVQTRAQGNDQPIGGVKLT |
| | | | SRRGWVAMRPSGTEDVYKIYGESFESNEHLQKLLCDAGSIAD |
| | | | RSMADANR |
| 17 | pgm | A0A840A1Z7 | MPGQIDPRAGKPVDPAALIDVAGLLDAYFAVRPDPSDPRQRV |
| | | | AFGTSGHRGSAFTASFNEAHVQAISQAICEHRRGRGIDGPLFIG |
| | | | MDSHALSAPALRSALEVFVANEVTVMVDADDGFTPTPAVSL |
| | | | AILSYNRGRTAGLADGVVITPSHNPPQDGGFKYNPPHGGPAQ |
| | | | VEVTGPIERRANALLEGGLSSVRRRPYERARASAYVRRYDFR |
| | | | DAFVTGLAEVVDLQAIRSAGVRIGIDPLGGASASYWPLVLERF |
| | | | GLQGALVSDAVDPTFGFMTADWDGEIRMDCSSPYAMARLV |
| | | | GLRDRFDVAFGNDPDADRHGVVTPSEGLMNPNHYLVAAIAY |
| | | | LTAHRSRWSPAAGIGKTMVTTGLIDRLAASLNRPLTETPVGFR |
| | | | WFVEGLRSSRLGFAGEESAGAAFLRRDGAVWTTEKDGLSLG |
| | | | LLAAEIIARTGSDPSRLYRRVADDLGDPHYERIDAPAGPAQTA |
| | | | RLKAITAEDLELPDLAGEPVLEIETTTASGEPLGGVRVRTRSG |
| | | | WFAARPSGTEPIYKIYAESFVSGEHLRRIQQEALEAIGRVLTA |
| | | | ADQASGVDTPG |
| 18 | pgm | F2G7N9 | MALHPQAGEHAAKEQLINVAQLVSQYYSFKPDMNDPGQAVS |
| | | | FGTSGHRGTASNATFTDSHISAICQALVEYRKQEGISGPLFVG |
| | | | KDTHALSEPAMITAIEVLCANGVDVVIQRADNESMGYTPTPVI |
| | | | SRTIIRYNRAGNADKADGIVITPSHNPPSDGGFKYNPPHGGPA |
| | | | DSDVTKQIQERANALIADGNKDVQRIDIRQATARKLVREEDF |
| | | | MEPYIDDLARVIDMEAIANANLKLGTDPLGGAGVGYWSRISQ |
| | | | KYGLDITVVNDAVDPQFGFMRRDKDGKLRMDCSSAYAMAG |
| | | | LIELKDNFDLAWGNDPDFDRHGIVCKSAGLMNPNHYLAVAI |
| | | | NYLYTHRPDWPSDLNIGKTLVSSSMIDRVANSLDKPLAEMPV |
| | | | GFKWFVEGLSNSTIGFAGEESAGGIFLERNGSTWATDKDGFIL |
| | | | CLLAAEILAVTGKDPGEHYKALTEKFAAPVYSRVDVAATLEQ |
| | | | KQKLSAMDASVVTSDTLAGEPITSVQTHAPGNNAAIGGVKVS |
| | | | TENGWFAARPSGTEQIYKIYAESFNGESHLQELIAEAEALVGK |
| | | | IIK |
| 19 | pgm | P33401 | MSLLIDSVPTVAYKDQKPGTSGLRKKTKVFMDEPHYTENFIQ |
| | | | ATMQSIPNGSEGTTLVVGGDGRFYNDVIMNKIAAVGAANGV |
| | | | RKLVIGQGGLLSTPAASHIIRTYEEKCTGGGIILTASHNPGGPE |
| | | | NDLGIKYNLPNGGPAPESVTNAIWEASKKLTHYKIIKNFPKLN |
| | | | LNKLGKNQKYGPLLVDIIDPAKAYVQFLKEIFDFDLIKSFLAK |
| | | | QRKDKGWKLLFDSLNGITGPYGKAIFVDEFGLPAEEVLQNW |
| | | | HPLPDFGGLHPDPNLTYARTLVDRVDREKIAFGAASDGDGDR |
| | | | NMIYGYGPAFVSPGDSVAIIAEYAPEIPYFAKQGIYGLARSFPT |
| | | | SSAIDRVAAKKGLRCYEVPTGWKFFCALFDAKKLSICGEESF |
| | | | GTGSNHIREKDGLWAIIAWLNILAIYHRRNPEKEASIKTIQDEF |
| | | | WNEYGRTFFTRYDYEHIECEQAEKVVALLSEFVSRPNVCGSH |
| | | | FPADESLTVIDCGDFSYRDLDGSISENQGLFVKFSNGTKFVLR |
| | | | LSGTGSSGATIRLYVEKYTDKKENYGQTADVFLKPVINSIVKF |
| | | | LRFKEILGTDEPTVRT |
| 20 | pgm | P36938 | MAIHNRAGQPAQQSDLINVAQLTAQYYVLKPEAGNAEHAVK |
| | | | FGTSGHRGSAARHSFNEPHILAIAQAIAEERAKNGITGPCYVG |
| | | | KDTHALSEPAFISVLEVLAANGVDVIVQENNGFTPTPAVSNAI |
| | | | LVHNKKGGPLADGIVITPSHNPPEDGGIKYNPPNGGPADTNVT |
| | | | KVVEDRANALLADGLKGVKRISLDEAMASGHVKEQDLVQPF |
| | | | VEGLADIVDMAAIQKAGLTLGVDPLGGSGIEYWKRIGEYYNL |
| | | | NLTIVNDQVDQTFRFMHLDKDGAIRMDCSSECAMAGLLALR |
| | | | DKFDLAFANDPDYDRHGIVTPAGLMNPNHYLAVAINYLFQH |
| | | | RPQWGKDVAVGKTLVSSAMIDRVVNDLGRKLVEVPVGFKW |
| | | | FVDGLFDGSFGFGGEESAGASFLRFDGTPWSTDKDGIIMCLLA |
| | | | AEITAVTGKNPQEHYNELAKRFGAPSYNRLQAAATSAQKAA |
| | | | LSKLSPEMVSASTLAGDPITARLTAAPGNGASIGGLKVMTDN |
| | | | GWFAARPSGTEDAYKIYCESFLGEEHRKQIEKEAVEIVSEVLK |
| | | | NA |
| 21 | pgm | P37012 | MSFQIETVPTKPYEDQKPGTSGLRKKTKVFKDEPNYTENFIQS |
| | | | IMEAIPEGSKGATLVVGGDGRYYNDVILHKIAAIGAANGIKKL |
| | | | VIGQHGLLSTPAASHIMRTYEEKCTGGIILTASHNPGGPENDM |
| | | | GIKYNLSNGGPAPESVTNAIWEISKKLTSYKIIKDFPELDLGTI |
| | | | GKNKKYGPLLVDIIDITKDYVNFLKEIFDFDLIKKFIDNQRSTK |
| | | | NWKLLFDSMNGVTGPYGKAIFVDEFGLPADEVLQNWHPSPD |
| | | | FGGMHPDPNLTYASSLVKRVDREKIEFGAASDGDGDRNMIY |
| | | | GYGPSFVSPGDSVAIIAEYAAEIPYFAKQGIYGLARSFPTSGAI |
| | | | DRVAKAHGLNCYEVPTGWKFFCALFDAKKLSICGEESFGTGS |
| | | | NHVREKDGVWAIMAWLNILAIYNKHHPENEASIKTIQNEFWA |
| | | | KYGRTFFTRYDFEKVETEKANKIVDQLRAYVTKSGVVNSAFP |
| | | | ADESLKVTDCGDFSYTDLDGSVSDHQGLYVKLSNGARFVLR |
| | | | LSGTGSSGATIRLYIEKYCDDKSQYQKTAEEYLKPIINSVIKFL |
| | | | NFKQVLGTEEPTVRT |
| 22 | pgm | V5U112 | MAVNNRAGQPAQQSDLINVAQLTAQYYVLKPEVGNAEHAV |
| | | | KFGTSGHRGSAARHSFNEPHILAIAQAIAEERAKNGITGPCYV |
| | | | GKDTHALSEPAIISVLEVLAANGVDIIVQENNGYTPTPAVSNAI |
| | | | LEHNKRGGAQADGIVITPSHNPPDDGGIKYNPPNGGPADTNV |
| | | | TKVVENRANELLANGLKEVKRISLDEAWASGRIQEKDLVQPF |
| | | | IEGLAEIVDMAAIQKAGLKLGVDPLGGSGIEYWKRIAEHYKL |
| | | | NLTIVNDQVDQTFRFMHLDKDGAIRMDCSSESAMAGLLALR |
| | | | DKFDLAFANDPDYDRHGIVTPAGLMNPNHYLAVAINYLFQH |
| | | | RPQWGQDVAVGKTLVSSAMIDRVVDALGRKLVEVPVGFKW |
| | | | FVDGLYDGSFGFGGEESAGASFLRFDGTPWSTDKDGIIMCLL |
| | | | AAEITAVTGKNPQQHYDELAARFGAPSYNRLQASATSAQKA |
| | | | ALSKLSPEMVSASTLAGDPITARLTAAPGNGAAIGGLKVMTE |
| | | | NGWFAARPSGTEDAYKIYCESFLGEEHRKQIEKEAVEIVSEVL |
| | | | KNA |
| 23 | galU | EEW2752841.1 | MAAINTKVKKAVIPVAGLGTRMLPATKAIPKEMLPLVDKPLI |
| | | | QYVVNECIAAGITEIVLVTHSSKNSIENHFDTSFELEAMLEKR |
| | | | VKRQLLDEVQSICPPHVTIMQVRQGLAKGLGHAVLCAHPVV |
| | | | GDEPVAVILPDVILDEYESDLSQDNLAEMIRRFDETGHSQIMV |
| | | | EPVADVTAYGVVDCKGVELAPGESVPMVGVVEKPKADVAPS |
| | | | NLAIVGRYVLSADIWPLLAKTPPGAGDEIQLTDAIDMLIEKET |
| | | | VEAYHMKGKSHDCGNKLGYMQAFVEYGIRHNTLGTEFKAW |
| | | | LEEEMGIKK |
| 24 | galU | WP_131863470 | MSAVNTKIRKAVIPVAGLGTRMLPATKAIPKEMLPLVDKPLIQ |
| | | | YVVNECIAAGINEIILVTHSSKNSIENHFDTSFELEAMLEKRVK |
| | | | RQLLAEIQAICPPHVTIMQVRQGLAKGLGHAVMCAHPLVGDE |
| | | | PVAVILPDVIIDEFESDLKTDNLSEMIQRFSETGHSQIMVEPVE |
| | | | DVTNYGVVDCQGRELQPGDSAPMVGVVEKPEADEAPSNLAV |
| | | | VGRYVLSADIWPLLEKTPPGAGNEIQLTDAIAMLMEKETVEA |
| | | | YHLKGLSHDCGNKLGYMQAFVEYGLRHPSLGDEFTKWLKET |
| | | | IVD |
| 25 | galU | WP_046201092 | MSAKDKITKAVIPVAGLGTRMLPATKAIPKEMLPLADKPLIQ |
| | | | YVVNECITAGINEIVLVTHSSKNSIENHFDTSFELEAMLEKRV |
| | | | KRQLLGEIQSICPPHVTIMQIRQGIAKGLGHAVMCAYPLIANQ |
| | | | AVAIILPDVIIDEYEFDPTSDNLAEMLARYEESGRSQIMVKPVT |
| | | | DVTSYGIVDCQGVNMKPGDSAMIINVIEKPTISEAPSNLGIVG |
| | | | RYVLSSDIWPLLAQTPPSSSGEVQLTDSIAMLMKKETVEAYH |
| | | | LKGLSYDCGNKLGYMQAFVKYGMRHPVFGKDFTEWLEQIIL |
| | | | KKIFH |
| 26 | galU | WP_085045287 | LIQYVVNECIAAGITEIVLVTHSSKNSIENHFDTSFELEAMLEK |
| | | | RVKRQLLEEVQSICPPHVTIMQVRQGLAKGLGHAVMCAHPV |
| | | | VGNEPVAVILPDVILDEFESDLSQDNLAEMIKRFDETGRSQIM |
| | | | VEPVEDVTAYGVVDCQGAQLQPGDSVPMVGIVEKPKADVAP |
| | | | SNLAVVGRYVLGADIWPLLAKTPPGAGDEIQLTDSIAMLMDK |
| | | | ETVEAYHMKGKSHDCGNKLGYMQAFVEYGIRHNTLGEEFK |
| | | | AWLNDTVGDKK |
| 27 | galU | WP_033569991 | MSTVNKKVKKAVIPVAGLGTRMLPATKAIPKEMLPLVDKPLI |
| | | | QYVVNECIAAGINEIILVTHSSKNSIENHFDTSFELEAMLEKRV |
| | | | QRQLLKEVQAICPEHVTIMQVRQGLAKGLGHAVLCAHPLVG |
| | | | DEPVAVILPDVIIDEYESDLKKDNLSEMLQRFYSTGHSQIMVE |
| | | | PVENVSSYGVVDCKGVELKPGDSAPMVGVVEKPKASEAPSN |
| | | | LAVVGRYVLSEQIWDLLKKTEPGAGNEIQLTDAIAMLMDKET |
| | | | VEAYHLKGISHDCGNKLGYMTAFVEYGIRHDALGEDFTQWL |
| | | | KEAIEEDAN |
| 28 | galU | WP_211317651 | MQKRLKAVIPVAGLGTRMLPATKAIPKEMLPVVDKPLIQYIV |
| | | | NECVAAGVKDIVLVSHSSKNAIENHEDTSFELEAVLESRVKR |
| | | | QLLAEVQSICPSDVTVMQVRQGQAKGLGHAVLCAKPMVGN |
| | | | EPFIVMLPDVLLDDSTSDLTTENLAKMISRFEETGFSQVMVEP |
| | | | VPEQDVSKYGVADCDGVALHAGESAPMSAIVEKPKQEDAPS |
| | | | NLAVVGRYVFSADIWDLLEKTPPGAGDEIQLTDAIALLMATQ |
| | | | TVEAFHMSGKSHDCGDKLGYMKAFVQYGVRHSSEGNEFKQ |
| | | | WLKEALN |
| 29 | galU | WP_013202424 | MSAYKSKVKKAVIPVAGLGTRMLPATKAIPKEMLPLVDKPLI |
| | | | QYVVNECIAAGITEIVLVTHSSKNAIENHFDTSFELESMLEKR |
| | | | VKRQLLEEIQSICPPHVTIMQVRQGLAKGLGHAVMCAYPVVG |
| | | | DEPFAVILPDVIIDEYESNLATENLADMMRRFDETGRSQIMVE |
| | | | PVSDVTAYGVVDCQGAELSAGQSAPMVGVVEKPKADKAPS |
| | | | NLAVVGRYVLSADIWPLLAKTPPGAGDEIQLTDSIAMLMEKS |
| | | | TVEAYHLKGVSHDCGNKLGYMQAFVEYGVRHDSLGSDFKE |
| | | | WLESTVGNSK |
| 30 | galU | WP_222542068 | MSLKKVVIPVAGLGTRMLPATKAIPKEMLPVVDKPLIQYIVA |
| | | | ECVNAGFKEIILVTHASKNAIENHFDTSFELEATLEKRVKRQL |
| | | | LDEVQSICPPDVTIMHVRQGIAKGLGHAILCAQPLVGDSPFAV |
| | | | VLPDVILDEASADQRTENMAAMKRRFELSGHSQVMVEPVPE |
| | | | EEVSKYGIANCLGQELAPGEAAPMSEVVEKPARDEAPSNLAV |
| | | | VGRYLLSANIWSLLARTPAGAGDEIQLTDAIAMLMEEETVEA |
| | | | FHMTGRSHDCGDKLGYMKANVEYALRHPELGEVFKAHLAA |
| | | | LLKAQSVSE |
| 31 | galU | WP_095844465 | MSALNRKVRKAVIPVAGLGTRMLPATKAIPKEMLPLVDKPLI |
| | | | QYVVSECIAAGINEIVLVTHSSKNSIENHFDTSFELEAMLEKRV |
| | | | KRQLLDEVQSICPKGVTVMQVRQGVAKGLGHAILCAHPLVG |
| | | | DEPVAVVLPDVILDEYSANLKKDNLHEMLQRFEQTGVSQIMV |
| | | | EPVPHESVGSYGIADCKGHQLKPGESAPMVSVVEKPSPNEAP |
| | | | SNLAIVGRYVLSADIWPLLAKTPPGAGDEIQLTDAIDLLMEQE |
| | | | PVEAYHLKGVSHDCGNKLGYMKAFVEYGMRHATLGTDFSH |
| | | | WLQQVMPAGKK |
| 32 | galU | WP_166713283 | MSTSVRKIRKAVIPVAGLGTRMLPATKAIPKEMLPLVDRPLIQ |
| | | | YVVNECIAAGINEIILVTHSSKNSIENHFDTSFELEAMLEKRVK |
| | | | RQLLAEVQSICPKHVTIMQVRQGLAKGLGHAILCAYPLVGDE |
| | | | PFAVILPDVILDEYTSDLSKDNLSEMLSRYEETGHSQIMVEPVP |
| | | | MENVSNYGVADCSGVDLKAGESTPMKAVVEKPARDKAPSN |
| | | | LSIVGRYVLSPEIWPLLSKTPPGAGEEIQLTDAIAMLMKKETV |
| | | | EAYHLKGLSHDCGNKMGYMQAFVEYGMRHASLGKEFTSWL |
| | | | KSLDLNDE |
| 33 | galU | WP_218467311 | MSVIQPKVRKAIIPVAGLGTRMLPATKAIPKEMLPLVDKPLIQ |
| | | | YVVNECIAAGIHHIVLVTHSSKNSIENHEDTSFELEAILERRVK |
| | | | RQLLNEVQSICPSNVTIMHVRQGLAKGLGHAIMCAHPLVGDE |
| | | | PFAVVLPDVIIDEYESNLKKDNLSEMLNRYETTGHSQIMVEPV |
| | | | PINEVSDYGIVDCNGYGLSAGESAPIAKVVEKPSVAEAPSNLSI |
| | | | VGRYVLSADIWPLLSRIPPGAGDEIQLTDAIAMLMESEPVDAY |
| | | | HIRGRSHDCGNKMGYMQAFVEYGLRHAGLGAEFGSWLRQL |
| | | | QGTGKS |
| 34 | galU | WP_228957028 | MLPATKAIPKEMLPLVDKPLIQYVVNECIAAGINEIILVTHSSK |
| | | | NSIENHFDTSFELEAILEKRVKRQLLDEVQSICPSHVTIMQTRQ |
| | | | GIAKGLGHAVLCAKPLIGDEPFAVILPDVILDEYSTDLTRYNLS |
| | | | EMLVRYNETGASQILVEPVPHEEVSSYGIVDCQGENLQPGDS |
| | | | KLIKRVVEKPKPKEAPSNLSIVGRYVLSEKIWPLLAKTAPGAG |
| | | | DEIQLTDAIAMLIEREPVEAYHLQGVSHDCGNKLGYMQAFVR |
| | | | YGMKHHALGKEFTDWLVALQDQVKE |
| 35 | galU | WP_230432638 | MSQLQTKVHKAVIPVAGLGTRMLPATKAIPKEMLPIVDKPLI |
| | | | QYVVNECIAAGITEIVLVTHSSKNSIENHFDTSFELEATLEKRV |
| | | | KRQLLADIQSICPKHVTIMHVRQGHAKGLGHAVLCAKPLIGN |
| | | | EPFAVVLPDVIIDEYDADLHRENLAEMLQRFDDTGVSQIMVE |
| | | | PVPHEEVSSYGIADCGGAQLEPGESHAMHAVVEKPAREEAPS |
| | | | NLAIVGRYVLPARIWPLLEKTPVGAGDEIQLTDAIAALMEEQS |
| | | | VDAYHIRGRSHDCGSKLGYMQAFVEYGMRHSELGEEFTSTL |
| | | | KALLATAK |
| 36 | galU | WP_108478070 | MIKRKVRKAVIPVAGLGTRMLPATKAIPKEMLPVVDKPLIQY |
| | | | VVNECIAAGINEIVLVTHSSKNSIENHFDTSFELEAILEARVKR |
| | | | QLLDEVKSICPSHVTIMQTRQGIAKGLGHAILCAKPLVGDEPF |
| | | | AIILPDVILDRYSTDLTKFNLKEMLEHYETTGASQILVEPVPED |
| | | | DVSNYGIVDCNGVTLHPGDSKPISRMIEKPKRDEAPSNLSIVG |
| | | | RYVLSEKIWDALAKTAPGAGDEIQLTDAIALMMENKEPVEAY |
| | | | HLCGKSHDCGNKLGYMKAFVEYGMQHDTLGNDFSAWIKEL |
| | | | AANLDSSSKSN |
| 37 | galU | WP_071429960 | MSAPVSKIRKAVIPVAGLGTRMLPATKAIPKEMLPLVDKPLIQ |
| | | | YVVNECIAAGINEIVLVTHSSKNSIENHFDTSFELEAMLEKRV |
| | | | KRQLLDEIQGICPPHVTIMQVRQGEAKGLGHAVMCAHPVVG |
| | | | NEPFAVILPDVIIDEYESNPKKDNLAEMLSRYESTGRSQIMVEP |
| | | | VEDVTAYGVVDCQGATLQPGDSAPMVGVVEKPKASESPSNL |
| | | | AVVGRYVLSADIWPLLAKTPAGAGGEIQLTDSIAMMMEKET |
| | | | VEAYHLKGVSHDCGNKLGYMQAFVEYGVRHPVHGEDFKA |
| | | | WLSENVK |
| 38 | galU | WP_086938032 | MTKIRKAVIPVAGLGTRMLPATKAIPKEMLPLVDKPLIQYIVN |
| | | | ECVAAGIKEIVLVTHSSKNAIENHFDTSYELESTLEKRVKRQL |
| | | | LEEIQTICPKDVTIMHVRQGQAKGLGHAVLCAKPLVGEEPFA |
| | | | VVLPDVILDEYTADQSKENLAAMIQKFEATGESQIMVEPVAM |
| | | | EDVSKYGVADCKGATLVAGESCQMVGMVEKPKVQDAPSNL |
| | | | AVVGRYVLSKSIWEKLLVTPLGAGDEIQLTDAIAMLMEDEVV |
| | | | NAFHMTGKSHDCGDKLGYVKAFVEYAVRHDALKDDFSAYL |
| | | | KSIAAAK |
| 39 | galU | WP_049596592 | MKKNLKAVIPVAGLGTRMLPATKAIPKEMLPVVDKPLIQYIV |
| | | | NECVAAGIKDIVLVSHSSKNAIENHFDTSFELEVALESRVKRQ |
| | | | LLNEVQNICPPDVTIMQVRQGQAKGLGHAVLCAQPMVGDSP |
| | | | FIVLLPDVLLDDSTADLSRENLASMIKRFEETGHSQIMVEPVL |
| | | | QQDVSKYGIADCGNVKLQPGDSSLMTAVVEKPSVAEAPSNL |
| | | | AVVGRYVLSADIWPLLKKTPSGAGGEIQLTDAIAMLMEQQPV |
| | | | EAFHMTGKSHDCGDKLGYMKAFVTYGIRHDTEGEKFSAWL |
| | | | KQQIDA |
| 40 | ugpA | WP_021648042.1 | MFAEDLKRTEKMTVDDVFEQSAQKMREQGMSEIAISQFRHA |
| | | | YHVWASEKESAWIREDTVEPLHGVRSFHDVYKTIDHDKAVH |
| | | | AFAKTAFLKLNGGLGTSMGLQCAKSLLPVRRHKARQMRFLD |
| | | | IILGQVLTARTRLNVPLPVTFMNSFRTSDDTMKALRHQRKFK |
| | | | QTDIPLEIIQHQEPKIDAATGAPASWPANPDLEWCPPGHGDLF |
| | | | STLRESGLLDTLLEHGFEYLFISNSDNLGARPSRTLAQYFEDT |
| | | | GAPFMVEVANRTYADRKGGHIVRDTATGRLILREMSQVHPD |
| | | | DKDAAQDIAKHPYFNTNNIWVRIDVLRDMLAEHDGVLPLPVI |
| | | | INNKTVDPIDPQSPAVVQLETAMGAAIGLFEGAICVQVDRMR |
| | | | FLPVKTTNDLFIMRSDRFHLTDSYEMEDGNYIFPNVDLDPRY |
| | | | YKNIEDFNERFPYNVPSLAAANSVSIKGDWTFGRDVIMFADA |
| | | | RLEDRNEPSYVPNGEYVGPMGIEPGDWV |
| 41 | ugpA | A0A1S2W2Q8 | MDETFELSAAKMREHGMSETAINQFRHLYDVWRHEEASSWI |
| | | | REDDIEPLGHVPSFHDVYETINHDKAVDAFAKTAFLKLNGGL |
| | | | GTSMGLDKAKSLLPVRRHKAKQMRFIDIIIGQVLTARTRLDVE |
| | | | LPLTFMNSFRTSADTMKALKHHRKFSQHDVPMEIIQHQEPKL |
| | | | VAATGEPVSYPMNPELEWCPPGHGDLFSTIWESGLLDVLEER |
| | | | GFKYLFISNSDNLGARPSRTLAQHFENTGAPFMAEVAIRTKAD |
| | | | RKGGHIVRDKATGRLILREMSQVHPDDKEAAQDIAKHPYFNT |
| | | | NSIWIRIDALKDKLAECDGVLPLPVIRNKKTVNPTDPNSEQVI |
| | | | QLETAMGAAIGLFNGSICVQVDRMRFLPVKTTNDLFIMRSDR |
| | | | FHLTDTYEMEDGNYIFPNVELDPRYYKNIRDFDERFPYAVPSL |
| | | | AAANSVSIQGDWTFGRDVMMFADAKLEDKGEPSYVPNGEY |
| | | | VGPQGIEPDDWV |
| 42 | ugpA | A0A5N5RMV8 | MADVFDSCARKMREHGMSDVSIAQFRRLYDVWRNEEASSWI |
| | | | RERDVEPLIGVPSFHDVYKTIDHDKAVNAFAKTAFLKLNGGL |
| | | | GTSMGLACAKSLLPVRRHKAKQMRFLDIIVGQVLTARTRLDV |
| | | | PLPLTFMNSFRTSNDTMRVLKHNRKFRQDDIPMEIIQHSEPKI |
| | | | VAETGEPVEFAANPDLEWCPPGHGDLYSTIWESGLLDVLEEH |
| | | | GFSYLFISNSDNLGARPSRTLAQYFENTGAPFMVEVATRTEAD |
| | | | RKGGHIVRDRNTGRLLLREMSQVHPDDKADAQNIGKHPYFN |
| | | | TNSIWVRIDALKALLAAHDGVLPLPVIRNHKTVDPTDPDSTPV |
| | | | IQLETAMGAAIGLFDGAICVQVDRMRFLPVKTTNDLFIMRSD |
| | | | RFHLTDSYEMEDGNYIFPNVDLDPRYYKNIDDFNERFPYSVPS |
| | | | LAAASSVTIKGDWTFGQNVIMFSDARLEDDGEPSYVSNGKFV |
| | | | GPLGIEADDWV |
| 43 | ugpA | A0A6I1GMV9 | MNDVFEHSARKMHDKGMSDVAIAQFRHAYNVWTDAKENT |
| | | | WIREADVEPLQGVPSFHDVYETIDHDKAVQAFAKTAFLKLNG |
| | | | GLGTSMGLQCAKSLLPVRRHKAKQMRFIDIIIGQVLTARTRLD |
| | | | VPLPLTFMNSFRTSADTMKVLKHQRKFKQTEIPLEIIQHQEPKI |
| | | | DVATGEPVSWPANPGLEWCPPGHGDLFSTLWESGLLDVLHE |
| | | | HGFEYLFISNSDNLGARPSRTLAQYFENTGAPFMVEVANRTY |
| | | | ADRKGGHIVRDHATGRLILREMSQVHPDDKSCAQDIHKHPYF |
| | | | NTNNIWVRIDALRDMLAEHDGVLPLPVIINNKTVDPTDPDST |
| | | | KVVQLETAMGAAVGLFDGAICVQVDRMRFLPVKTTNDLFIM |
| | | | RSDRFHLTDSYEMEDGNYIFPNVDLDPRYYKNIEDFNEHFPY |
| | | | NVPSLAAANSVTIKGDWTFGRDVIMFADAKLDDREEPSYVPN |
| | | | GEYVGPMGIEPDDWV |
| 44 | ugpA | A0A430FTS3 | MSDVFESSAQKMRDNGMSELSISQFRHLYDVWRTEQASSWI |
| | | | REDAVEPLTGVPSFHDVYETIDHDEAVDAFAKTAFLKLNGGL |
| | | | GTSMGLECAKSLLPVRRHKAKQMRFIDIIVGQVLTARTRLGV |
| | | | PLPLTLMNSFRTSADTMRVLERNRKFHQEGIPVEIVQHQEPKI |
| | | | DAATGEPVSWPADPSLEWCPPGHGDLYSTIWESGLLDRLQER |
| | | | GFEYLFISNSDNLGARPSRTLAQHFENTGAPFMIEVATRTEAD |
| | | | RKGGHIVRDKATGRLMLREMSQVHPDDKSAAQDIAKHPYFN |
| | | | TNSIWIRIDALKEKLVEYDGVLPLPVIRNHKTVDPTDPDSTPVI |
| | | | QLETAMGAAIGLFEGAICVQVDRMRFLPVKTTDDLFIMRSDR |
| | | | FHLTDSYEMEDGNYIFPNVSLDPRYYKNIADFDERFPYNVPSL |
| | | | AAANSVTIEGDWTFGRDVIMFADAHLEDHDEPSYVPNGEYV |
| | | | GPQGIEPDDWV |
| 45 | ugpA | D6SZA7 | MDDAFELSAAKMREQGMSEIAIEQFKHLYNAWNLDKSGDKS |
| | | | AEFIRESTVEPIKSVPTFHEIYETIDHDKAVNAFAKTAFIKLNG |
| | | | GLGTSMGLSCAKSLLPVRRHKARQMRFIDIIIGQVLTARQRLG |
| | | | VNLPLIFMNSFRTSHDTLQVLKRNRKFVQDNIPVEIIQHQEPKL |
| | | | VEETGEPVSHKEDRSLEWCPPGHGDIFSTLWESDLLHTLKEN |
| | | | GIEYLFISNSDNLGARPSRTLAQHFENTGAPIMIEVAKRTQAD |
| | | | RKGGHIVRDAKTGRLMLREMTQVHPEDRRSAQSIKKHPYFN |
| | | | TNNIWVRVDALEKKLKEHKGILPLPVIFNHKTVDPTDESSTPV |
| | | | VQLETAMGAIASLFDDAICVEVDRMRFLPVKTTNDLFIMRSD |
| | | | RFHLTDSYEMEDGNYIFPDIDLDPRYYKNINDFNERFPYSVPA |
| | | | LAAAKSVTIRGDWTFGNQVSMFADAVLEDTGEPSYVPNGEF |
| | | | VGPQGIEPDSWV |
| 46 | ugpA | I4LSJ2 | MGDTFELSAAKMREQGMSEIAINQFAHLYDSWINDKSGEFIR |
| | | | ENTVEPIKSVPNFHEIYETIDHDKAVNAFAKTAFIKLNGGLGT |
| | | | SMGLSCAKSLLPVRRHKARQMRFIDIIIGQVLTARQRLGVDLP |
| | | | LIFMNSFRTSKDTLQVLKRNRKFSQKNIPVEIIQHQEPKLLEET |
| | | | CEPVSYPEDPELEWCPPGHGDIFSTIWESGLLNVLKKNGIEYL |
| | | | FISNSDNLGARPSRTLAQHFENTGASIMIEVAKRTQADRKGGH |
| | | | IVRDLETGRMMLREMTQVHPEDRRSAQSIKKHPYFNTNNIWV |
| | | | RVDALQKKLEEYNGVLPLPVICNRKTIDPTNENSEKVIQLETA |
| | | | MGAAISLFNDAICVEVDRMRFLPVKTTNDLFIMRSDRFHLTDS |
| | | | YEMEDGNYIFPDIDLDPRYYRNINDFNERFPYSVPALAAAKSV |
| | | | TIRGDWTFGNQVSMFSDAVLEDTGEPSYVPNGEFVGPQGIEP |
| | | | DEWV |
| 47 | ugpA | W5IJH5 | MTEKSLEACIEKMRSFDMGESAIGQFTRLYQSWQTDESEWIR |
| | | | ESDVEPLQGIKHIGDIHETINHRVASRALSKTAMLKLNGGLGT |
| | | | SMGLQGPKSLLPVRRHKARQMNFLDIILGQVTTVRQQQGVK |
| | | | LPLTFMNSYRTSKDSITRIRRNRNFHQDEIPLEILQNREPKIEMS |
| | | | TGLPVSYPSNPDLEWCPPGHGDVFTALWESGLLDILQAEGME |
| | | | YLFISNSDNLGARPSSTVSGAFAQSGATFMVEVARKTDADRK |
| | | | GGQIVRNKKTGCLMLREMTQVHPDDKDQATDVKVHPYFNT |
| | | | NNIWVRISALKKMLKKRKGVLPLPVIRNMKTVNPTDSSTKSV |
| | | | IQLETAMGAAISLFEDAVCVEVSRSRFLPVKTTNDLLILRSDRF |
| | | | HLTDAFEMEDGNYIFPDVNLDTRFYKNIEDFDERFPYSVPSLA |
| | | | AANSLTIEGDWTFGKDVHCFADAHLSDMGRPSYVPNGSFVG |
| | | | SQGIEPDSWQ |
| 48 | pyrE | WP_000806177.1 | MKPYQRQFIEFALSKQVLKFGEFTLKSGRKSPYFFNAGLENTG |
| | | | RDLALLGRFYAEALVDSGIEFDLLFGPAYKGIPIATTTAVALA |
| | | | EHHDLDLPYCFNRKEAKDHGEGGNLVGSALQGRVMLVDDVI |
| | | | TAGTAIRESMEIIQANGATLAGVLISLDRQERGRGEISAIQEVE |
| | | | RDYNCKVISIITLKDLIAYLEEKPEMAEHLAAVKAYREEFGV |
| 49 | pyrE | WP_066796556.1 | MSLDPQLKARLAELVKELAVVHGKVTLSSGKEADYYVDLRR |
| | | | ATLQHEASRLIGKLLRELTADWDYVAVGGLTLGADPVATSIM |
| | | | HADGRDIDAFVVRKEAKKHGMQRRIEGANIVGQKVLVVEDT |
| | | | TTTGNSPLTAVAALREAGAEVVGVATVVDRATGAADVIAAE |
| | | | GLEYRYLLDLADLGL |
| 50 | pyrF | WP_110991478.1 | MTLTASSSSRAVTNSPVVVALDYHNRDDALAFVDKIDPRDCR |
| | | | LKVGKEMFTLFGPQFVRELQQRGFDIFLDLKFHDIPNTAAHA |
| | | | VAAAADLGVWMVNVHASGGARMMTAAREALVPFGKDAPL |
| | | | LIAVTVLTSMEASDLVDLGMTLSPADYAERLAALTQKCGLD |
| | | | GVVCSAQEAVRFKQVFGQEFKLVTPGIRPQGSEAGDQRRIMT |
| | | | PEQALSAGVDYMVIGRPVTQSVDPAQTLKAINASLQRSA |
| 51 | pyrF | WP_066794162.1 | MPQSTNAQPVGFGERLVAAGQVRGRLCVGIDPHPYLLQQWG |
| | | | LEDTLDGLRRFSEACVEAFADTAALVKPQVAFYERFGSQGFA |
| | | | VLEDTLQYLREAGCLTVADAKRGDIGSTMTGYADAWLGESS |
| | | | PLRADAVTVSPYLGVAALQPVFDLAAKTGRGVFVLAATSNPE |
| | | | AVQLQNFRDDSHTVAQHVVDTCAAFNSECAASSAQDEGANQ |
| | | | HPVGDIGVVVGATLDTPPDLSQLNGPVLLPGVGAQGATAAD |
| | | | VAKITQTAPQLGFPNVSRAILNAGPQVADLQKAVQETSREYF |
| | | | DGGLV |
| 52 | pyrH | WP_000224573.1 | MATNAKPVYKRILLKLSGEALQGTEGFGIDASILDRMAQEIKE |
| | | | LVELGIQVGVVIGGGNLFRGAGLAKAGMNRVVGDHMGMLA |
| | | | TVMNGLAMRDALHRAYVNARLMSAIPLNGVCDSYSWAEAIS |
| | | | LLRNNRVVILSAGTGNPFFTTDSAACLRGIEIEADVVLKATKV |
| | | | DGVFTADPAKDPTATMYEQLTYSEVLEKELKVMDLAAFTLA |
| | | | RDHKLPIRVFNMNKPGALRRVVMGEKEGTLITE |
| 53 | pyrH | APT94782.1 | MTTESRRTAYKRVMLKLGGEMFGGGQVGIDPDVVENVARQI |
| | | | AEIARSGTEVAVVIGGGNFFRGAELSQRGMDRARSDYMGML |
| | | | GTVMNCLALQDFLGQLDVDCRVQTSINMAQVAEPYLPLRAV |
| | | | RHLEKGRVVIFGAGMGMPYFSTDTTAAQRALEIGCEVLFLAK |
| | | | GVDGVYSDDPRSNPDAELYDEITPREVIERGLKVADATAFSLC |
| | | | MDNYMPILVENLLTEGNIARAVNGERIGTLVQS |
| 54 | ndk | WP_000963837.1 | MAIERTFSIIKPNAVAKNVIGNIFARFEAAGFKIVGTKMLHLTV |
| | | | EQARGFYAEHDGKPFFDGLVEFMTSGPIVVSVLEGENAVQRH |
| | | | RDLLGATNPANALAGTLRADYADSLTENGTHGSDSVESAAR |
| | | | EIAYFFGEGEVCPRTR |
| 55 | ndk | CAF21035.1 | MTERTLILIKPDGVINGHVGEIIARIERKGLKLAALDLRVADR |
| | | | ETAEKHYEEHADKPFFGELVEFITSAPLIAGIVEGERAIDAWR |
| | | | QLAGGTDPVAKATPGTIRGDFALTVGENVVHGSDSPESAEREI |
| | | | SIWFPNL |
| 56 | adk | AAC73576.1 | MRIILLGAPGAGKGTQAQFIMEKYGIPQISTGDMLRAAVKSGS |
| | | | ELGKQAKDIMDAGKLVTDELVIALVKERIAQEDCRNGFLLDG |
| | | | FPRTIPQADAMKEAGINVDYVLEFDVPDELIVDRIVGRRVHAP |
| | | | SGRVYHVKFNPPKVEGKDDVTGEELTTRKDDQEETVRKRLV |
| | | | EYHQMTAPLIGYYSKEAEAGNTKYAKVDGTKPVAEVRADLE |
| | | | KILG |
| 57 | adk | CAF19263.1 | MRLVLLGPPGAGKGTQAAILSEKLGIPHISTGDLFRANIGEGTP |
| | | | LGIEAKQYIDAGKLVPTDVTARMVASRLAESDAAEGFLLDGF |
| | | | PRTVEQADILANLLSEAGQTLDGVVNYQVSEDVVVERMLSR |
| | | | GRADDNEETIRTRLGVYRDETAPLIDHYGDKIINIEAEGEVEEI |
| | | | NARTLKALGK |
| 58 | zwf | UGE34297.1 | MAVTQTAQACDLVIFGAKGDLARRKLLPSLYQLEKAGQLNP |
| | | | DTRIIGVGRADWDKAAYTKVVREALETFMKETIDEGLWDTLS |
| | | | ARLDFCNLDVNDTAAFSRLGAMLDQKNRITINYFAMPPSTFG |
| | | | AICKGLGEAKLNAKPARVVMEKPLGTSLATSQEINDQVGEYF |
| | | | EECQVYRIDHYLGKETVLNLLALRFANSLFVNNWDNRTIDHV |
| | | | EITVAEEVGIEGRWGYFDKAGQMRDMIQNHLLQILCMIAMSP |
| | | | PSDLSADSIRDEKVKVLKSLRRIDRSNVREKTVRGQYTAGFA |
| | | | QGKKVPGYLEEEGANKSSNTETFVAIRVDIDNWRWAGVPFY |
| | | | LRTGKRLPTKCSEVVVYFKTPELNLFKESWQDLPQNKLTIRL |
| | | | QPDEGVDIQVLNKVPGLDHKHNLQITKLDLSYSETFNQTHLA |
| | | | DAYERLLLETMRGIQALFVRRDEVEEAWKWVDSITEAWAMD |
| | | | NDAPKPYQAGTWGPVASVAMITRDGRSWNEFE |
| 59 | gnd | WP_000043484.1 | MSKQQIGVVGMAVMGRNLALNIESRGYTVSIFNRSREKTEEV |
| | | | IAENPGKKLVPYYTVKEFVESLETPRRILLMVKAGAGTDAAID |
| | | | SLKPYLDKGDIIIDGGNTFFQDTIRRNRELSAEGFNFIGTGVSG |
| | | | GEEGALKGPSIMPGGQKEAYELVAPILTKIAAVAEDGEPCVT |
| | | | YIGADGAGHYVKMVHNGIEYGDMQLIAEAYSLLKGGLNLTN |
| | | | EELAQTFTEWNNGELSSYLIDITKDIFTKKDEDGNYLVDVILD |
| | | | EAANKGTGKWTSQSALDLGEPLSLITESVFARYISSLKDQRVA |
| | | | ASKVLSGPQAQPAGDKAEFIEKVRRALYLGKIVSYAQGFSQL |
| | | | RAASEEYNWDLNYGEIAKIFRAGCIIRAQFLQKITDAYAENPQ |
| | | | IANLLLAPYFKQIADDYQQALRDVVAYAVQNGIPVPTFSAAV |
| | | | AYYDSYRAAVLPANLIQAQRDYFGAHTYKRIDKEGVFHTEW |
| | | | LD |
| 60 | prs | WP_001298109.1 | MPDMKLFAGNATPELAQRIANRLYTSLGDAAVGRFSDGEVS |
| | | | VQINENVRGGDIFIIQSTCAPTNDNLMELVVMVDALRRASAG |
| | | | RITAVIPYFGYARQDRRVRSARVPITAKVVADFLSSVGVDRVL |
| | | | TVDLHAEQIQGFFDVPVDNVFGSPILLEDMLQLNLDNPIVVSP |
| | | | DIGGVVRARAIAKLLNDTDMAIIDKRRPRANVSQVMHIIGDV |
| | | | AGRDCVLVDDMIDTGGTLCKAAEALKERGAKRVFAYATHPI |
| | | | FSGNAANNLRNSVIDEVVVCDTIPLSDEIKSLPNVRTLTLSGM |
| | | | LAEAIRRISNEESISAMFEH |
| 61 | cscB | WP_001197025.1 | MALNIPFRNAYYRFASSYSFLFFISWSLWWSLYAIWLKGHLG |
| | | | LTGTELGTLYSVNQFTSILFMMFYGIVQDKLGLKKPLIWCMSF |
| | | | ILVLTGPFMIYVYEPLLQSNFSVGLILGALFFGLGYLAGCGLL |
| | | | DSFTEKMARNFHFEYGTARAWGSFGYAIGAFFAGIFFSISPHIN |
| | | | FWLVSLFGAVFMMINMRFKDKDHQCVAADAGGVKKEDFIA |
| | | | VFKDRNFWVFVIFIVGTWSFYNIFDQQLFPVFYSGLFESHDVG |
| | | | TRLYGYLNSFQVVLEALCMAIIPFFVNRVGPKNALLIGVVIMA |
| | | | LRILSCALFVNPWIISLVKLLHAIEVPLCVISVFKYSVANFDKR |
| | | | LSSTIFLIGFQIASSLGIVLLSTPTGILFDHAGYQTVFFAISGIVC |
| | | | LMLLFGIFFLSKKREQIVMETPVPSAI |
| 62 | SuSy | WP_004872341.1 | MIEALRQQLLDDPRSWYAFLRHLVASQRDSWLYTDLQRACA |
| | | | DFREQLPEGYAEGIGPLEDFVAHTQEVIFRDPWMVFAWRPRP |
| | | | GRWIYVRIHREQLALEELSTDAYLQAKEGIVGLGAEGEAVLT |
| | | | VDFRDFRPVSRRLRDESTIGDGLTHLNRRLAGRIFSDLAAGRS |
| | | | QILEFLSLHRLDGQNLMLSNGNTDFDSLRQTVQYLGTLPRETP |
| | | | WAEIREDMRRRGFAPGWGNTAGRVRETMRLLMDLLDSPSPA |
| | | | ALESFLDRIPMISRILIVSIHGWFAQDKVLGRPDTGGQVVYILD |
| | | | QARALEREMRNRLRQQGVDVEPRILIATRLIPESDGTTCDQRL |
| | | | EPVVGAENVQILRVPFRYPDGRIHPHWISRFKIWPWLERYAQ |
| | | | DLEREVLAELGSRPDLIIGNYSDGNLVATLLSERLGVTQCNIA |
| | | | HALEKSKYLYSDLHWRDHEQDHHFACQFTADLIAMNAADII |
| | | | VTSTYQEIAGNDREIGQYEGHQDYTLPGLYRVENGIDVFDSK |
| | | | FNIVSPGADPRFYFSYARTEERPSFLEPEIESLLFGREPGADRR |
| | | | GVLEDRQKPLLLSMARMDRIKNLSGLAELYGRSSRLRGLANL |
| | | | VIIGGHVDVGNSRDAEEREEIRRMHEIMDHYQLDGQLRWVG |
| | | | ALLDKTVAGELYRVVADGRGVFVQPALFEAFGLTVIEAMSSG |
| | | | LPVFATRFGGPLEIIEDGVSGFHIDPNDHEATAERLADFLEAAR |
| | | | ERPKYWLEISDAALARVAERYTWERYAERLMTIARIFGFWRF |
| | | | VLDRESQVMERYLQMFRHLQWRPLAHAVPME |
| 63 | SPase | WP_011742626.1 | MKNKVQLITYADRLGDGTIKSMTDILRTRFDGVYDGVHILPF |
| | | | FTPFDGADAGFDPIDHTKVDERLGSWDDVAELSKTHNIMVD |
| | | | AIVNHMSWESKQFQDVLAKGEESEYYPMFLTMSSVFPNGAT |
| | | | EEDLAGIYRPRPGLPFTHYKFAGKTRLVWVSFTPQQVDIDTDS |
| | | | DKGWEYLMSIFDQMAASHVSYIRLDAVGYGAKEAGTSCFMT |
| | | | PKTFKLISRLREEGVKRGLEILIEVHSYYKKQVEIASKVDRVY |
| | | | DFALPPLLLHALSTGHVEPVAHWTDIRPNNAVTVLDTHDGIG |
| | | | VIDIGSDQLDRSLKGLVPDEDVDNLVNTIHANTHGESQAATG |
| | | | AAASNLDLYQVNSTYYSALGCNDQHYIAARAVQFFLPGVPQ |
| | | | VYYVGALAGKNDMELLRKTNNGRDINRHYYSTAEIDENLKR |
| | | | PVVKALNALAKFRNELDAFDGTFSYTTDDDTSISFTWRGETS |
| | | | QATLTFEPKRGLGVDNTTPVAMLEWEDSAGDHRSDDLIANPP |
| | | | VVA |
| 64 | galE | AAV80748.1 | MVIFVTGGAGYIGSHTILELLNNGHDVVSIDNFVNSSIESLKRV |
| | | | EQITNKKIISYQGDIRDKNLLDEIFSRHHIDAVIHFASLKSVGES |
| | | | KLKPLEYYSNNVGGTLVLLECMKRYNINKMIFSSSATVYGSN |
| | | | SIPPHTEDRRIGETTNPYGTSKFIIEIILSDYCDSDNNKSVIALRY |
| | | | FNPIGAHKSGMIGENPNGIPNNLVPYISKVAQNQLPVLNIYGN |
| | | | DYPTKDGTGVRDYIHVCDLAKGHVKALEYMFLNDVNYEAF |
| | | | NLGTGQGYSVLEIVKMFEIVTKKSIPVAICNRREGDVAESWAS |
| | | | ADLAHKKLSWKAEKNFERMIEDVWRWQINNPNGYKK |
| 65 | ugd | WP_089615770.1 | MKITISGTGYVGLSNGLLIAQNHEVVALDILPSRVAMLNDRIS |
| | | | PIVDKEIQQFLQSDKIHFNATLDKNEAYRDADYVIIATPTDYD |
| | | | PKTNYFNTSSVESVIKDVVEINPYAVMVIKSTVPVGFTAAMH |
| | | | KKYRTENIIFSPEFLREGKALYDNLHPSRIVIGERSERAERFAA |
| | | | LLQEGAIKQNIPMLFTDSTEAEAIKLFANTYLAMRVAYFNELD |
| | | | SYAESLGLNSRQIIEGVCLDPRIGNHYNNPSFGYGGYCLPKDT |
| | | | KQLLANYQSVPNNLISAIVDANRTRKDFIADAILSRKPQVVGI |
| | | | YRLIMKSGSDNFRASSIQGIMKRIKAKGVEVIIYEPVMKEDSFF |
| | | | NSRLERDLATFKQQADVIISNRMAEELKDVADKVYTRDLFGS |
| | | | D |
| 66 | glgC | WP_097472330.1 | MNAPFTYSSPTLSVEALKHSIAYKLMFTIGKDPVVANKHEWL |
| | | | NATLFAVRDRLVERWLRSNRAQLSQETRQVYYLSMEFLIGRT |
| | | | LSNAMLSLGIYEDVQGALEAMGLNLEELIDEENDPGLGNGGL |
| | | | GRLAACFLDSLATLGLPGRGYGIRYDYGMFKQNIVNGSQKES |
| | | | PDYWLEYGNPWEFKRHNTRYKVRFGGRIQQEGKKTRWIETE |
| | | | EILGVAYDQIIPGYDTDATNTLRLWSAQASSEINLGKFNQGDY |
| | | | FAAVEDKNHSENVSRVLYPDDSTYSGRELRLRQEYFLVSSTIQ |
| | | | DILSRHYQLHKTYDNLADKIAIHLNDTHPVLSIPEMMRLLIDE |
| | | | HQFSWDDAFEVCCQVFSYTNHTLMSEALETWPVDMLGKILP |
| | | | RHLQIIFEINDYFLKTLQEQYPNDTDLLGRASIIDESNGRRVRM |
| | | | AWLAVVVSHKVNGVSELHSNLMVQSLFADFAKIFPGRFTNV |
| | | | TNGVTPRRWLAVANPSLSAVLDEHLGRNWRTDLSLLNELQQ |
| | | | HCDFPMVNHAVHQAKLENKKRLAEYIAQQLNVVVNPKALF |
| | | | DVQIKRIHEYKRQLMNVLHVITRYNRIKADPDAKWVPRVNIF |
| | | | GGKAASAYYMAKHIIHLINDVAKVINNDPQIGDKLKVVFIPN |
| | | | YSVSLAQLIIPAADLSEQISLAGTEASGTSNMKFALNGALTIGT |
| | | | LDGANVEMLDHVGADNIFIFGNTAEEVEELRRQGYKPREYYE |
| | | | KDEELHQVLTQIGSGVFSPEDPGRYRDLVDSLINFGDHYQVL |
| | | | ADYRSYVDCQDKVDELYELQEEWTAKAMLNIANMGYFSSD |
| | | | RTIKEYADHIWHIDPVRL |
| 67 | opgG | WP_001343212.1 | MMKMRWLSAAVMLTLYTSSSWAFSIDDVAKQAQSLAGKGY |
| | | | ETPKSNLPSVFRDMKYADYQQIQFNHDKAYWNNLKTPFKLE |
| | | | FYHQGMYFDTPVKINEVTATAVKRIKYSPDYFTFGDVQHDK |
| | | | DTVKDLGFAGFKVLYPINSKDKNDEIVSMLGASYFRVIGAGQ |
| | | | VYGLSARGLAIDTALPSGEEFPRFKEFWIERPKPTDKRLTIYAL |
| | | | LDSPRATGAYKFVVMPGRDTVVDVQSKIYLRDKVGKLGVAP |
| | | | LTSMFLFGPNQPSPANNYRPELHDSNGLSIHAGNGEWIWRPL |
| | | | NNPKHLAVSSFSMENPQGFGLLQRGRDFSRFEDLDDRYDLRP |
| | | | SAWVTPKGEWGKGSVELVEIPTNDETNDNIVAYWTPDQLPEP |
| | | | GKEMNFKYTITFSRDEDKLHAPDNAWVQQTRRSTGDVKQSN |
| | | | LIRQPDGTIAFVVDFTGAEMKKLPEDTPVTAQTSIGDNGEIVE |
| | | | STVRYNPVTKGWRLVMRVKVKDAKKTTEMRAALVNADQTL |
| | | | SETWSYQLPANE |
| 68 | pgi | AAC76995.1 | MKNINPTQTAAWQALQKHFDEMKDVTIADLFAKDGDRFSKF |
| | | | SATFDDQMLVDYSKNRITEETLAKLQDLAKECDLAGAIKSMF |
| | | | SGEKINRTENRAVLHVALRNRSNTPILVDGKDVMPEVNAVLE |
| | | | KMKTFSEAIISGEWKGYTGKAITDVVNIGIGGSDLGPYMVTE |
| | | | ALRPYKNHLNMHFVSNVDGTHIAEVLKKVNPETTLFLVASKT |
| | | | FTTQETMTNAHSARDWFLKAAGDEKHVAKHFAALSTNAKA |
| | | | VGEFGIDTANMFEFWDWVGGRYSLWSAIGLSIVLSIGFDNFV |
| | | | ELLSGAHAMDKHFSTTPAEKNLPVLLALIGIWYNNFFGAETE |
| | | | AILPYDQYMHRFAAYFQQGNMESNGKYVDRNGNVVDYQTG |
| | | | PIIWGEPGTNGQHAFYQLIHQGTKMVPCDFIAPAITHNPLSDH |
| | | | HQKLLSNFFAQTEALAFGKSREVVEQEYRDQGKDPATLDYV |
| | | | VPFKVFEGNRPTNSILLREITPFSLGALIALYEHKIFTQGVILNIF |
| | | | TFDQWGVELGKQLANRILPELKDDKEISSHDSSTNGLINRYKA |
| | | | WRG |
| 69 | ushA | WP_000771748.1 | MKLLQRGVALALLTTFTLASETALAYEQDKTYKITVLHTNDH |
| | | | HGHFWRNEYGEYGLAAQKTLVDGIRKEVAAEGGSVLLLSGG |
| | | | DINTGVPESDLQDAEPDFRGMNLVGYDAMAIGNHEFDNPLT |
| | | | VLRQQEKWAKFPLLSANIYQKSTGERLFKPWALFKRQDLKIA |
| | | | VIGLTTDDTAKIGNPEYFTDIEFRKPADEAKLVIQELQQTEKPD |
| | | | IIIAATHMGHYDNGEHGSNAPGDVEMARALPAGSLAMIVGG |
| | | | HSQDPVCMAAENKKQVDYVPGTPCKPDQQNGIWIVQAHEW |
| | | | GKYVGRADFEFRNGEMKMVNYQLIPVNLKKKVTWEDGKSE |
| | | | RVLYTPEIAENQQMISLLSPFQNKGKAQLEVKIGETNGRLEGD |
| | | | RDKVRFVQTNMGRLILAAQMDRTGADFAVMSGGGIRDSIEA |
| | | | GDISYKNVLKVQPFGNVVVYADMTGKEVIDYLTAVAQMKP |
| | | | DSGAYPQFANVSFVAKDGKLNDLKIKGEPVDPAKTYRMATL |
| | | | NFNATGGDGYPRLDNKPGYVNTGFIDAEVLKAYIQKSSPLDV |
| | | | SVYEPKGEVSWQ |
| 70 | galK | WP_000053415.1 | MSLKEKTQSLFANAFGYPATHTIQAPGRVNLIGEHTDYNDGF |
| | | | VLPCAIDYQTVISCAPRDDRKVRVMAADYENQLDEFSLDAPI |
| | | | VAHENYQWANYVRGVVKHLQLRNNSFGGVDMVISGNVPQG |
| | | | AGLSSSASLEVAVGTVLQQLYHLPLDGAQIALNGQEAENQFV |
| | | | GCNCGIMDQLISALGKKDHALLIDCRSLGTKAVSMPKGVAV |
| | | | VIINSNFKRTLVGSEYNTRREQCETGARFFQQPALRDVTIEEFN |
| | | | AVAHELDPIVAKRVRHILTENARTVEAASALEQGDLKRMGEL |
| | | | MAESHASMRDDFEITVPQIDTLVEIVKAVIGDKGGVRMTGGG |
| | | | FGGCIVALIPEELVPAVQQAVAEQYEAKTGIKETFYVCKPSQG |
| | | | AGQC |
| 71 | galT | WP_000191497.1 | MTQFNPVDHPHRRYNPLTGQWILVSPHRAKRPWQGAQETPA |
| | | | KQVLPAHDPDCFLCAGNVRVTGDKNPDYTGTYVFINDFAAL |
| | | | MSDTPDAPESHDPLMRCQSARGTSRVICFSPDHSKTLPELSVA |
| | | | ALTEIVKTWQEQTAELGKTYPWVQVFENKGAAMGCSNPHPH |
| | | | GQIWANSFLPNEAEREDRLQKEYFAEQKSPMLVDYVQRELA |
| | | | DGSRTVVETEHWLAVVPYWAAWPFETLLLPKAHVLRITDLT |
| | | | DAQRSDLALALKKLTSRYDNLFQCSFPYSMGWHGAPFNGEE |
| | | | NQHWQLHAHFYPPLLRSATVRKFMVGYEMLAETQRDLTAE |
| | | | QAAERLRAVSDIHFRESGV |
| 72 | galU | WP_249467246.1 | MTGAFDNHSFRTAIVPAAGLGTRFLPTTKAVPKELLPVVDTP |
| | | | GIEIVASEAAQAGAKRLVIVTSPGKESVVSYFRPAPELEANLE |
| | | | RKGKTALLEKVRRGPELIEVEVAIQEEALGLGHAVAQAEPNL |
| | | | TDEDEAVAVLLPDDLVLPAGVLSKMAEVRAEHGGSVLCAFD |
| | | | IPKEQISPYGVFDVTDTGDPDVKQVHGMVEKPSPEEAPSTYA |
| | | | AAGRYLLDRAIFDALRRIEPGAGGELQLTDAVALLIEEGHPVH |
| | | | IVVHRGGRHDLGNPGGFLKAAVDFALDDPDYGPELRNWLTE |
| | | | RLGTDR |
| 73 | galU | WP_028847555.1 | MTASAEHQTFTTAIVPAAGLGTRFLPTTKSVPKELLPVVDTPA |
| | | | IELVADEARQAGAERLVIVTSPAKQSIAAYFRPAPELERSLEEK |
| | | | GKTGQLAKIRRAPELLEVEVAIQEQALGLGHAVACAEPNLGP |
| | | | EDDVVAVLLPDDLVLPHGILERMAKVRAEHGGSVLCAFDIPK |
| | | | EEISAYGVFDVSDTDDADVKRVHGMVEKPPAEQAPSTFAAA |
| | | | GRYLLDRAIFDALRRIEPGAGGELQLTDAVALLIQEGHPVHV |
| | | | VVHRGDRHDLGNPGGFLRAAVDFALQDPDYGPELRAWLTD |
| | | | RIARP |
| 74 | galU | WP_130343884.1 | MSPTSAFRTAIVPAAGLGTRFLPTTKAVPKELLPVVDTPGIEM |
| | | | VAAEAADAGASRLVIVTSPGKESVAEYFQPRPELEATLEQRG |
| | | | KTELLAKVRRATGLIEVETALQEQALGLGHAVGCAEPNLNGS |
| | | | DDAVAVLLPDDLVLPAGVLIKMSEVRAQRGGSVLCAFDIPKE |
| | | | QISAYGVFDVADTEDPDVKRVNGMVEKPPADQAPSTFAAAG |
| | | | RYLLDRAVFDALKRIEPGAGGELQLTDAIALLIAEGHPVHVV |
| | | | VHRGGRHDLGNPGGFLRAAVDFALDHPEYGPDLRSWLRNRV |
| | | | GGE |
| 75 | cscK | WP_001274885.1 | MSAKVWVLGDAVVDLLPESDGRLLPCPGGAPANVAVGIARL |
| | | | GGTSGFIGRVGDDPFGALMQRTLLTEGVDITYLKQDEWHRTS |
| | | | TVLVDLNDQGERSFTFMVRPSADLFLETTDLPCWRHGEWLH |
| | | | LCSIALSAEPSRTSAFTAMTAIRHAGGFVSFDPNIREDLWQDE |
| | | | HLLRLCLRQALQLADVVKLSEEEWRLISGKTQNDQDICALAK |
| | | | EYEIAMLLVTKGAEGVVVCYRGQVHHFAGMSVNCVDSTGA |
| | | | GDAFVAGLLTGLSSTGLSTDEREMRRIIDLAQRCGALAVTAK |
| | | | GAMTALPCRQELE |
| 76 | WaaG | AAC76655.1 | MIVAFCLYKYFPFGGLQRDFMRIASTVAARGHHVRVYTQSW |
| | | | EGDCPKAFELIQVPVKSHTNHGRNAEYYAWVQNHLKEHPAD |
| | | | RVVGFNKMPGLDVYFAADVCYAEKVAQEKGFLYRLTSRYR |
| | | | HYAAFERATFEQGKSTKLMMLTDKQIADFQKHYQTEPERFQI |
| | | | LPPGIYPDRKYSEQIPNSREIYRQKNGIKEQQNLLLQVGSDFGR |
| | | | KGVDRSIEALASLPESLRHNTLLFVVGQDKPRKFEALAEKLG |
| | | | VRSNVHFFSGRNDVSELMAAADLLLHPAYQEAAGIVLLEAIT |
| | | | AGLPVLTTAVCGYAHYIADANCGTVIAEPFSQEQLNEVLRKA |
| | | | LTQSPLRMAWAENARHYADTQDLYSLPEKAADIITGGLDG |
| 77 | otsA | AAC74966.1 | MSRLVVVSNRIAPPDEHAASAGGLAVGILGALKAAGGLWFG |
| | | | WSGETGNEDQPLKKVKKGNITWASFNLSEQDLDEYYNQFSN |
| | | | AVLWPAFHYRLDLVQFQRPAWDGYLRVNALLADKLLPLLQ |
| | | | DDDIIWIHDYHLLPFAHELRKRGVNNRIGFFLHIPFPTPEIFNAL |
| | | | PTYDTLLEQLCDYDLLGFQTENDRLAFLDCLSNLTRVTTRSA |
| | | | KSHTAWGKAFRTEVYPIGIEPKEIAKQAAGPLPPKLAQLKAEL |
| | | | KNVQNIFSVERLDYSKGLPERFLAYEALLEKYPQHHGKIRYT |
| | | | QIAPTSRGDVQAYQDIRHQLENEAGRINGKYGQLGWTPLYYL |
| | | | NQHFDRKLLMKIFRYSDVGLVTPLRDGMNLVAKEYVAAQDP |
| | | | ANPGVLVLSQFAGAANELTSALIVNPYDRDEVAAALDRALT |
| | | | MSLAERISRHAEMLDVIVKNDINHWQECFISDLKQIVPRSAES |
| | | | QQRDKVATFPKLA |
| 78 | agp | AAC74087.1 | MNKTLIAAAVAGIVLLASNAQAQTVPEGYQLQQVLMMSRHN |
| | | | LRAPLANNGSVLEQSTPNKWPEWDVPGGQLTTKGGVLEVY |
| | | | MGHYMREWLAEQGMVKSGECPPPYTVYAYANSLQRTVATA |
| | | | QFFITGAFPGCDIPVHHQEKMGTMDPTFNPVITDDSAAFSEQA |
| | | | VAAMEKELSKLQLTDSYQLLEKIVNYKDSPACKEKQQCSLV |
| | | | DGKNTFSAKYQQEPGVSGPLKVGNSLVDAFTLQYYEGFPMD |
| | | | QVAWGEIKSDQQWKVLSKLKNGYQDSLFTSPEVARNVAKPL |
| | | | VSYIDKALVTDRTSAPKITVLVGHDSNIASLLTALDFKPYQLH |
| | | | DQNERTPIGGKIVFQRWHDSKANRDLMKIEYVYQSAEQLRN |
| | | | ADALTLQAPAQRVTLELSGCPIDADGFCPMDKFDSVLNEAVK |

### INCORPORATION BY REFERENCE

References and citations to other documents, such as patents, patent applications, patent publications, journals, books, papers, web contents, publicly accessible databases, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. An engineered host cell that comprises one or more genetic modifications that result in enzymatic transformation by the engineered host cell of a carbon source through multiple chemical intermediates into a UDP-sugar, wherein the UDP-sugar is UDP-glucose or UDP-galactose.

2. (canceled)

3. (canceled)

4. The engineered host cell of claim 1, wherein the chemical intermediate is UDP-glucose and the UDP-sugar is UDP-galactose.

5. The engineered host cell of claim 1, wherein the engineered host cell is E. coli.

6. The engineered host cell of claim 5, wherein the one or more genetic modifications is overexpression of one or more genes selected from a group consisting of: (i) glucose facilitator gene (glf) or a homolog thereof, (ii) glucokinase (glk) or a homolog thereof, (iii) phosphoglucomutase (pgm) or a homolog thereof, (iv) UTP-glucose-1-phosphate-uridylyltransferase (galU) or a homolog thereof, (v) fructokinase (cscK) or a homolog thereof, and (vi) any combination thereof.

7. The engineered host cell of claim 5, wherein one or more genetic modifications is overexpression of one or more genes selected from a group consisting of: (i) sucrose transporter (cscB) or a homolog thereof, (ii) sucrose synthase (SuSy) or a homolog thereof, (iii) sucrose phosphorylase (SPase) or a homolog thereof, and (iv) any combination thereof.

8. The engineered host cell of claim 5, wherein one or more genetic modifications is downregulation or deletion of one or more genes selected from a group consisting of: (i) glucose-6-phosphate isomerase (pgi), (ii) glucose-1-phosphate adenylyltransferase (glgC), (iii) UDP-glucose 6-dehydrogenase (ugd), (iv) glucans biosynthesis glucosyltransferase (OpgG), (v) UDP-glucose 4-epimerase (galE), (vi) UDP-sugar hydrolase (ushA), (vii) UTP-glucose-1-phosphate uridylyltransferase (ugp), (viii) phosphoglucomutase (pgm), (ix) (heptosyl) lipopolysaccharide α-1,3-glucosyltransferase (WaaG), (x) trehalose-6-phosphate synthase (otsA), (xi) glucose-1-phosphatase (agp), and (xii) any combination thereof.

9. The engineered host cell of claim 6, wherein the cell is supplemented with glucose, fructose, and/or sucrose.

10. The engineered host cell of claim 1, wherein the one or more genetic modifications is overexpression of one or more genes selected from a group consisting of: (i) glucose-6-phopshate 1-dehydrogenase (zwf) or a homolog thereof; (ii) 6-phosphogluconate dehydrogenase (gnd) or a homolog thereof; (iii) phosphoribosylpyrophosphate synthetase (prs) or a homolog thereof; (iv) phosphoribosyltransferase (pyrE) or a homolog thereof; (v) orotidine 5′-phosphate decarboxylase (pyrF) or a homolog thereof, (vi) uridylate kinase (pyrH) or a homolog thereof, (vii) nucleoside diphosphate kinase (ndk) or a homolog thereof, and (viii) adenylate kinase (adk) or a homolog thereof, and (ix) any combination thereof.

11. The engineered host cell of claim 1, wherein the engineered host cell is supplemented with orotic acid.

12. The engineered host cell of claim 1, wherein the one or more genetic modifications is selected from:
overexpression of one or more genes selected from a group consisting of: glucose facilitator gene (glf) or a homolog thereof, glucokinase (glk) or a homolog thereof, phosphoglucomutase (pgm) or a homolog thereof, UTP-glucose-1-phosphate-uridylyltransferase (galU) or a homolog thereof, sucrose transporter (cscB) or a homolog thereof, sucrose synthase (SuSy) or a homolog thereof, sucrose phosphorylase (SPase) or a homolog thereof, glucose-6-phopshate 1-dehydrogenase (zwf) or a homolog thereof; 6-phosphogluconate dehydrogenase (gnd) or a homolog thereof; phosphoribosylpyrophosphate synthetase (prs) or a homolog thereof; phosphoribosyltransferase (pyrE) or a homolog thereof; orotidine 5′-phosphate decarboxylase (pyrF) or a homolog thereof, uridylate kinase (pyrH) or a homolog thereof, nucleoside diphosphate kinase (ndk) or a homolog thereof, (viii) adenylate kinase (adk) or a homolog thereof, and (ix) fructokinase (cscK) or a homolog thereof; and/or
downregulation or deletion of one or more genes selected from a group consisting of: glucose-6-phosphate isomerase (pgi), glucose-1-phosphate adenylyltransferase (glgC), UDP-glucose 6-dehydrogenase (ugd), glucans biosynthesis glucosyltransferase (OpgG), UDP-glucose 4-epimerase (galE), UDP-sugar hydrolase (ushA), UTP-glucose-1-phosphate uridylyltransferase (ugp), phosphoglucomutase (pgm), (heptosyl) lipopolysaccharide α-1,3-glucosyltransferase (WaaG), trehalose-6-phosphate synthase (otsA), glucose-1-phosphatase (agp); and/or
any combinations thereof.

13. The engineered cell of claim 1, wherein the engineered cell is the engineered host cells are supplemented with a medium comprising: orotic acid, fructose, glucose and/or sucrose.

14. The engineered host cell of claim 4, wherein the one or more genetic modification is selected from a group consisting of: (i) overexpression of UDP-glucose 4-epimerase (galE) or a homolog thereof, (ii) overexpression of galactokinase (galK) or a homolog thereof, (iii) overexpression of galactose-1-phosphate uridylyltransferase (galT) or a homolog thereof, and (iv) any combinations thereof.

15. The engineered host cell of claim 14, wherein the engineered host cells are supplemented with a medium comprising galactose.

16. The engineered host cell of claim 14, wherein the one or more genetic modifications is selected from:
overexpression of one or more genes selected from a group consisting of: glucose facilitator gene (glf) or a homolog thereof, glucokinase (glk) or a homolog thereof, phosphoglucomutase (pgm) or a homolog thereof, UTP-glucose-1-phosphate-uridylyltransferase (galU) or a homolog thereof, sucrose transporter (cscB) or a homolog thereof, sucrose synthase (SuSy) or a homolog thereof, sucrose phosphorylase (SPase) or a homolog thereof, glucose-6-phopshate 1-dehydrogenase (zwf) or a homolog thereof; 6-phosphogluconate dehydrogenase (gnd) or a homolog thereof; phosphoribosylpyrophosphate synthetase (prs) or a homolog thereof; phosphoribosyltransferase (pyrE) or a homolog thereof, orotidine 5′-phosphate decarboxylase (pyrF) or a homolog thereof, uridylate kinase (pyrH) or a homolog thereof, nucleoside diphosphate kinase (ndk) or a homolog thereof, and (viii) adenylate kinase (adk) or a homolog thereof; overexpression of UDP-glucose 4-epimerase (galE) or a homolog thereof, overexpression of galactokinase (galK) or a homolog thereof, overexpression of galactose-1-phosphate uridylyltransferase (galT) or a homolog thereof, fructokinase (cscK) or a homolog thereof; and/or
downregulation or deletion of one or more genes selected from a group consisting of: glucose-6-phosphate isomerase (pgi), glucose-1-phosphate adenylyltransferase (glgC), UDP-glucose 6-dehydrogenase (ugd), glucans biosynthesis glucosyltransferase (OpgG), UDP-sugar hydrolase (ushA), UTP-glucose-1-phosphate uridylyltransferase (ugp), phosphoglucomutase (pgm), (heptosyl) lipopolysaccharide α-1,3-glucosyltransferase (WaaG), trehalose-6-phosphate synthase (otsA), glucose-1-phosphatase (agp); and/or
any combinations thereof.

17. The engineered host cell of claim 16, wherein the engineered host cells are supplemented with a medium comprising: galactose, orotic acid, glucose, fructose, and/or sucrose.

18. The engineered host cell of claim 1, wherein the carbon source is selected from a group consisting of (i) glycerol, (ii) glucose, (iii) sucrose, (iv) galactose, (v) fructose, and (vi) any combination thereof.

19. A method of increasing the production of UDP-sugar, the method comprising: providing an engineered host cell that comprises one or more genetic modifications that result in enzymatic transformation by the engineered host cell of a carbon source through multiple chemical intermediates into a UDP-sugar, wherein the UDP-sugar is UDP-glucose or UDP-galactose.

20. (canceled)

21. (canceled)

22. The method of claim 19, wherein the chemical intermediate is UDP-glucose and the UDP-sugar is UDP-galactose.

23. The method of claim 19, wherein the engineered host cell is E. coli.

24. (canceled)

25. (canceled)

26. (canceled)

27. (canceled)

28. The method of claim 19, wherein the one or more genetic modifications is overexpression of one or more genes selected from a group consisting of: (i) glucose-6-phopshate 1-dehydrogenase (zwf) or a homolog thereof; (ii) 6-phosphogluconate dehydrogenase (gnd) or a homolog thereof; (iii) phosphoribosylpyrophosphate synthetase (prs) or a homolog thereof; (iv) phosphoribosyltransferase (pyrE) or a homolog thereof; (v) orotidine 5′-phosphate decarboxylase (pyrF) or a homolog thereof, (vi) uridylate kinase (pyrH) or a homolog thereof, (vii) nucleoside diphosphate kinase (ndk) or a homolog thereof, and (viii) adenylate kinase (adk) or a homolog thereof, and (ix) any combination thereof.

29. (canceled)

30. (canceled)

31. (canceled)

32. (canceled)

33. (canceled)

34. (canceled)

35. (canceled)

36. (canceled)


SEQUENCE LISTING (78 sequences)

SEQ ID NO: 1 - protein, 473 aa, synthetic construct
MSSESSQGLVTRLALIAAIGGLLFGYDSAVIAAIGTPVDIHFIAPRHLSATAAASLSGMVVVAVLVGCVTGSLLSGWIGIRFGRRGGLLMSSICFVAAGFGAALTEKLFGTGGSALQIFCFFRFLAGLGIGVVSTLTPTYIAEIRPPDKRGQMVSGQQMAIVTGALTGYIFTWLLAHFGSIDWVNASGWCWSPASEGLIGIAFLLLLLTAPDTPHWLVMKGRHSEASKILARLEPQADPNLTIQKIKAGFDKAMDKSSAGLFAFGITVVFAGVSVAAFQQLVGINAVLYYAPQMFQNLGFGADTALLQTISIGVVNFIFTMIASRVVDRFGRKPLLIWGALGMAAMMAVLGCCFWFKVGGVLPLASVLLYIAVFGMSWGPVCWVVLSEMFPSSIKGAAMPIAVTGQWLANILVNFLFKVADGSPALNQTFNHGFSYLVFAALSILGGLIVARFVPETKGRSLDEIEEMWRSQK

SEQ ID NO: 2 - protein, 327 aa, synthetic construct
MEIVAIDIGGTHARFSIAEVSNGRVLSLGEETTFKTAEHASLQLAWERFGEKLGRPLPRAAAIAWAGPVHGEVLKLTNNPWVLRPATLNEKLDIDTHVLINDFGAVAHAVAHMDSSYLDHICGPDEALPSDGVITILGPGTGLGVAHLLRTEGRYFVIETEGGHIDFAPLDRLEDKILARLRERFRRVSIERIISGPGLGNIYEALAAIEGVPFSLLDDIKLWQMALEGKDNLAEAALDRFCLSLGAIAGDLALAQGRTSVVIGGGVGLRIASHLPESGFRQRFVSKGRFERVMSKIPVKLITYPQPGLLGAQLPMPTNILKLNNIF

SEQ ID NO: 3 - protein, 546 aa, synthetic construct
MAIHNRAGQPAQQSDLINVAQLTAQYYVLKPEAGNAEHAVKFGTSGHRGSAARHSFNEPHILAIAQAIAEERAKNGITGPCYVGKDTHALSEPAFISVLEVLAANGVDVIVQENNGFTPTPAVSNAILVHNKKGGPLADGIVITPSHNPPEDGGIKYNPPNGGPADTNVTKVVEDRANALLADGLKGVKRISLDEAMASGHVKEQDLVQPFVEGLADIVDMAAIQKAGLTLGVDPLGGSGIEYWKRIGEYYNLNLTIVNDQVDQTFRFMHLDKDGAIRMDCSSECAMAGLLALRDKFDLAFANDPDYDRHGIVTPAGLMNPNHYLAVAINYLFQHRPQWGKDVAVGKTLVSSAMIDRVVNDLGRKLVEVPVGFKWFVDGLFDGSFGFGGEESAGASFLRFDGTPWSTDKDGIIMCLLAAEITAVTGKNPQEHYNELAKRFGAPSYNRLQAAATSAQKAALSKLSPEMVSASTLAGDPITARLTAAPGNGASIGGLKVMTDNGWFAARPSGTEDAYKIYCESFLGEEHRKQIEKEAVEIVSEVLKNA

SEQ ID NO: 4 - protein, 538 aa, synthetic construct
MAHERAGQLAQPSDLIDIAELVTAYYTRTPDADNPDQQVAFGTSGHRGSALDTAFNEAHILAITQAIVDYRAEQNTTGAIFIGRDTHALSEPAMVSALEVLLANEVEVRVDDRGRYTPTPAVSHAILTNPGTDGIVITPSHNPPRDGGFKYNPPTGGPADTDATDWIAAKANEYLRAGLEGVKRVPVSGVLDERCVKHNYLDTYVADLENVVDMKAIKEAGVRIGADPMGGASVDYWAAIAEHYGLAMTVVNPEVDGTFRFMTLDTDGKIRMDCSSPNAMASLVDNRAKYDLATGNDADADRHGIVTPDAGLMNPNHYLAVAIEYLFSHRPQWGNAGVGKTLVSSSMIDRVVANLGRELVEVPVGFKWFVPGLVDGSLGFGGEESAGASFLRFDGSVWSTDKDGIILDLLAAEILAVTGKTPSQRYAELAEEYGAPAYARTDAPANREQKAILKKLSPEQVTAMELAGEEILAKLTEAPGNGAAIGGLKVTTENAWFAARPSGTEDKYKIYAESFRGEEHLKQVQEEAQALVSQVLGS

SEQ ID NO: 5 - protein, 554 aa, synthetic construct
MAKHPRAGQQPTSCDLINVAKLTSQYYVLQPALHESAHAVKFGTSGHRGSSTKHTFNEAHILAIAQAIAEIRHAKGITGPCYVGKDTHALSEPAFISVLEVLAANQVTVIIQPENGFTPTPAISHAILTHNKSNPIHLADGIVITPSHNPPEDGGIKYNPPNGGPADTDFTGLIEKRANQLLDERSSENAVLAGVKRISLSQAISSQFIIQQDMMMPYVEDLVNVVDLSVIQQAGLKLGVDPLGGSGIAYWQQIAEYYKLNLSLVNDQVDQTFRFMPLDHDGVIRMDCSSVDAMGGLLRLKDKFDLAFANDPDYDRHGIVTPAGLMNPNHYLSVAINYLYQNRPGWPVDLGVGKTLVSSAMIDRVVSSLNRKLVEVPVGFKWFVDGLYTGKLGFGGEESAGASFLRFDAKPWSTDKDGIIMCLLSAEITARTGKNPQVHYDELSEKFGAPIYNRIQANATHEQKAILSKLSPDQVQAKTLAGDLITARITHAPSNNASIGGLKVVTDNGWFAARPSGTEEAYKIYCESFISHEHLDLISKEAVEIVDAAFASNK

SEQ ID NO: 6 - protein, 548 aa, synthetic construct
MAIHPRAGQKAQQGDLNNIPALMSDYYRLQPDPSNKNHQVEFGTSGHRGSSCKCTFNETHILAITQAIVEVRAARGIEGPLFVGKDTHALSESAFSSVVEVLIANHVGVIAQEGKSYTPTPGISHAILTFNRTYPDVLSDGIVITPSHNPPQDGGIKYNPPHGGPAEAEITQAIESRANEIIANQLKAVKRVPYESAVSSVLYVEQDLVAPYVDDLVNVIDMKAIQKANLKIGIDPLGGSGIEYWRQIAATYELNLHLVNEAVDPTFQFMTLDKDGVIRMDCSSPYAMAGLLALKDEYDLAFGNDPDYDRHGIVTPKGLMNPNHFLAVCIDYLFRHRQWDQSVAVGKTLVSSALIDRVVGDLGRELCEVPVGFKWFVEGLFHGKFGFAGEESAGASLLRFDGTPWATDKDGIILCLLAAEITAVTGKNPQQYYEALAAKHGESLYSRIQAVASASQKDVLKKMSPEMVSAETLAGDPITARLTHAPGNGAAIGGLKVTTDFGWFAARPSGTEDIYKIYCESFKGETHLKQIESEAQEIVNRIFTNAGV

SEQ ID NO: 7 - protein, 547 aa, synthetic construct
MANNPRAGQPAQQSDLINVAQLTSQYYVLQPDPSNSAHAVKFGTSGHRGSAGRHSFNESHILAIAQAIAEVRKEQGTTGPCYVGKDTHALSEPAFISVLEVLTANGVDVIVQEDNGFTPTPAVSHAILTHNKKGGALADGIVITPSHNPPEDGGIKYNPTNGGPADTDLTSVIEKRANALIAEGLKGVKRQSLDKAWQGQHLVEQDLVQPYVEDLVNIIDIPAIQKAGLKLGVDPLGGSGITYWKRIAEHYNLDLTLVNDSVDQTFRFMHLDHDGVIRMDCSSEFAMGGLLALKDKFDLAFANDPDYDRHGIVTPAGLMNPNHYLAVAINYLFQHRPQWGADVAVGKTLVSSAMIDRVVADLGRKLVEVPVGFKWFVDGLFDGSFGFGGEESAGASFLCFDGTPWSTDKDGIIMCLLAAEITAVTGKNPQEHYNELAARFGAPSYNRIQAPATHAQKAALSKLSPEQVKADTLAGDPITDRLTKAPGNGASIGGLKVITKNGWFAARPSGTEEAYKIYCESFLGAEHREKIEKEAVDIVSKVLADAK

SEQ ID NO: 8 - protein, 547 aa, synthetic construct
MANHPRAGQPAQQSDLINVAQLTSQYYVLQPDPANTAHAVKFGTSGHRGSSLRHSFNEAHILAIAQAIAEVRKEQGTTGPCYVGKDTHALSEPAFISVLEVLTANGVDVIVQEDNGFTPTPAVSHAILTHNRNGGALADGIVITPSHNPPEDGGIKYNPTNGGPADTNLTSVIEKRANALLAAKLSGVKRQSLDQAWKSGHLVAKDLVQPFIEGLTDIVDMAAIQKAGLKLGVDPLGGSGIEYWKRIAEHYKLDLTLVNDEVDQTFRFMHLDHDGVIRMDCSSESAMAGLLALRDKFDLAFANDPDYDRHGIVTPAGLMNPNHYLAVAIQYLFQHRPQWGKDVAVGKTLVSSAMIDRVVEDLGRKLVEVPVGFKWFVDGLFDGSFGFGGEESAGASFLRFDGTPWSTDKDGIIMCLLAAEITAVTGKNPQEHYNELAARFGAPSYNRIQAPATHAQKAALSKLSPEQVKADTLAGDPITARLTNAPGNGASIGGLKVMTKNGWFAARPSGTEEAYKIYCESFLGAEHREQIEKEAVEIVSKVLADAK

SEQ ID NO: 9 - protein, 547 aa, synthetic construct
MANHPRAGQPAQQSDLINVAQLTSQYYVLQPDAEDPAHAVKFGTSGHRGSSVRHSFNEAHILAIAQAIAEVRHQHGISGPCYVGKDTHALSEPAFISVLEVLTANGVDVVVQQDNGFTPTPAISHVILCHNAQGKALADGIVITPSHNPPEDGGIKYNPPNGGPADTNLTSVIEKRANQLLSQNLKGVKRQTLDKAWQSNHLREQDLLQPYVEGLVDVVDIPAIQQAGLKLGVDPLGGSGIAYWQRIAEYYKLDLTLVNNAIDQTFRFMHLDHDGVIRMDCSSESAMAGLLALRDKFDLAFANDPDYDRHGIVTPAGLMNPNHYLAVSINYLFQHRPQWGAGVAVGKTLVSSAMIDRVVADLGRKLVEVPVGFKWFVDGLHDGSFGFGGEESAGASFLRFNGTPWSTDKDGIIMCLLAAEITAVTGKDPQRHYDDLAQRFGAPSYNRIQAPATQAQKNALSRLSPEMVKANTLAGDPITARLTNAPGNGASIGGLKVMTENGWFAARPSGTEEAYKIYCESFLGAEHREKIEQEAVKIVSEVLAEAK

SEQ ID NO: 10 - protein, 549 aa, synthetic construct
MAQHSHAGQPARLSDLTNIPRLVSAYYLNKPDMSRPEQRVAFGTSGHRGSALHNAFTESHILAVTQALVEYRQQAGITGPLFVGMDTHALSESAFASAVEVLAANGVETRIQAGLGFTPTPVISHAILRHNAGKPAARADGVVITPSHNPPEDGGFKYNPPHGGPAEGEITKWVEDRANAILEAGLAGVKRMPFAEALKSPFVALHDYVTPYVDDLKNVLDMEAIKQAGIKIGVDPLGGSGVAYWDVIAKTYGLNIEVVNYKVDPTFSFMTLDKDGKIRMDCSSPFAMASLIALKDKFDIALGNDPDYDRHGIVTQSGLMNPNHYLAVAIQYLFTHRTGWSKESAVGKTLVSSSMIDRVAGEIGRTLKEVPVGFKWFVDGLYSGEFGFGGEESAGASFLRKDGTVWTTDKDGFILALLAAEILAVTGKDPQAHYDALEAKFGRSSYRRIDAPANSAQKAVLSKLDPALVEASTLAGEPIIAKLTKAPGNDAAIGGLKVVTENGWFAARPSGTESIYKIYMESFKGEAHLDLIQQEAQQIVSAALAKAGV

SEQ ID NO: 11 - protein, 547 aa, synthetic construct
MTDSKRAGEPAQQSDLINVAQLTAQYYVLRPEAGNAEHAVKFGTSGHRGSAGRHNFNEQHILAIAQAIAEDRARNGITGPCYVGKDTHALSEPAFISVLEVLAANGVDVIVQEHNGFTPTPAISNAILVHNKKGGPLADGIVITPSHNPPEDGGIKYNPPNGGPADTNVTKVVENRANELLASGLQGVRRISLDAALASGHVKKQDLVQPFIEGLADIVDMAAIQKAGLKLGIDPLGGSGIEYWKRIAGHYKLDLTIVNDHVDQTFRFMHLDKDGAIRMDCSSECAMAGLLALRDKFDLAFANDPDYDRHGFVTPAGLMNPNHYLAVAINYLFQHRPQWGQDVAVGKTLVSSAMIDRVVNDLGRRLVEVPVGFKWFVDGLFDGSFGFGGEESAGASFLRFDGTPWSTDKDGIIMCLLAAEITAVTGKNPQEHYDELAARFGAPSYNRLQASATSAQKAVLSKLSPEMVRASQLAGDPITARLTAAPGNGASIGGLKVMTDNGWFAARPSGTEDAYKIYCESFLGEAHRKQIEKEAVEIVNSVLAAHQ

SEQ ID NO: 12 - protein, 548 aa, synthetic construct
MAMHPRAGQKAQQEDLHNIPALVANYFLLQPDAANAEHKVQFGTSGHRGTADKHTFNENHILAIAQAVAEVRSEQGTTGPLFVGKDTHALSEPAFSSVIEVLIANGVKVIVQQDNGYTPTPGISHAILTYNIKHDEKADGIVITPSHNPPQDGGIKYNPTHGGPAEAELTQAIEDRANELIAEGLQGVKRLPLAEAKASDLFVEMDLVKPYIDDLVNVIDMEAIQKANLKIGVDPLGGSGIDYWRQIGQAYNLDLTLVSEAIDPSFQFMSLDKDGVVRMDCSSPYAMAGLLALKDEYDLAFGNDPDYDRHGIVTPKGLMNPNHFLAVCIDYLYRHRDAWGKDVAVGKTLVSSALIDRVVADLGRALCEVPVGFKWFVDGLYTGKFGFGGEESAGASFLRKDGTPWSTDKDGILLCLLAAEITAVTGKNPQDYYEELAAKHGESKYNRIQAVANGPQKDVLKKLSPEMVAAETLAGDAITARLTHAPGNGAAIGGLKVTTANGWFAARPSGTEDIYKIYCESFKGEEHLKQIEAEAQEIVNQVFAAAGL

SEQ ID NO: 13 - protein, 547 aa, synthetic construct
MAIHQRAGQQALQTDLVNIPKLMSHYYRIIPNVEDVQQKVTFGTSGHRGSAFLSSFNQNHIWAITQAVVDYRHSANISGPMIVGFDTHALSYAAFMSVVEVLTANQVKMLIQQHDGFTPTPVVSQAIVRANSSAPAALIDGLIITPSHNPPQDGGIKYNPPHGGPAEGEITKLIETKANEYLANQLSGVKKVNYGVAVKSGWIDEVDFIAPYVESLAEVIDMDAIKKANIRIGVDPLGGSGIYYWEKIAAKYGVDITLVNDKVDPTFGFMPLDKDGKIRMDCSSPYAMAGLLAYSEDYDLCVGNDPDYDRHGIVCPGFGLMNPNHYLAVAIDYLMTHRPQWSESLAVGKTLVSSAMIDRVCNALGRQCLEVPVGFKWFVAGLANSTIAFGGEESAGAAFLKRDGSTWCTDKDGFILALLAAEILAVTGKTPAQRYKELTDQFGESFYTRIDSPISLKKKAKFEQLNRDSFTDTELAGEAITAILSHAPGNNAAIGGIKVTTENGWFAARPSGTEALFKIYAESFISQQHLDEIIAQAQTMIDGALKG

SEQ ID NO: 14 - protein, 547 aa, synthetic construct
MAIHSRAGQHTRQSDLINVAQLTAQYYSLKPQANQNAHRVKFGTSGHRGSAKRHSFNEAHILAIAQAIAEVRANNGVTGPCYVGKDTHALSEPAFISVLEVLAANKVNVIIQENNGYTPTPSVSFSILTYNQGHQDIADGIVITPSHNPPEDGGIKYNPSNGGPADTDLTSVIEKRANELLENNLSGVKRLSYDDALASGYIHEQDLIMPYVKALGDVVDMKAIKKAGLKLGVDPLGGSGIEYWKRIGEYYELDLELVNDQVDQTFRFMTLDHDGVIRMDCSSPWAMEGLLQLRDKFDLAFANDPDYDRHGIVTPSGLMNPNHYLAAAINYLFRHRPQWSNEVKVGKTLVSSAMIDRVVADLGRELVEVPVGFKWFVQGLFSGEFGFGGEESAGASFLRFNGKPWSTDKDGIILCLLAAEIKAVTGKDPQQHYNELAQRFGSPSYNRIQASATHEQKALLSRLSPEMVTANTLAGDPITARLTHASGNGAAIGGLKVMTDYGWFAARPSGTEEAYKIYCESFCGSEHRELIEKEAIEIVNNVFASKQ

SEQ ID NO: 15 - protein, 546 aa, synthetic construct
MAIDKRAGQPAQQSDLINVAQLTAQYYVLKPEVGNAEHAVKFGTSGHRGSAARHNFNEQHILAIAQAIAEDRAKNGITGPCYVGKDTHALSEPAFISVLEVLAANGVDVIVQENNGFTPTPAISNAILVHNKKGGPLADGIVITPSHNPPEDGGIKYNPPNGGPADTNVTKVVENRANELLAAGLQGVKRITLDAALASGHVKEQDLVQPFVEGLADIVDMAAIQKAGLTLGVDPLGGSGIEYWKRIAEHYNLNLTIVNDHVDQTFRFMHLDKDGAIRMDCSSECAMAGLLALRDKFDLAFANDPDYDRHGIVTPAGLMNPNHYLAVAINYLFQHRPQWGKDVAVGKTLVSSAMIDRVVNDLGRKLVEVPVGFKWFVDGLFDGSFGFGGEESAGASFLRFDGTPWSTDKDGIIMCLLAAEITAVTGKNPQQHYDELAERFGAPSYNRLQASATSAQKAALSKLSPEMVSASTLAGDPITARLTAAPGNGAAIGGLKVMTDNGWFAARPSGTEDAYKIYCESFLGAEHRQQIEKEAVEIVSEVLKNA

SEQ ID NO: 16 - protein, 553 aa, synthetic construct
MTTRISPRAGEPAREEDLIDVSALRGAYFSLIPNSEDPQQRVRFGTSGHRGSSLDGSFNERHIWAITQAICDYRRSVGTTGPLFLGIDTHALSLPARTSALEVLAANGVDVRVAPSGQFTPTPAVSLAILDFNRMHAPGPLADGIIVTPSHNPPRDGGFKYNLPHGGPADSQAATAIEELANQHLVEHCKRVRRVVVARPLASGAARPYDFLQRYVVALSHVIDLQAIRDSGVRIGVDPMGGAGVMYWEAIADRYRLPLTVVNANVDPTFSFMPLDWDGQIRMDPSSAFAMQALTRKCSLFDVAFACDTDHDRHGVVCPSSGLLPPNHFLSVATDYLLTHRPQWPAAAAVGKTVVTTSMIDRVARARDRRVLETPVGFKWFAQGLYDRELATACEESAGASFLRRDGSVWTTEKDGIAAGLLAAEITARAGADPGLYYRTLSATLGSTWSDRIDAPATREQRTRLSNANAEDFQRLMLVGDEITQVQTRAQGNDQPIGGVKLTSRRGWVAMRPSGTEDVYKIYGESFESNEHLQKLLCDAGSIADRSMADANR

SEQ ID NO: 17 - protein, 559 aa, synthetic construct
MPGQIDPRAGKPVDPAALIDVAGLLDAYFAVRPDPSDPRQRVAFGTSGHRGSAFTASFNEAHVQAISQAICEHRRGRGIDGPLFIGMDSHALSAPALRSALEVFVANEVTVMVDADDGFTPTPAVSLAILSYNRGRTAGLADGVVITPSHNPPQDGGFKYNPPHGGPAQVEVTGPIERRANALLEGGLSSVRRRPYERARASAYVRRYDFRDAFVTGLAEVVDLQAIRSAGVRIGIDPLGGASASYWPLVLERFGLQGALVSDAVDPTFGFMTADWDGEIRMDCSSPYAMARLVGLRDRFDVAFGNDPDADRHGVVTPSEGLMNPNHYLVAAIAYLTAHRSRWSPAAGIGKTMVTTGLIDRLAASLNRPLTETPVGFRWFVEGLRSSRLGFAGEESAGAAFLRRDGAVWTTEKDGLSLGLLAAEIIARTGSDPSRLYRRVADDLGDPHYERIDAPAGPAQTARLKAITAEDLELPDLAGEPVLEIETTTASGEPLGGVRVRTRSGWFAARPSGTEPIYKIYAESFVSGEHLRRIQQEALEAIGRVLTAADQASGVDTPG

SEQ ID NO: 18 - protein, 550 aa, synthetic construct
MALHPQAGEHAAKEQLINVAQLVSQYYSFKPDMNDPGQAVSFGTSGHRGTASNATFTDSHISAICQALVEYRKQEGISGPLFVGKDTHALSEPAMITAIEVLCANGVDVVIQRADNESMGYTPTPVISRTIIRYNRAGNADKADGIVITPSHNPPSDGGFKYNPPHGGPADSDVTKQIQERANALIADGNKDVQRIDIRQATARKLVREEDFMEPYIDDLARVIDMEAIANANLKLGTDPLGGAGVGYWSRISQKYGLDITVVNDAVDPQFGFMRRDKDGKLRMDCSSAYAMAGLIELKDNFDLAWGNDPDFDRHGIVCKSAGLMNPNHYLAVAINYLYTHRPDWPSDLNIGKTLVSSSMIDRVANSLDKPLAEMPVGFKWFVEGLSNSTIGFAGEESAGGIFLERNGSTWATDKDGFILCLLAAEILAVTGKDPGEHYKALTEKFAAPVYSRVDVAATLEQKQKLSAMDASVVTSDTLAGEPITSVQTHAPGNNAAIGGVKVSTENGWFAARPSGTEQIYKIYAESFNGESHLQELIAEAEALVGKIIK

SEQ ID NO: 19 - protein, 570 aa, synthetic construct
MSLLIDSVPTVAYKDQKPGTSGLRKKTKVFMDEPHYTENFIQATMQSIPNGSEGTTLVVGGDGRFYNDVIMNKIAAVGAANGVRKLVIGQGGLLSTPAASHIIRTYEEKCTGGGIILTASHNPGGPENDLGIKYNLPNGGPAPESVTNAIWEASKKLTHYKIIKNFPKLNLNKLGKNQKYGPLLVDIIDPAKAYVQFLKEIFDFDLIKSFLAKQRKDKGWKLLFDSLNGITGPYGKAIFVDEFGLPAEEVLQNWHPLPDFGGLHPDPNLTYARTLVDRVDREKIAFGAASDGDGDRNMIYGYGPAFVSPGDSVAIIAEYAPEIPYFAKQGIYGLARSFPTSSAIDRVAAKKGLRCYEVPTGWKFFCALFDAKKLSICGEESFGTGSNHIREKDGLWAIIAWLNILAIYHRRNPEKEASIKTIQDEFWNEYGRTFFTRYDYEHIECEQAEKVVALLSEFVSRPNVCGSHFPADESLTVIDCGDFSYRDLDGSISENQGLFVKFSNGTKFVLRLSGTGSSGATIRLYVEKYTDKKENYGQTADVFLKPVINSIVKFLRFKEILGTDEPTVRT

SEQ ID NO: 20 - protein, 546 aa, synthetic construct; identical to SEQ ID NO: 3

SEQ ID NO: 21 - protein, 569 aa, synthetic construct
MSFQIETVPTKPYEDQKPGTSGLRKKTKVFKDEPNYTENFIQSIMEAIPEGSKGATLVVGGDGRYYNDVILHKIAAIGAANGIKKLVIGQHGLLSTPAASHIMRTYEEKCTGGIILTASHNPGGPENDMGIKYNLSNGGPAPESVTNAIWEISKKLTSYKIIKDFPELDLGTIGKNKKYGPLLVDIIDITKDYVNFLKEIFDFDLIKKFIDNQRSTKNWKLLFDSMNGVTGPYGKAIFVDEFGLPADEVLQNWHPSPDFGGMHPDPNLTYASSLVKRVDREKIEFGAASDGDGDRNMIYGYGPSFVSPGDSVAIIAEYAAEIPYFAKQGIYGLARSFPTSGAIDRVAKAHGLNCYEVPTGWKFFCALFDAKKLSICGEESFGTGSNHVREKDGVWAIMAWLNILAIYNKHHPENEASIKTIQNEFWAKYGRTFFTRYDFEKVETEKANKIVDQLRAYVTKSGVVNSAFPADESLKVTDCGDFSYTDLDGSVSDHQGLYVKLSNGARFVLRLSGTGSSGATIRLYIEKYCDDKSQYQKTAEEYLKPIINSVIKFLNFKQVLGTEEPTVRT

SEQ ID NO: 22 - protein, 546 aa, synthetic construct
MAVNNRAGQPAQQSDLINVAQLTAQYYVLKPEVGNAEHAVKFGTSGHRGSAARHSFNEPHILAIAQAIAEERAKNGITGPCYVGKDTHALSEPAIISVLEVLAANGVDIIVQENNGYTPTPAVSNAILEHNKRGGAQADGIVITPSHNPPDDGGIKYNPPNGGPADTNVTKVVENRANELLANGLKEVKRISLDEAWASGRIQEKDLVQPFIEGLAEIVDMAAIQKAGLKLGVDPLGGSGIEYWKRIAEHYKLNLTIVNDQVDQTFRFMHLDKDGAIRMDCSSESAMAGLLALRDKFDLAFANDPDYDRHGIVTPAGLMNPNHYLAVAINYLFQHRPQWGQDVAVGKTLVSSAMIDRVVDALGRKLVEVPVGFKWFVDGLYDGSFGFGGEESAGASFLRFDGTPWSTDKDGIIMCLLAAEITAVTGKNPQQHYDELAARFGAPSYNRLQASATSAQKAALSKLSPEMVSASTLAGDPITARLTAAPGNGAAIGGLKVMTENGWFAARPSGTEDAYKIYCESFLGEEHRKQIEKEAVEIVSEVLKNA

SEQ ID NO: 23 - protein, 302 aa, synthetic construct
MAAINTKVKKAVIPVAGLGTRMLPATKAIPKEMLPLVDKPLIQYVVNECIAAGITEIVLVTHSSKNSIENHFDTSFELEAMLEKRVKRQLLDEVQSICPPHVTIMQVRQGLAKGLGHAVLCAHPVVGDEPVAVILPDVILDEYESDLSQDNLAEMIRRFDETGHSQIMVEPVADVTAYGVVDCKGVELAPGESVPMVGVVEKPKADVAPSNLAIVGRYVLSADIWPLLAKTPPGAGDEIQLTDAIDMLIEKETVEAYHMKGKSHDCGNKLGYMQAFVEYGIRHNTLGTEFKAWLEEEMGIKK

SEQ ID NO: 24 - protein, 300 aa, synthetic construct
MSAVNTKIRKAVIPVAGLGTRMLPATKAIPKEMLPLVDKPLIQYVVNECIAAGINEIILVTHSSKNSIENHFDTSFELEAMLEKRVKRQLLAEIQAICPPHVTIMQVRQGLAKGLGHAVMCAHPLVGDEPVAVILPDVIIDEFESDLKTDNLSEMIQRFSETGHSQIMVEPVEDVTNYGVVDCQGRELQPGDSAPMVGVVEKPEADEAPSNLAVVGRYVLSADIWPLLEKTPPGAGNEIQLTDAIAMLMEKETVEAYHLKGLSHDCGNKLGYMQAFVEYGLRHPSLGDEFTKWLKETIVD

SEQ ID NO: 25 - protein, 303 aa, synthetic construct
MSAKDKITKAVIPVAGLGTRMLPATKAIPKEMLPLADKPLIQYVVNECITAGINEIVLVTHSSKNSIENHFDTSFELEAMLEKRVKRQLLGEIQSICPPHVTIMQIRQGIAKGLGHAVMCAYPLIANQAVAIILPDVIIDEYEFDPTSDNLAEMLARYEESGRSQIMVKPVTDVTSYGIVDCQGVNMKPGDSAMIINVIEKPTISEAPSNLGIVGRYVLSSDIWPLLAQTPPSSSGEVQLTDSIAMLMKKETVEAYHLKGLSYDCGNKLGYMQAFVKYGMRHPVFGKDFTEWLEQIILKKIFH

SEQ ID NO: 26 - protein, 262 aa, synthetic construct
LIQYVVNECIAAGITEIVLVTHSSKNSIENHFDTSFELEAMLEKRVKRQLLEEVQSICPPHVTIMQVRQGLAKGLGHAVMCAHPVVGNEPVAVILPDVILDEFESDLSQDNLAEMIKRFDETGRSQIMVEPVEDVTAYGVVDCQGAQLQPGDSVPMVGIVEKPKADVAPSNLAVVGRYVLGADIWPLLAKTPPGAGDEIQLTDSIAMLMDKETVEAYHMKGKSHDCGNKLGYMQAFVEYGIRHNTLGEEFKAWLNDTVGDKK

SEQ ID NO: 27 - protein, 303 aa, synthetic construct
MSTVNKKVKKAVIPVAGLGTRMLPATKAIPKEMLPLVDKPLIQYVVNECIAAGINEIILVTHSSKNSIENHFDTSFELEAMLEKRVQRQLLKEVQAICPEHVTIMQVRQGLAKGLGHAVLCAHPLVGDEPVAVILPDVIIDEYESDLKKDNLSEMLQRFYSTGHSQIMVEPVENVSSYGVVDCKGVELKPGDSAPMVGVVEKPKASEAPSNLAVVGRYVLSEQIWDLLKKTEPGAGNEIQLTDAIAMLMDKETVEAYHLKGISHDCGNKLGYMTAFVEYGIRHDALGEDFTQWLKEAIEEDAN

SEQ ID NO: 28 - protein, 297 aa, synthetic construct
MQKRLKAVIPVAGLGTRMLPATKAIPKEMLPVVDKPLIQYIVNECVAAGVKDIVLVSHSSKNAIENHFDTSFELEAVLESRVKRQLLAEVQSICPSDVTVMQVRQGQAKGLGHAVLCAKPMVGNEPFIVMLPDVLLDDSTSDLTTENLAKMISRFEETGFSQVMVEPVPEQDVSKYGVADCDGVALHAGESAPMSAIVEKPKQEDAPSNLAVVGRYVFSADIWDLLEKTPPGAGDEIQLTDAIALLMATQTVEAFHMSGKSHDCGDKLGYMKAFVQYGVRHSSEGNEFKQWLKEALN

SEQ ID NO: 29 - protein, 302 aa, synthetic construct
MSAYKSKVKKAVIPVAGLGTRMLPATKAIPKEMLPLVDKPLIQYVVNECIAAGITEIVLVTHSSKNAIENHFDTSFELESMLEKRVKRQLLEEIQSICPPHVTIMQVRQGLAKGLGHAVMCAYPVVGDEPFAVILPDVIIDEYESNLATENLADMMRRFDETGRSQIMVEPVSDVTAYGVVDCQGAELSAGQSAPMVGVVEKPKADKAPSNLAVVGRYVLSADIWPLLAKTPPGAGDEIQLTDSIAMLMEKSTVEAYHLKGVSHDCGNKLGYMQAFVEYGVRHDSLGSDFKEWLESTVGNSK

SEQ ID NO: 30 - protein, 302 aa, synthetic construct
MSLKKVVIPVAGLGTRMLPATKAIPKEMLPVVDKPLIQYIVAECVNAGFKEIILVTHASKNAIENHFDTSFELEATLEKRVKRQLLDEVQSICPPDVTIMHVRQGIAKGLGHAILCAQPLVGDSPFAVVLPDVILDEASADQRTENMAAMKRRFELSGHSQVMVEPVPEEEVSKYGIANCLGQELAPGEAAPMSEVVEKPARDEAPSNLAVVGRYLLSANIWSLLARTPAGAGDEIQLTDAIAMLMEEETVEAFHMTGRSHDCGDKLGYMKANVEYALRHPELGEVFKAHLAALLKAQSVSE

SEQ ID NO: 31 - protein, 305 aa, synthetic construct
MSALNRKVRKAVIPVAGLGTRMLPATKAIPKEMLPLVDKPLIQYVVSECIAAGINEIVLVTHSSKNSIENHFDTSFELEAMLEKRVKRQLLDEVQSICPKGVTVMQVRQGVAKGLGHAILCAHPLVGDEPVAVVLPDVILDEYSANLKKDNLHEMLQRFEQTGVSQIMVEPVPHESVGSYGIADCKGHQLKPGESAPMVSVVEKPSPNEAPSNLAIVGRYVLSADIWPLLAKTPPGAGDEIQLTDAIDLLMEQEPVEAYHLKGVSHDCGNKLGYMKAFVEYGMRHATLGTDFSHWLQQVMPAGKK

SEQ ID NO: 32 - protein, 304 aa, synthetic construct
MSTSVRKIRKAVIPVAGLGTRMLPATKAIPKEMLPLVDRPLIQYVVNECIAAGINEIILVTHSSKNSIENHFDTSFELEAMLEKRVKRQLLAEVQSICPKHVTIMQVRQGLAKGLGHAILCAYPLVGDEPFAVILPDVILDEYTSDLSKDNLSEMLSRYEETGHSQIMVEPVPMENVSNYGVADCSGVDLKAGESTPMKAVVEKPARDKAPSNLSIVGRYVLSPEIWPLLSKTPPGAGEEIQLTDAIAMLMKKETVEAYHLKGLSHDCGNKMGYMQAFVEYGMRHASLGKEFTSWLKSLDLNDE

SEQ ID NO: 33 - protein, 305 aa, synthetic construct
MSVIQPKVRKAIIPVAGLGTRMLPATKAIPKEMLPLVDKPLIQYVVNECIAAGIHHIVLVTHSSKNSIENHFDTSFELEAILERRVKRQLLNEVQSICPSNVTIMHVRQGLAKGLGHAIMCAHPLVGDEPFAVVLPDVIIDEYESNLKKDNLSEMLNRYETTGHSQIMVEPVPINEVSDYGIVDCNGYGLSAGESAPIAKVVEKPSVAEAPSNLSIVGRYVLSADIWPLLSRIPPGAGDEIQLTDAIAMLMESEPVDAYHIRGRSHDCGNKMGYMQAFVEYGLRHAGLGAEFGSWLRQLQGTGKS

SEQ ID NO: 34 - protein, 284 aa, synthetic construct
MLPATKAIPKEMLPLVDKPLIQYVVNECIAAGINEIILVTHSSKNSIENHFDTSFELEAILEKRVKRQLLDEVQSICPSHVTIMQTRQGIAKGLGHAVLCAKPLIGDEPFAVILPDVILDEYSTDLTRYNLSEMLVRYNETGASQILVEPVPHEEVSSYGIVDCQGENLQPGDSKLIKRVVEKPKPKEAPSNLSIVGRYVLSEKIWPLLAKTAPGAGDEIQLTDAIAMLIEREPVEAYHLQGVSHDCGNKLGYMQAFVRYGMKHHALGKEFTDWLVALQDQVKE

SEQ ID NO: 35 - protein, 304 aa, synthetic construct
MSQLQTKVHKAVIPVAGLGTRMLPATKAIPKEMLPIVDKPLIQYVVNECIAAGITEIVLVTHSSKNSIENHFDTSFELEATLEKRVKRQLLADIQSICPKHVTIMHVRQGHAKGLGHAVLCAKPLIGNEPFAVVLPDVIIDEYDADLHRENLAEMLQRFDDTGVSQIMVEPVPHEEVSSYGIADCGGAQLEPGESHAMHAVVEKPAREEAPSNLAIVGRYVLPARIWPLLEKTPVGAGDEIQLTDAIAALMEEQSVDAYHIRGRSHDCGSKLGYMQAFVEYGMRHSELGEEFTSTLKALLATAK

SEQ ID NO: 36 - protein, 309 aa, synthetic construct
MIKRKVRKAVIPVAGLGTRMLPATKAIPKEMLPVVDKPLIQYVVNECIAAGINEIVLVTHSSKNSIENHFDTSFELEAILEARVKRQLLDEVKSICPSHVTIMQTRQGIAKGLGHAILCAKPLVGDEPFAIILPDVILDRYSTDLTKFNLKEMLEHYETTGASQILVEPVPEDDVSNYGIVDCNGVTLHPGDSKPISRMIEKPKRDEAPSNLSIVGRYVLSEKIWDALAKTAPGAGDEIQLTDAIALMMENKEPVEAYHLCGKSHDCGNKLGYMKAFVEYGMQHDTLGNDFSAWIKELAANLDSSSKSN

SEQ ID NO: 37 - protein, 299 aa, synthetic construct
MSAPVSKIRKAVIPVAGLGTRMLPATKAIPKEMLPLVDKPLIQYVVNECIAAGINEIVLVTHSSKNSIENHFDTSFELEAMLEKRVKRQLLDEIQGICPPHVTIMQVRQGEAKGLGHAVMCAHPVVGNEPFAVILPDVIIDEYESNPKKDNLAEMLSRYESTGRSQIMVEPVEDVTAYGVVDCQGATLQPGDSAPMVGVVEKPKASESPSNLAVVGRYVLSADIWPLLAKTPAGAGGEIQLTDSIAMMMEKETVEAYHLKGVSHDCGNKLGYMQAFVEYGVRHPVHGEDFKAWLSENVK

SEQ ID NO: 38 - protein, 299 aa, synthetic construct
MTKIRKAVIPVAGLGTRMLPATKAIPKEMLPLVDKPLIQYIVNECVAAGIKEIVLVTHSSKNAIENHFDTSYELESTLEKRVKRQLLEEIQTICPKDVTIMHVRQGQAKGLGHAVLCAKPLVGEEPFAVVLPDVILDEYTADQSKENLAAMIQKFEATGESQIMVEPVAMEDVSKYGVADCKGATLVAGESCQMVGMVEKPKVQDAPSNLAVVGRYVLSKSIWEKLLVTPLGAGDEIQLTDAIAMLMEDEVVNAFHMTGKSHDCGDKLGYVKAFVEYAVRHDALKDDFSAYLKSIAAAK

SEQ ID NO: 39 - protein, 298 aa, synthetic construct
MKKNLKAVIPVAGLGTRMLPATKAIPKEMLPVVDKPLIQYIVNECVAAGIKDIVLVSHSSKNAIENHFDTSFELEVALESRVKRQLLNEVQNICPPDVTIMQVRQGQAKGLGHAVLCAQPMVGDSPFIVLLPDVLLDDSTADLSRENLASMIKRFEETGHSQIMVEPVLQQDVSKYGIADCGNVKLQPGDSSLMTAVVEKPSVAEAPSNLAVVGRYVLSADIWPLLKKTPSGAGGEIQLTDAIAMLMEQQPVEAFHMTGKSHDCGDKLGYMKAFVTYGIRHDTEGEKFSAWLKQQIDA

SEQ ID NO: 40 - protein, 488 aa, synthetic construct
MFAEDLKRTEKMTVDDVFEQSAQKMREQGMSEIAISQFRHAYHVWASEKESAWIREDTVEPLHGVRSFHDVYKTIDHDKAVHAFAKTAFLKLNGGLGTSMGLQCAKSLLPVRRHKARQMRFLDIILGQVLTARTRLNVPLPVTFMNSFRTSDDTMKALRHQRKFKQTDIPLEIIQHQEPKIDAATGAPASWPANPDLEWCPPGHGDLFSTLRESGLLDTLLEHGFEYLFISNSDNLGARPSRTLAQYFEDTGAPFMVEVANRTYADRKGGHIVRDTATGRLILREMSQVHPDDKDAAQDIAKHPYFNTNNIWVRIDVLRDMLAEHDGVLPLPVIINNKTVDPIDPQSPAVVQLETAMGAAIGLFEGAICVQVDRMRFLPVKTTNDLFIMRSDRFHLTDSYEMEDGNYIFPNVDLDPRYYKNIEDFNERFPYNVPSLAAANSVSIKGDWTFGRDVIMFADARLEDRNEPSYVPNGEYVGPMGIEPGDWV

SEQ ID NO: 41 - protein, 475 aa, synthetic construct
MDETFELSAAKMREHGMSETAINQFRHLYDVWRHEEASSWIREDDIEPLGHVPSFHDVYETINHDKAVDAFAKTAFLKLNGGLGTSMGLDKAKSLLPVRRHKAKQMRFIDIIIGQVLTARTRLDVELPLTFMNSFRTSADTMKALKHHRKFSQHDVPMEIIQHQEPKLVAATGEPVSYPMNPELEWCPPGHGDLFSTIWESGLLDVLEERGFKYLFISNSDNLGARPSRTLAQHFENTGAPFMAEVAIRTKADRKGGHIVRDKATGRLILREMSQVHPDDKEAAQDIAKHPYFNTNSIWIRIDALKDKLAECDGVLPLPVIRNKKTVNPTDPNSEQVIQLETAMGAAIGLFNGSICVQVDRMRFLPVKTTNDLFIMRSDRFHLTDTYEMEDGNYIFPNVELDPRYYKNIRDFDERFPYAVPSLAAANSVSIQGDWTFGRDVMMFADAKLEDKGEPSYVPNGEYVGPQGIEPDDWV

SEQ ID NO: 42 - protein, 475 aa, synthetic construct
MADVFDSCARKMREHGMSDVSIAQFRRLYDVWRNEEASSWIRERDVEPLIGVPSFHDVYKTIDHDKAVNAFAKTAFLKLNGGLGTSMGLACAKSLLPVRRHKAKQMRFLDIIVGQVLTARTRLDVPLPLTFMNSFRTSNDTMRVLKHNRKFRQDDIPMEIIQHSEPKIVAETGEPVEFAANPDLEWCPPGHGDLYSTIWESGLLDVLEEHGFSYLFISNSDNLGARPSRTLAQYFENTGAPFMVEVATRTEADRKGGHIVRDRNTGRLLLREMSQVHPDDKADAQNIGKHPYFNTNSIWVRIDALKALLAAHDGVLPLPVIRNHKTVDPTDPDSTPVIQLETAMGAAIGLFDGAICVQVDRMRFLPVKTTNDLFIMRSDRFHLTDSYEMEDGNYIFPNVDLDPRYYKNIDDFNERFPYSVPSLAAASSVTIKGDWTFGQNVIMFSDARLEDDGEPSYVSNGKFVGPLGIEADDWV

SEQ ID NO: 43 - protein, 475 aa, synthetic construct
MNDVFEHSARKMHDKGMSDVAIAQFRHAYNVWTDAKENTWIREADVEPLQGVPSFHDVYETIDHDKAVQAFAKTAFLKLNGGLGTSMGLQCAKSLLPVRRHKAKQMRFIDIIIGQVLTARTRLDVPLPLTFMNSFRTSADTMKVLKHQRKFKQTEIPLEIIQHQEPKIDVATGEPVSWPANPGLEWCPPGHGDLFSTLWESGLLDVLHEHGFEYLFISNSDNLGARPSRTLAQYFENTGAPFMVEVANRTYADRKGGHIVRDHATGRLILREMSQVHPDDKSCAQDIHKHPYFNTNNIWVRIDALRDMLAEHDGVLPLPVIINNKTVDPTDPDSTKVVQLETAMGAAVGLFDGAICVQVDRMRFLPVKTTNDLFIMRSDRFHLTDSYEMEDGNYIFPNVDLDPRYYKNIEDFNEHFPYNVPSLAAANSVTIKGDWTFGRDVIMFADAKLDDREEPSYVPNGEYVGPMGIEPDDWV

SEQ ID NO: 44 - protein, 475 aa, synthetic construct
MSDVFESSAQKMRDNGMSELSISQFRHLYDVWRTEQASSWIREDAVEPLTGVPSFHDVYETIDHDEAVDAFAKTAFLKLNGGLGTSMGLECAKSLLPVRRHKAKQMRFIDIIVGQVLTARTRLGVPLPLTLMNSFRTSADTMRVLERNRKFHQEGIPVEIVQHQEPKIDAATGEPVSWPADPSLEWCPPGHGDLYSTIWESGLLDRLQERGFEYLFISNSDNLGARPSRTLAQHFENTGAPFMIEVATRTEADRKGGHIVRDKATGRLMLREMSQVHPDDKSAAQDIAKHPYFNTNSIWIRIDALKEKLVEYDGVLPLPVIRNHKTVDPTDPDSTPVIQLETAMGAAIGLFEGAICVQVDRMRFLPVKTTDDLFIMRSDRFHLTDSYEMEDGNYIFPNVSLDPRYYKNIADFDERFPYNVPSLAAANSVTIEGDWTFGRDVIMFADAHLEDHDEPSYVPNGEYVGPQGIEPDDWV

SEQ ID NO: 45 - protein, 479 aa, synthetic construct
MDDAFELSAAKMREQGMSEIAIEQFKHLYNAWNLDKSGDKSAEFIRESTVEPIKSVPTFHEIYETIDHDKAVNAFAKTAFIKLNGGLGTSMGLSCAKSLLPVRRHKARQMRFIDIIIGQVLTARQRLGVNLPLIFMNSFRTSHDTLQVLKRNRKFVQDNIPVEIIQHQEPKLVEETGEPVSHKEDRSLEWCPPGHGDIFSTLWESDLLHTLKENGIEYLFISNSDNLGARPSRTLAQHFENTGAPIMIEVAKRTQADRKGGHIVRDAKTGRLMLREMTQVHPEDRRSAQSIKKHPYFNTNNIWVRVDALEKKLKEHKGILPLPVIFNHKTVDPTDESSTPVVQLETAMGAIASLFDDAICVEVDRMRFLPVKTTNDLFIMRSDRFHLTDSYEMEDGNYIFPDIDLDPRYYKNINDFNERFPYSVPALAAAKSVTIRGDWTFGNQVSMFADAVLEDTGEPSYVPNGEFVGPQGIEPDSWV

SEQ ID NO: 46 - protein, 475 aa, synthetic construct
MGDTFELSAAKMREQGMSEIAINQFAHLYDSWINDKSGEFIRENTVEPIKSVPNFHEIYETIDHDKAVNAFAKTAFIKLNGGLGTSMGLSCAKSLLPVRRHKARQMRFIDIIIGQVLTARQRLGVDLPLIFMNSFRTSKDTLQVLKRNRKFSQKNIPVEIIQHQEPKLLEETCEPVSYPEDPELEWCPPGHGDIFSTIWESGLLNVLKKNGIEYLFISNSDNLGARPSRTLAQHFENTGASIMIEVAKRTQADRKGGHIVRDLETGRMMLREMTQVHPEDRRSAQSIKKHPYFNTNNIWVRVDALQKKLEEYNGVLPLPVICNRKTIDPTNENSEKVIQLETAMGAAISLFNDAICVEVDRMRFLPVKTTNDLFIMRSDRFHLTDSYEMEDGNYIFPDIDLDPRYYRNINDFNERFPYSVPALAAAKSVTIRGDWTFGNQVSMFSDAVLEDTGEPSYVPNGEFVGPQGIEPDEWV

SEQ ID NO: 47 - protein, 475 aa, synthetic construct
MTEKSLEACIEKMRSFDMGESAIGQFTRLYQSWQTDESEWIRESDVEPLQGIKHIGDIHETINHRVASRALSKTAMLKLNGGLGTSMGLQGPKSLLPVRRHKARQMNFLDIILGQVTTVRQQQGVKLPLTFMNSYRTSKDSITRIRRNRNFHQDEIPLEILQNREPKIEMSTGLPVSYPSNPDLEWCPPGHGDVFTALWESGLLDILQAEGMEYLFISNSDNLGARPSSTVSGAFAQSGATFMVEVARKTDADRKGGQIVRNKKTGCLMLREMTQVHPDDKDQATDVKVHPYFNTNNIWVRISALKKMLKKRKGVLPLPVIRNMKTVNPTDSSTKSVIQLETAMGAAISLFEDAVCVEVSRSRFLPVKTTNDLLILRSDRFHLTDAFEMEDGNYIFPDVNLDTRFYKNIEDFDERFPYSVPSLAAANSLTIEGDWTFGKDVHCFADAHLSDMGRPSYVPNGSFVGSQGIEPDSWQ

SEQ ID NO: 48 - protein, 213 aa, synthetic construct
MKPYQRQFIEFALSKQVLKFGEFTLKSGRKSPYFFNAGLFNTGRDLALLGRFYAEALVDSGIEFDLLFGPAYKGIPIATTTAVALAEHHDLDLPYCFNRKEAKDHGEGGNLVGSALQGRVMLVDDVITAGTAIRESMEIIQANGATLAGVLISLDRQERGRGEISAIQEVERDYNCKVISIITLKDLIAYLEEKPEMAEHLAAVKAYREEFGV

SEQ ID NO: 49 - protein, 180 aa, synthetic construct
MSLDPQLKARLAELVKELAVVHGKVTLSSGKEADYYVDLRRATLQHEASRLIGKLLRELTADWDYVAVGGLTLGADPVATSIMHADGRDIDAFVVRKEAKKHGMQRRIEGANIVGQKVLVVEDTTTTGNSPLTAVAALREAGAEVVGVATVVDRATGAADVIAAEGLEYRYLLDLADLGL

SEQ ID NO: 50 - protein, 245 aa, synthetic construct
MTLTASSSSRAVTNSPVVVALDYHNRDDALAFVDKIDPRDCRLKVGKEMFTLFGPQFVRELQQRGFDIFLDLKFHDIPNTAAHAVAAAADLGVWMVNVHASGGARMMTAAREALVPFGKDAPLLIAVTVLTSMEASDLVDLGMTLSPADYAERLAALTQKCGLDGVVCSAQEAVRFKQVFGQEFKLVTPGIRPQGSEAGDQRRIMTPEQALSAGVDYMVIGRPVTQSVDPAQTLKAINASLQRSA

SEQ ID NO: 51 - protein, 295 aa, synthetic construct
MPQSTNAQPVGFGERLVAAGQVRGRLCVGIDPHPYLLQQWGLEDTLDGLRRFSEACVEAFADTAALVKPQVAFYERFGSQGFAVLEDTLQYLREAGCLTVADAKRGDIGSTMTGYADAWLGESSPLRADAVTVSPYLGVAALQPVFDLAAKTGRGVFVLAATSNPEAVQLQNFRDDSHTVAQHVVDTCAAFNSECAASSAQDEGANQHPVGDIGVVVGATLDTPPDLSQLNGPVLLPGVGAQGATAADVAKITQTAPQLGFPNVSRAILNAGPQVADLQKAVQETSREYFDGGLV

SEQ ID NO: 52 - protein, 241 aa, synthetic construct
MATNAKPVYKRILLKLSGEALQGTEGFGIDASILDRMAQEIKELVELGIQVGVVIGGGNLFRGAGLAKAGMNRVVGDHMGMLATVMNGLAMRDALHRAYVNARLMSAIPLNGVCDSYSWAEAISLLRNNRVVILSAGTGNPFFTTDSAACLRGIEIEADVVLKATKVDGVFTADPAKDPTATMYEQLTYSEVLEKELKVMDLAAFTLARDHKLPIRVFNMNKPGALRRVVMGEKEGTLITE

SEQ ID NO: 53 - protein, 241 aa, synthetic construct
MTTESRRTAYKRVMLKLGGEMFGGGQVGIDPDVVENVARQIAEIARSGTEVAVVIGGGNFFRGAELSQRGMDRARSDYMGMLGTVMNCLALQDFLGQLDVDCRVQTSINMAQVAEPYLPLRAVRHLEKGRVVIFGAGMGMPYFSTDTTAAQRALEIGCEVLFLAKGVDGVYSDDPRSNPDAELYDEITPREVIERGLKVADATAFSLCMDNYMPILVFNLLTEGNIARAVNGERIGTLVQS

SEQ ID NO: 54 - protein, 143 aa, synthetic construct
MAIERTFSIIKPNAVAKNVIGNIFARFEAAGFKIVGTKMLHLTVEQARGFYAEHDGKPFFDGLVEFMTSGPIVVSVLEGENAVQRHRDLLGATNPANALAGTLRADYADSLTENGTHGSDSVESAAREIAYFFGEGEVCPRTR

SEQ ID NO: 55 - protein, 136 aa, synthetic construct
MTERTLILIKPDGVTNGHVGEIIARIERKGLKLAALDLRVADRETAEKHYEEHADKPFFGELVEFITSAPLIAGIVEGERAIDAWRQLAGGTDPVAKATPGTIRGDFALTVGENVVHGSDSPESAEREISIWFPNL

SEQ ID NO: 56 - protein, 214 aa, synthetic construct
MRIILLGAPGAGKGTQAQFIMEKYGIPQISTGDMLRAAVKSGSELGKQAKDIMDAGKLVTDELVIALVKERIAQEDCRNGFLLDGFPRTIPQADAMKEAGINVDYVLEFDVPDELIVDRIVGRRVHAPSGRVYHVKFNPPKVEGKDDVTGEELTTRKDDQEETVRKRLVEYHQMTAPLIGYYSKEAEAGNTKYAKVDGTKPVAEVRADLEKILG

SEQ ID NO: 57 - protein, 181 aa, synthetic construct
MRLVLLGPPGAGKGTQAAILSEKLGIPHISTGDLFRANIGEGTPLGIEAKQYIDAGKLVPTDVTARMVASRLAESDAAEGFLLDGFPRTVEQADILANLLSEAGQTLDGVVNYQVSEDVVVERMLSRGRADDNEETIRTRLGVYRDETAPLIDHYGDKIINIEAEGEVEEINARTLKALGK

SEQ ID NO: 58 - protein, 491 aa, synthetic construct
MAVTQTAQACDLVIFGAKGDLARRKLLPSLYQLEKAGQLNPDTRIIGVGRADWDKAAYTKVVREALETFMKETIDEGLWDTLSARLDFCNLDVNDTAAFSRLGAMLDQKNRITINYFAMPPSTFGAICKGLGEAKLNAKPARVVMEKPLGTSLATSQEINDQVGEYFEECQVYRIDHYLGKETVLNLLALRFANSLFVNNWDNRTIDHVEITVAEEVGIEGRWGYFDKAGQMRDMIQNHLLQILCMIAMSPPSDLSADSIRDEKVKVLKSLRRIDRSNVREKTVRGQYTAGFAQGKKVPGYLEEEGANKSSNTETFVAIRVDIDNWRWAGVPFYLRTGKRLPTKCSEVVVYFKTPELNLFKESWQDLPQNKLTIRLQPDEGVDIQVLNKVPGLDHKHNLQITKLDLSYSETFNQTHLADAYERLLLETMRGIQALFVRRDEVEEAWKWVDSITEAWAMDNDAPKPYQAGTWGPVASVAMITRDGRSWNEFE

SEQ ID NO: 59 - protein, 468 aa, synthetic construct
MSKQQIGVVGMAVMGRNLALNIESRGYTVSIFNRSREKTEEVIAENPGKKLVPYYTVKEFVESLETPRRILLMVKAGAGTDAAIDSLKPYLDKGDIIIDGGNTFFQDTIRRNRELSAEGFNFIGTGVSGGEEGALKGPSIMPGGQKEAYELVAPILTKIAAVAEDGEPCVTYIGADGAGHYVKMVHNGIEYGDMQLIAEAYSLLKGGLNLTNEELAQTFTEWNNGELSSYLIDITKDIFTKKDEDGNYLVDVILDEAANKGTGKWTSQSALDLGEPLSLITESVFARYISSLKDQRVAASKVLSGPQAQPAGDKAEFIEKVRRALYLGKIVSYAQGFSQLRAASEEYNWDLNYGEIAKIFRAGCIIRAQFLQKITDAYAENPQIANLLLAPYFKQIADDYQQALRDVVAYAVQNGIPVPTFSAAVAYYDSYRAAVLPANLIQAQRDYFGAHTYKRIDKEGVFHTEWLD

SEQ ID NO: 60 - protein, 315 aa, synthetic construct
MPDMKLFAGNATPELAQRIANRLYTSLGDAAVGRFSDGEVSVQINENVRGGDIFIIQSTCAPTNDNLMELVVMVDALRRASAGRITAVIPYFGYARQDRRVRSARVPITAKVVADFLSSVGVDRVLTVDLHAEQIQGFFDVPVDNVFGSPILLEDMLQLNLDNPIVVSPDIGGVVRARAIAKLLNDTDMAIIDKRRPRANVSQVMHIIGDVAGRDCVLVDDMIDTGGTLCKAAEALKERGAKRVFAYATHPIFSGNAANNLRNSVIDEVVVCDTIPLSDEIKSLPNVRTLTLSGMLAEAIRRISNEESISAMFEH

SEQ ID NO: 61 - protein, 415 aa, synthetic construct
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 62 - protein, 793 aa, synthetic construct
MIEALRQQLLDDPRSWYAFLRHLVASQRDSWLYTDLQRACADFREQLPEGYAEGIGPLEDFVAHTQEVIFRDPWMVFAWRPRPGRWIYVRIHREQLALEELSTDAYLQAKEGIVGLGAEGEAVLTVDFRDFRPVSRRLRDESTIGDGLTHLNRRLAGRIFSDLAAGRSQILEFLSLHRLDGQNLMLSNGNTDFDSLRQTVQYLGTLPRETPWAEIREDMRRRGFAPGWGNTAGRVRETMRLLMDLLDSPSPAALESFLDRIPMISRILIVSIHGWFAQDKVLGRPDTGGQVVYILDQARALEREMRNRLRQQGVDVEPRILIATRLIPESDGTTCDQRLEPVVGAENVQILRVPFRYPDGRIHPHWISRFKIWPWLERYAQDLEREVLAELGSRPDLIIGNYSDGNLVATLLSERLGVTQCNIAHALEKSKYLYSDLHWRDHEQDHHFACQFTADLIAMNAADIIVTSTYQEIAGNDREIGQYEGHQDYTLPGLYRVENGIDVFDSKFNIVSPGADPRFYFSYARTEERPSFLEPEIESLLFGREPGADRRGVLEDRQKPLLLSMARMDRIKNLSGLAELYGRSSRLRGLANLVIIGGHVDVGNSRDAEEREEIRRMHEIMDHYQLDGQLRWVGALLDKTVAGELYRVVADGRGVFVQPALFEAFGLTVIEAMSSGLPVFATRFGGPLEIIEDGVSGFHIDPNDHEATAERLADFLEAARERPKYWLEISDAALARVAERYTWERYAERLMTIARIFGFWRFVLDRESQVMERYLQMFRHLQWRPLAHAVPME

SEQ ID NO: 63 - protein, 504 aa, synthetic construct [sequence omitted]

SEQ ID NO: 64 - protein, 339 aa, synthetic construct
MVIFVTGGAGYIGSHTILELLNNGHDVVSIDNFVNSSIESLKRVEQITNKKIISYQGDIRDKNLLDEIFSRHHIDAVIHFASLKSVGESKLKPLEYYSNNVGGTLVLLECMKRYNINKMIFSSSATVYGSNSIPPHTEDRRIGETTNPYGTSKFIIEIILSDYCDSDNNKSVIALRYFNPIGAHKSGMIGENPNGIPNNLVPYISKVAQNQLPVLNIYGNDYPTKDGTGVRDYIHVCDLAKGHVKALEYMFLNDVNYEAFNLGTGQGYSVLEIVKMFEIVTKKSIPVAICNRREGDVAESWASADLAHKKLSWKAEKNFERMIEDVWRWQTNNPNGYKK

SEQ ID NO: 65 - protein, 388 aa, synthetic construct
MKITISGTGYVGLSNGLLIAQNHEVVALDILPSRVAMLNDRISPIVDKEIQQFLQSDKIHFNATLDKNEAYRDADYVIIATPTDYDPKTNYFNTSSVESVIKDVVEINPYAVMVIKSTVPVGFTAAMHKKYRTENIIFSPEFLREGKALYDNLHPSRIVIGERSERAERFAALLQEGAIKQNIPMLFTDSTEAEAIKLFANTYLAMRVAYFNELDSYAESLGLNSRQIIEGVCLDPRIGNHYNNPSFGYGGYCLPKDTKQLLANYQSVPNNLISAIVDANRTRKDFIADAILSRKPQVVGIYRLIMKSGSDNFRASSIQGIMKRIKAKGVEVIIYEPVMKEDSFFNSRLERDLATFKQQADVIISNRMAEELKDVADKVYTRDLFGSD

SEQ ID NO: 66 - protein, 815 aa, synthetic construct
MNAPFTYSSPTLSVEALKHSIAYKLMFTIGKDPVVANKHEWLNATLFAVRDRLVERWLRSNRAQLSQETRQVYYLSMEFLIGRTLSNAMLSLGIYEDVQGALEAMGLNLEELIDEENDPGLGNGGLGRLAACFLDSLATLGLPGRGYGIRYDYGMFKQNIVNGSQKESPDYWLEYGNPWEFKRHNTRYKVRFGGRIQQEGKKTRWIETEEILGVAYDQIIPGYDTDATNTLRLWSAQASSEINLGKFNQGDYFAAVEDKNHSENVSRVLYPDDSTYSGRELRLRQEYFLVSSTIQDILSRHYQLHKTYDNLADKIAIHLNDTHPVLSIPEMMRLLIDEHQFSWDDAFEVCCQVFSYTNHTLMSEALETWPVDMLGKILPRHLQIIFEINDYFLKTLQEQYPNDTDLLGRASIIDESNGRRVRMAWLAVVVSHKVNGVSELHSNLMVQSLFADFAKIFPGRFTNVTNGVTPRRWLAVANPSLSAVLDEHLGRNWRTDLSLLNELQQHCDFPMVNHAVHQAKLENKKRLAEYIAQQLNVVVNPKALFDVQIKRIHEYKRQLMNVLHVITRYNRIKADPDAKWVPRVNIFGGKAASAYYMAKHIIHLINDVAKVINNDPQIGDKLKVVFIPNYSVSLAQLIIPAADLSEQISLAGTEASGTSNMKFALNGALTIGTLDGANVEMLDHVGADNIFIFGNTAEEVEELRRQGYKPREYYEKDEELHQVLTQIGSGVFSPEDPGRYRDLVDSLINFGDHYQVLADYRSYVDCQDKVDELYELQEEWTAKAMLNIANMGYFSSDRTIKEYADHIWHIDPVRL

SEQ ID NO: 67 - protein, 511 aa, synthetic construct
MMKMRWLSAAVMLTLYTSSSWAFSIDDVAKQAQSLAGKGYETPKSNLPSVFRDMKYADYQQIQFNHDKAYWNNLKTPFKLEFYHQGMYFDTPVKINEVTATAVKRIKYSPDYFTFGDVQHDKDTVKDLGFAGFKVLYPINSKDKNDEIVSMLGASYFRVIGAGQVYGLSARGLAIDTALPSGEEFPRFKEFWIERPKPTDKRLTIYALLDSPRATGAYKFVVMPGRDTVVDVQSKIYLRDKVGKLGVAPLTSMFLFGPNQPSPANNYRPELHDSNGLSIHAGNGEWIWRPLNNPKHLAVSSFSMENPQGFGLLQRGRDFSRFEDLDDRYDLRPSAWVTPKGEWGKGSVELVEIPTNDETNDNIVAYWTPDQLPEPGKEMNFKYTITFSRDEDKLHAPDNAWVQQTRRSTGDVKQSNLIRQPDGTIAFVVDFTGAEMKKLPEDTPVTAQTSIGDNGEIVESTVRYNPVTKGWRLVMRVKVKDAKKTTEMRAALVNADQTLSETWSYQLPANE

SEQ ID NO: 68 - protein, 549 aa, synthetic construct
MKNINPTQTAAWQALQKHFDEMKDVTIADLFAKDGDRFSKFSATFDDQMLVDYSKNRITEETLAKLQDLAKECDLAGAIKSMFSGEKINRTENRAVLHVALRNRSNTPILVDGKDVMPEVNAVLEKMKTFSEAIISGEWKGYTGKAITDVVNIGIGGSDLGPYMVTEALRPYKNHLNMHFVSNVDGTHIAEVLKKVNPETTLFLVASKTFTTQETMTNAHSARDWFLKAAGDEKHVAKHFAALSTNAKAVGEFGIDTANMFEFWDWVGGRYSLWSAIGLSIVLSIGFDNFVELLSGAHAMDKHFSTTPAEKNLPVLLALIGIWYNNFFGAETEAILPYDQYMHRFAAYFQQGNMESNGKYVDRNGNVVDYQTGPIIWGEPGTNGQHAFYQLIHQGTKMVPCDFIAPAITHNPLSDHHQKLLSNFFAQTEALAFGKSREVVEQEYRDQGKDPATLDYVVPFKVFEGNRPTNSILLREITPFSLGALIALYEHKIFTQGVILNIFTFDQWGVELGKQLANRILPELKDDKEISSHDSSTNGLINRYKAWRG

SEQ ID NO: 69 - protein, 550 aa, synthetic construct
MKLLQRGVALALLTTFTLASETALAYEQDKTYKITVLHTNDHHGHFWRNEYGEYGLAAQKTLVDGIRKEVAAEGGSVLLLSGGDINTGVPESDLQDAEPDFRGMNLVGYDAMAIGNHEFDNPLTVLRQQEKWAKFPLLSANIYQKSTGERLFKPWALFKRQDLKIAVIGLTTDDTAKIGNPEYFTDIEFRKPADEAKLVIQELQQTEKPDIIIAATHMGHYDNGEHGSNAPGDVEMARALPAGSLAMIVGGHSQDPVCMAAENKKQVDYVPGTPCKPDQQNGIWIVQAHEWGKYVGRADFEFRNGEMKMVNYQLIPVNLKKKVTWEDGKSERVLYTPEIAENQQMISLLSPFQNKGKAQLEVKIGETNGRLEGDRDKVRFVQTNMGRLILAAQMDRTGADFAVMSGGGIRDSIEAGDISYKNVLKVQPFGNVVVYADMTGKEVIDYLTAVAQMKPDSGAYPQFANVSFVAKDGKLNDLKIKGEPVDPAKTYRMATLNFNATGGDGYPRLDNKPGYVNTGFIDAEVLKAYIQKSSPLDVSVYEPKGEVSWQ

SEQ ID NO: 70 - protein, 382 aa, synthetic construct
MSLKEKTQSLFANAFGYPATHTIQAPGRVNLIGEHTDYNDGFVLPCAIDYQTVISCAPRDDRKVRVMAADYENQLDEFSLDAPIVAHENYQWANYVRGVVKHLQLRNNSFGGVDMVISGNVPQGAGLSSSASLEVAVGTVLQQLYHLPLDGAQIALNGQEAENQFVGCNCGIMDQLISALGKKDHALLIDCRSLGTKAVSMPKGVAVVIINSNFKRTLVGSEYNTRREQCETGARFFQQPALRDVTIEEFNAVAHELDPIVAKRVRHILTENARTVEAASALEQGDLKRMGELMAESHASMRDDFEITVPQIDTLVEIVKAVIGDKGGVRMTGGGFGGCIVALIPEELVPAVQQAVAEQYEAKTGIKETFYVCKPSQGAGQC

SEQ ID NO: 71 - protein, 348 aa, synthetic construct
MTQFNPVDHPHRRYNPLTGQWILVSPHRAKRPWQGAQETPAKQVLPAHDPDCFLCAGNVRVTGDKNPDYTGTYVFTNDFAALMSDTPDAPESHDPLMRCQSARGTSRVICFSPDHSKTLPELSVAALTEIVKTWQEQTAELGKTYPWVQVFENKGAAMGCSNPHPHGQIWANSFLPNEAEREDRLQKEYFAEQKSPMLVDYVQRELADGSRTVVETEHWLAVVPYWAAWPFETLLLPKAHVLRITDLTDAQRSDLALALKKLTSRYDNLFQCSFPYSMGWHGAPFNGEENQHWQLHAHFYPPLLRSATVRKFMVGYEMLAETQRDLTAEQAAERLRAVSDIHFRESGV

SEQ ID NO: 72 - protein, 300 aa, synthetic construct
MTGAFDNHSFRTAIVPAAGLGTRFLPTTKAVPKELLPVVDTPGIEIVASEAAQAGAKRLVIVTSPGKESVVSYFRPAPELEANLERKGKTALLEKVRRGPELIEVEVAIQEEALGLGHAVAQAEPNLTDEDEAVAVLLPDDLVLPAGVLSKMAEVRAEHGGSVLCAFDIPKEQISPYGVFDVTDTGDPDVKQVHGMVEKPSPEEAPSTYAAAGRYLLDRAIFDALRRIEPGAGGELQLTDAVALLIEEGHPVHIVVHRGGRHDLGNPGGFLKAAVDFALDDPDYGPELRNWLTERLGTDR

SEQ ID NO: 73 - protein, 299 aa, synthetic construct
MTASAEHQTFTTAIVPAAGLGTRFLPTTKSVPKELLPVVDTPAIELVADEARQAGAERLVIVTSPAKQSIAAYFRPAPELERSLEEKGKTGQLAKIRRAPELLEVEVAIQEQALGLGHAVACAEPNLGPEDDVVAVLLPDDLVLPHGILERMAKVRAEHGGSVLCAFDIPKEEISAYGVFDVSDTDDADVKRVHGMVEKPPAEQAPSTFAAAGRYLLDRAIFDALRRIEPGAGGELQLTDAVALLIQEGHPVHVVVHRGDRHDLGNPGGFLRAAVDFALQDPDYGPELRAWLTDRIARP

SEQ ID NO: 74 - protein, 296 aa, synthetic construct
MSPTSAFRTAIVPAAGLGTRFLPTTKAVPKELLPVVDTPGIEMVAAEAADAGASRLVIVTSPGKESVAEYFQPRPELEATLEQRGKTELLAKVRRATGLIEVETALQEQALGLGHAVGCAEPNLNGSDDAVAVLLPDDLVLPAGVLIKMSEVRAQRGGSVLCAFDIPKEQISAYGVFDVADTEDPDVKRVNGMVEKPPADQAPSTFAAAGRYLLDRAVFDALKRIEPGAGGELQLTDAIALLIAEGHPVHVVVHRGGRHDLGNPGGFLRAAVDFALDHPEYGPDLRSWLRNRVGGE

SEQ ID NO: 75 - protein, 304 aa, synthetic construct
MSAKVWVLGDAVVDLLPESDGRLLPCPGGAPANVAVGIARLGGTSGFIGRVGDDPFGALMQRTLLTEGVDITYLKQDEWHRTSTVLVDLNDQGERSFTFMVRPSADLFLETTDLPCWRHGEWLHLCSIALSAEPSRTSAFTAMTAIRHAGGFVSFDPNIREDLWQDEHLLRLCLRQALQLADVVKLSEEEWRLISGKTQNDQDICALAKEYEIAMLLVTKGAEGVVVCYRGQVHHFAGMSVNCVDSTGAGDAFVAGLLTGLSSTGLSTDEREMRRIIDLAQRCGALAVTAKGAMTALPCRQELE

SEQ ID NO: 76 - protein, 374 aa, synthetic construct
MIVAFCLYKYFPFGGLQRDFMRIASTVAARGHHVRVYTQSWEGDCPKAFELIQVPVKSHTNHGRNAEYYAWVQNHLKEHPADRVVGFNKMPGLDVYFAADVCYAEKVAQEKGFLYRLTSRYRHYAAFERATFEQGKSTKLMMLTDKQIADFQKHYQTEPERFQILPPGIYPDRKYSEQIPNSREIYRQKNGIKEQQNLLLQVGSDFGRKGVDRSIEALASLPESLRHNTLLFVVGQDKPRKFEALAEKLGVRSNVHFFSGRNDVSELMAAADLLLHPAYQEAAGIVLLEAITAGLPVLTTAVCGYAHYIADANCGTVIAEPFSQEQLNEVLRKALTQSPLRMAWAENARHYADTQDLYSLPEKAADIITGGLDG

SEQ ID NO: 77 - protein, 474 aa, synthetic construct
MSRLVVVSNRIAPPDEHAASAGGLAVGILGALKAAGGLWFGWSGETGNEDQPLKKVKKGNITWASFNLSEQDLDEYYNQFSNAVLWPAFHYRLDLVQFQRPAWDGYLRVNALLADKLLPLLQDDDIIWIHDYHLLPFAHELRKRGVNNRIGFFLHIPFPTPEIFNALPTYDTLLEQLCDYDLLGFQTENDRLAFLDCLSNLTRVTTRSAKSHTAWGKAFRTEVYPIGIEPKEIAKQAAGPLPPKLAQLKAELKNVQNIFSVERLDYSKGLPERFLAYEALLEKYPQHHGKIRYTQIAPTSRGDVQAYQDIRHQLENEAGRINGKYGQLGWTPLYYLNQHFDRKLLMKIFRYSDVGLVTPLRDGMNLVAKEYVAAQDPANPGVLVLSQFAGAANELTSALIVNPYDRDEVAAALDRALTMSLAERISRHAEMLDVIVKNDINHWQECFISDLKQIVPRSAESQQRDKVATFPKLA

SEQ ID NO: 78 - protein, 413 aa, synthetic construct
MNKTLIAAAVAGIVLLASNAQAQTVPEGYQLQQVLMMSRHNLRAPLANNGSVLEQSTPNKWPEWDVPGGQLTTKGGVLEVYMGHYMREWLAEQGMVKSGECPPPYTVYAYANSLQRTVATAQFFITGAFPGCDIPVHHQEKMGTMDPTFNPVITDDSAAFSEQAVAAMEKELSKLQLTDSYQLLEKIVNYKDSPACKEKQQCSLVDGKNTFSAKYQQEPGVSGPLKVGNSLVDAFTLQYYEGFPMDQVAWGEIKSDQQWKVLSKLKNGYQDSLFTSPEVARNVAKPLVSYIDKALVTDRTSAPKITVLVGHDSNIASLLTALDFKPYQLHDQNERTPIGGKIVFQRWHDSKANRDLMKIEYVYQSAEQLRNADALTLQAPAQRVTLELSGCPIDADGFCPMDKFDSVLNEAVK